(19) United States
(12) Patent Application Publication
(10) Pub. No.: US 2025/0143173 A1
(43) Pub. Date: May 1, 2025

(54) PEROVSKITE LAYER, FABRICATION METHOD AND USE THE SAME

(71) Applicant: City University of Hong Kong, Kowloon (HK)

(72) Inventors: Alex Kwan Yue Jen, Kowloon (HK); Shengfan Wu, Kowloon (HK); Yichao Yan, Kowloon (HK)

(21) Appl. No.: 18/499,651

(22) Filed: Nov. 1, 2023

(51) Int. Cl. H10K 85/60; C07C 211/63; C07C 309/44; H10K 30/40; H10K 30/57; H10K 85/20; H10K 85/50

(52) U.S. Cl. CPC H10K 85/615; C07C 211/63; C07C 309/44; H10K 30/40; H10K 30/57; H10K 85/215; H10K 85/50; C07C 2603/24

(57) ABSTRACT

A perovskite layer for use in a solar cell includes a mixture of a halide perovskite and a sulfonyl naphthoquinone-based compound having a structure of Formula (I). Methods for fabricating the perovskite layer and a solar cell including a first active layer of the perovskite layer are also addressed.

### TECHNICAL FIELD

The present invention relates to a perovskite layer for example particularly, but not exclusively, a perovskite layer comprising a mixture of a halide perovskite and a sulfonyl naphthoquinone-based compound; and a method for fabricating the perovskite layer. Also pertaining to the present invention is a solar cell comprising a first active layer of the perovskite layer.

### BACKGROUND OF THE INVENTION

Metal halide perovskite solar cells (PSCs) are promising photovoltaic (PV) technologies owing to their high absorption coefficient, low exciton binding energy, high carrier mobility, long carrier diffusion length and ambipolar charge transport. PSCs may generally be configured as single-junction solar cells and multi-junction (or tandem) solar cells (i.e., solar cells consisting of two or more sub-cells). In particular, since tandem configurations would allow combination of sub-cells with different properties (such as the combination of a wide-bandgap and a low-bandgap subcells that could cater both high-energy and low-energy photons upon operation), it is believed that PSCs with tandem configurations would have a higher power conversion efficiency (PCE) as compared with those having single-junction configurations. For example, in perovskite-organic tandem solar cells (PO-TSCs) with a front sub-cell having a desired bandgap (E_{g}) of about 1.8 to 1.9 eV, its PCE may be increased from about 15% to about 24%, particularly when employing I/Br mixed perovskites (FIG. 1A).

However, the perovskite layer of PSCs usually suffers from severe halide phase segregation during device operation, which is believed to be a result of halide migration through halide vacancies within the perovskite film, posing a significant obstacle to the long-term stability of perovskite-based PSCs. In addition, under continuous illumination and/or heating, Pb²⁺ ions in perovskites are also prone to be reduced to metallic Pb⁰, which is detrimental to device efficiency and stability.

Whilst there are reports on suppressing halide segregation by, for example, crystallization control, defect passivation, surface modulation, and strain engineering, it is believed that none of these strategies could address the facile reduction of Pb²⁺ to Pb⁰ at the same time. In addition, the additives used in the reported strategies are either inorganic compounds, which are believed to be hard to tune their properties synthetically; or sacrificial agents that are believed to diminish soon after taking effects. That said, it remains a challenge to have an additive that is facile to tailor its properties for elimination different defects and can function in a sustainable manner without introducing additional deep-level defects.

The invention seeks to eliminate or at least to mitigate such shortcomings by providing a new or otherwise improved solar cell active layer, particularly an active perovskite layer that is capable of enabling selective iodine (I₂) reduction and metallic lead (Pb⁰) oxidation in a sustainable manner.

### SUMMARY OF THE INVENTION

In a first aspect of the present invention, there is provided a perovskite layer for use in a solar cell comprising a mixture of a halide perovskite and a sulfonyl naphthoquinone-based compound having a structure of Formula (I):

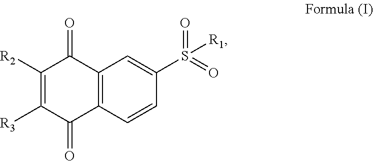

wherein:
R₁ is independently selected from one of OM, OR₄, and NR₅R₆, with M being a cation, R₄, R₅ and R₆ being independently selected from a hydrogen and a substituent; and R₂ and R₃ are independently selected from a hydrogen and a substituent, or R₂ and R₃ may form a fused ring.

Optionally, M is selected from the group consisting of H⁺, Na⁺, K⁺, Cs⁺, Rb⁺, Sr²⁺, Ca²⁺, Ni²⁺, Cu²⁺, Co²⁺, Zn²⁺, Mg²⁺, Ba²⁺, Li⁺, ammonium ion, and aminum ion; R₂, R₃, R₄, R₅ and R₆ are independently selected from the group consisting of hydrogen, substituted or unsubstituted C1 to C4 alkyl, substituted or unsubstituted C2 to C6 alkenyl, substituted or unsubstituted C2 to C6 alkynyl, halogen, substituted or unsubstituted C6 to C10 aryl, —CN, —C(═O)OH, —C(═O)H, —C(═O)R₇, —OR₇, —SH, —SR₇, —NH₂, —NHR₆, —N(R₇)₂, —Si(R₇)₃, —OSi(R₇)₃, —S(O)OH and —P(O)(OH)₂ where R₇ is an alkyl or a phenyl; and when R₂ and R₃ form a fused ring, it comprises a substituted or unsubstituted 6-membered to 14-membered ring.

In an optional embodiment, the sulfonyl naphthoquinone-based compound is any one of Formula (II), (III), (IV), (V), (VI), or (VII):

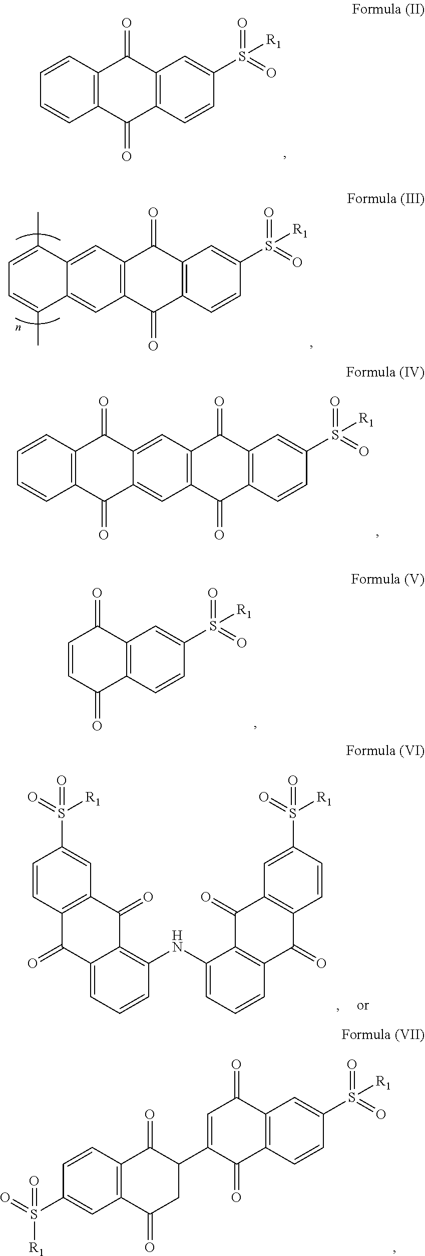

with R₁ as defined above; and n being 1-1000.

It is optional that the sulfonyl naphthoquinone-based compound of Formula (II) is selected form any one of Formula (VIII), Formula (IX), or Formula (X):

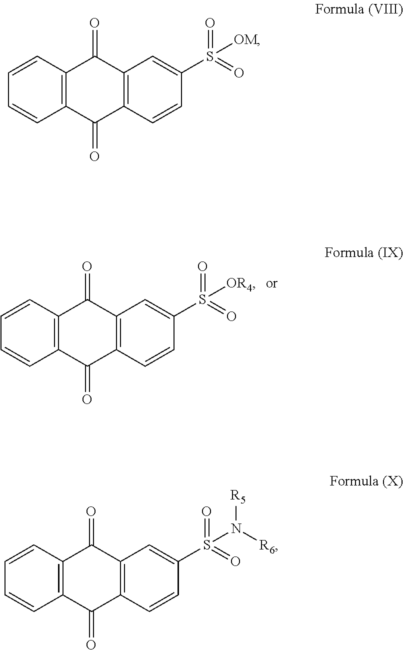

with M being H⁺, ammonium ion or aminum ion; R₄ being C1 to C4 alkyl; R₅ and R₆ being independently selected from hydrogen and C1 to C4 alkyl.

In an embodiment of the invention, the sulfonyl naphthoquinone-based compound of Formula (VIII) is selected from any one of the Formula (VIIIa), (VIIIb), and (VIIIc):

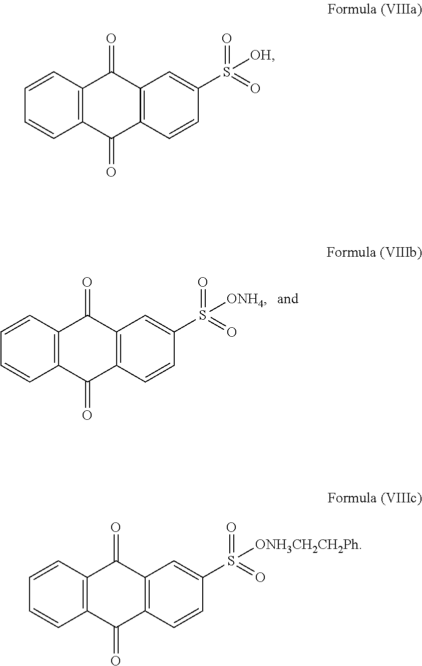

In an optional embodiment, the sulfonyl naphthoquinone-based compound comprises a structure of Formula (IXa):

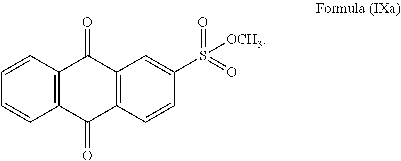 [SMILES: COS(=O)(=O)c1ccc2c(c1)C(=O)c1ccccc1C2=O]

In an optional embodiment, the sulfonyl naphthoquinone-based compound comprises a structure of Formula (Xa):

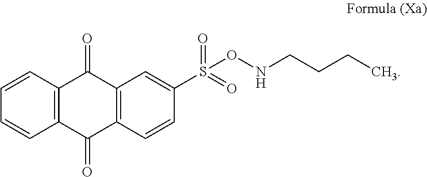 [SMILES: CCCCNOS(=O)(=O)c1ccc2c(c1)C(=O)c1ccccc1C2=O]

Optionally, the halide perovskite comprises a crystal grain structure of [A⁺¹B⁺²X⁻¹₃], with A⁺¹ being an A-site monovalent cation, B⁺² being a B-site divalent cation, and X⁻¹ being a halide anion.

It is optional that the A-site monovalent cation is selected from the group consisting of formamidinium (FA⁺), methylammonium (MA⁺), ethylammonium (EA⁺), guanidinium (GA⁺), Cs⁺, Rb⁺ and a combination thereof; the B-site divalent cation is selected from the group consisting of Pb²⁺, Sn²⁺, Ge²⁺ and a combination thereof; and the halide anion is selected from the group consisting of I⁻, Br⁻, Cl⁻ and a combination thereof.

In an optional embodiment, the halide perovskite is selected from any one of CsPb_{0.5}Sn_{0.5}I₃, FAPbI₃, MA_{0.25}FA_{0.75}PbI_{2.2}Br_{0.6}Cl_{0.2}, Cs_{0.02}FA_{0.96}MA_{0.02}PbI_{0.99}Cl_{0.01}, MAPb_{0.92}Sn_{0.08}I₃, (FA_{0.95}MA_{0.05})_{0.95}Cs_{0.05}Pb(I_{0.96}Br_{0.04})₃, Rb_{0.1}FA_{0.8}GA_{0.1}Pb_{0.6}Ge_{0.4}I₃, and (FA_{0.92}MA_{0.05})_{0.9}Cs_{0.1}Pb(I_{0.92}Br_{0.08})₃, and Cs_{0.2}FA_{0.8}Pb(I_{0.6}Br_{0.4})₃.

Optionally, the crystal grain structure of [A⁺¹B⁺²X⁻¹₃] includes grain boundaries at which the sulfonyl naphthoquinone-based compound accommodates.

It is optional that the perovskite layer contains about 0.3 mol % to about 1 mol % of the sulfonyl naphthoquinone-based compound.

In an optional embodiment, the perovskite layer further comprises about 3 mol % of MAPbCl₃ and 5 mol % of 4-guanidinobenzoic acid.

Optionally, the perovskite layer has a thickness of about 260 nm.

In a second aspect of the present invention, there is provided a method for fabricating the perovskite layer in accordance with the first aspect, comprising the steps of:
a) providing a solution mixture including a halide perovskite precursor and the sulfonyl naphthoquinone-based compound in accordance with the first aspect; b) spin-coating the solution mixture on a substrate; and c) annealing the spin-coated solution mixture to form the perovskite layer.

Optionally, step a) comprises the steps of: a1) providing a first reaction mixture including halides of formamidinium, methylammonium, and cesium; and a2) mixing the first reaction mixture with about 0.3 mol % to about 1 mol % of the sulfonyl naphthoquinone-based compound to form a second reaction mixture.

In an optional embodiment, the halides comprises CsI, CsBr, FAI, FABr, PbI₂, and PbBr₂.

It is optional that the first reaction mixture further comprises about 3 mol % of MAPbCl₃ and about 5 mol % of 4-guanidinobenzoic acid.

Optionally, step a) further comprises step a3) converting sodium anthraquinone-2-sulfonate to the sulfonyl naphthoquinone-based compound.

It is optional that step b) comprises step b1) adding an anti-solvent agent of chlorobenzene onto the center of the spin-coated solution mixture about 10 seconds before completing the spin-coating process.

Optionally, the spin-coating is performed at about 4000 rpm to about 6500 rpm with a ramping rate of about 1500 rpm si.

In an optional embodiment, the method, after step c), further comprises the steps of: spin-coating a surface passivating agent of piperazinium iodide (PI) on the perovskite layer; and annealing the PI-coated perovskite layer.

In a third aspect of the present invention, there is provided a solar cell including: a first hole transport layer; a first electron transport layer; and a first active layer of the perovskite layer in accordance with the first aspect that is disposed between the first hole transport layer and the first electron transport layer.

Optionally, the first active layer is in direct contact with the first hole transport layer and the first electron transport layer.

In an optional embodiment, the first hole transport layer is disposed on a transparent conductive layer that is disposed on a transparent substrate and the first electron transport layer is disposed on a first blocking layer that is disposed on a first metal layer.

In an embodiment of the invention, the first hole transport layer is in direct contact with a transparent conductive layer and the first electron transport layer is in direct contact with the first blocking layer.

It is optional that the transparent substrate is selected from the group consisting of glass, polymethyl methacrylate (PMMA), polycarbonate (PC), general-purpose polystyrene (GPPS), polyethylene glycol terephthalate (PET), polyethylene naphthalate (PEN), polydimethylsiloxane (PDMS), styrene-ethylene-butylene-styrene (SEBS), ethylene terephthalateco-1,4-cylclohexylenedimethylene terephthalate (PETG), acrylonitrile butadiene styrene copolymers (ABS), polypropylene (PP), polyamide (PA), acrylonitrile-styrene copolymer (AS), and a combination thereof.

Optionally, the transparent conductive layer is selected from the group consisting of Indium Tin Oxide (ITO), Aluminum Zinc Oxide (AZO), Fluorine Tin Oxide (FTO), graphene, poly(3,4-ethylenedioxythiophene):poly(styrene sulfonate) (PEDOT:PSS), Ag nanowire, Cu nanowire and a combination thereof.

It is optional that the first hole transport layer is selected from the group consisting of poly(triaryl amine) (PTAA), PEDOT:PSS, NiOx, 2,2′,7,7′-Tetrakis[N,N-di(4-methoxyphenyl)amino]-9,9′-spirobifluorene (Spiro-OMeTAD), DC-PA, MoOₓ and a combination thereof.

Optionally, the first electron transport layer is selected from the group consisting of PC₆₁BM, C₆₀, SnO₂, PNDIT-F3N and a combination thereof.

It is optional that the first blocking layer is selected from the group consisting of bathocuproine (BCP), bis-C₆₀, SnOₓ, Zr(acac)₂, MoOₓ and a combination thereof.

Optionally, the first metal layer is selected from the group consisting of Ag, Cu, Au, Al, W, Fe, Pt and a combination thereof.

In an optional embodiment, the solar cell comprises a band gap of about 1.81 eV.

Optionally, the solar cell comprises a power conversion efficiency of about 18.98% to about 19.58%.

It is optional that the solar cell comprises a power conversion efficiency of 95% of its initial value upon AM1.5G illumination for 500 hours at about 45° C.

In an optional embodiment, the solar cell further comprises a subcell disposed on and in direct contact with the first metal layer.

It is optional that the solar cell further includes an anti-reflection layer to which the transparent conductive layer and the transparent substrate are disposed on and in direct contact therewith.

Optionally, the anti-reflection layer is selected from the group consisting of MgF₂, LiF, PDMS and a combination thereof.

It is optional that the subcell comprises: a second hole transport layer; a second electron transport layer; and a second active layer that is disposed between the second hole transport layer and the second electron transport layer.

In an optional embodiment, the second active layer is in direct contact with the second hole transport layer and the second electron transport layer.

Optionally, the second active layer is selected from the group consisting of a perovskite photovoltaic material, a Si photovoltaic material, a CIGS photovoltaic material, a CdTe photovoltaic material, an organic photovoltaic material and a combination thereof.

It is optional that the second hole transport layer is disposed on the first metal layer and the second electron transport layer is disposed on a second metal layer.

Optionally, the second hole transport layer is in direct contact with the first metal layer and the second electron transport layer is in direct contact with the second metal layer.

In an optional embodiment, the second hole transport layer is disposed on the first metal layer and the second electron transport layer is disposed on the second metal layer.

Optionally, the second hole transport layer is in direct contact with the first metal layer and the second electron transport layer is in direct contact with the second blocking layer.

In an optional embodiment, the solar cell comprises a perovskite-organic tandem solar cell.

Optionally, the perovskite-organic tandem solar cell comprises the first active layer of Cs_{0.2}FA_{0.8}Pb(I_{0.6}Br_{0.4})₃ and the second active layer of PM6:Y6:P₇₁BM.

It is optional that the perovskite-organic tandem solar cell has a power conversion efficiency of about 24.27% to about 25.22%.

Optionally, the perovskite-organic tandem solar cell has a power conversion efficiency of 92% of its initial value upon AM1.5G illumination for 500 hours at about 45° C.

### BRIEF DESCRIPTION OF DRAWINGS

The invention will now be more particularly described, by way of example only, with reference to the accompanying drawings, in which:

FIG. 1A shows the theoretically achievable efficiency for two-junction solar cells with a 2-terminal configuration;

FIG. 1B is a schematic diagram illustrating the exemplary solar cell in accordance with an embodiment of the present invention;

FIG. 1C is a schematic diagram illustrating the exemplary solar cell in accordance with an embodiment of the present invention;

FIG. 2A is a ¹H NMR spectrum of the as-synthesized AQSH (Compound of Formula (VIIIa);

FIG. 2B is a ¹³C NMR spectrum of the as-synthesized AQSH (Compound of Formula (VIIIa);

FIG. 3A is a ¹H NMR spectrum of the as-synthesized AQSN (Compound of Formula (VIIIb);

FIG. 3B is a ¹³C NMR spectrum of the as-synthesized AQSN (Compound of Formula (VIIIb);

FIG. 4A is a ¹H NMR spectrum of the as-synthesized AQSP (Compound of Formula (VIIIc);

FIG. 4B is a ¹³C NMR spectrum of the as-synthesized AQSP (Compound of Formula (VIIIc);

FIG. 5 is a photograph showing I⁰ and Pb⁰ powder dispersed in DMF/IPA mixed solvent (volume ratio 1:10) with anthraquinone (after stirring at 100° C. for 60 min);

FIG. 6A is a schematic diagram illustrating the molecular structure of the organic redox mediators based on AQS core with different cationic substituents;

FIG. 6B shows the cyclic voltammetry curve of AQSH;

FIG. 7 is a schematic diagram illustrating the synthetic route of AQSH, AQSP, and AQSN;

FIG. 8 is a schematic diagram illustrating the electron shuttling between AQS core, Pb, and I;

FIG. 9 shows the transmission spectra of Pb⁰ and I⁰ powder dispersed in IPA:DMF mixed solvent (v:v=10:1) with and without AQSH;

FIG. 10A shows the transmission spectra of I₂, FAI, and FAI+AQSH powder dissolved in DMF;

FIG. 10B shows the photographs of the solution consisting of AQSH with I⁰ powder (left) and AQSH with Pb⁰ powder (right);

FIG. 11 shows the powder X-ray diffraction (XRD) patterns of PbI₂ and the bottom precipitates taken from the glass vial as shown in the inset of FIG. 9;

FIG. 12 is a schematic diagram illustrating the proposed mechanism of sustainable elimination of metallic Pb⁰ and I⁰ species in perovskite layer with the redox mediator as an additive, and its effect on suppressing halide segregation;

FIG. 13 shows the time-dependent photoluminescence (tdPL) spectra of I/Br mixed perovskite with 0.5 mol % of different AQS derivatives;

FIG. 14 shows the normalized PL spectra of the I/Br mixed perovskite films with different redox mediators after being illuminated for 0, 5, and 10 min (extracted from the corresponding time-dependent PL spectra in FIG. 13);

FIG. 15 shows the as-derived PL spectra for analyzing phase stability of I/Br mixed perovskite with 0.5 mol % of different AQS derivatives;

FIG. 16 shows the time-dependent PL spectra of the I/Br mixed perovskite films incorporated with NH₄I or PEAI as additive;

FIG. 17 shows the photograph of the aging condition for the mix-halide perovskite films with different AQS derivatives as additive, which were sealed in a 20 mL vial (filled with N₂) and illuminated for two days. The intensity of the simulated AM1.5G illumination is calibrated to 1-sun equivalent intensity;

FIG. 18A shows the high-resolution X-ray photoelectron spectroscopy (XPS) spectra of Pb 4f of the aged perovskite films with different AQS derivatives;

FIG. 18B shows the high-resolution X-ray photoelectron spectroscopy (XPS) spectra of I 3d of the aged perovskite films with different AQS derivatives;

FIG. 18C shows the high-resolution X-ray photoelectron spectroscopy (XPS) spectra of Br 3d of the aged perovskite films with different AQS derivatives;

FIG. 19 shows the atomic ratio of metallic Pb⁰ in total Pb element of the perovskite films with or without different AQS derivatives;

FIG. 20A shows the high-resolution XPS spectra of metallic Pb (Pb⁰) in total Pb (Pb²⁺+Pb⁰) element of the pristine perovskite films with different AQS derivatives;

FIG. 20B shows the atomic ratio of metallic Pb (Pb⁰) in total Pb (Pb²⁺+Pb⁰) element of the pristine perovskite films with or without different AQS derivatives;

FIG. 21 shows the atomic ratio of I:Pb, I:Br, and (I+Br):Pb in the perovskite films with or without different different AQS derivatives;

FIG. 22 shows the top-viewing SEM images of the perovskite films with or without AQS derivatives;

FIG. 23 shows the thin film XRD patterns of the perovskite films with or without AQS derivatives;

FIG. 24 shows the cross-sectional SEM image and EDX elemental distribution (line scan for S, Pb, and In elements) for the perovskite film (prepared on ITO substrate) with AQSH as the additive;

FIG. 25 shows the UV-vis absorption spectra of the perovskite films with or without AQS derivatives;

FIG. 26A shows the optimized slab structures for pristine FAI-rich and defective FAI-rich (100)/(110) upon density functional theory (DFT) calculations based on redox mediator/perovskite interfacial models;

FIG. 26B shows the optimized slab structures for pristine PbI₂-rich and defective PbI₂-rich (100)/(110) upon density functional theory (DFT) calculations based on redox mediator/perovskite interfacial models;

FIG. 27A is a schematic diagram illustrating the half-reactions between AQS core, I⁰, and Pb⁰;

FIG. 27B shows the calculated energy barrier for the half-reaction from I⁰ to I⁻ and Pb⁰ to Pb²⁺, and the total energy barrier for redox charge shuttling;

FIG. 28 shows the surface Pb²⁺ binding energy for the PbI₂-rich FAPbI₃ (100) and (110) surface with or without AQS adsorption;

FIG. 29 shows the calculated binding energies between NH₄⁺ (PEA⁺) and pristine/defective FAPbI₃ (100) and (110) surfaces;

FIG. 30A shows the optimized crystal structure for pristine PbI₂-rich (100) with AQS adsorption. The optimization were performed at GGA/PBE+vdW level of theory;

FIG. 30B shows the optimized crystal structure for pristine PbI₂-rich (110) with AQS adsorption. The optimization were performed at GGA/PBE+vdW level of theory;

FIG. 31A shows the optimized crystal structure for pristine FAI-rich (100) with NH₄⁺ and PEA⁺ adsorption. The optimizations were performed at GGA/PBE+vdW level of theory;

FIG. 31B shows the optimized crystal structure for defective FAI-rich (100)-V_{FA} with NH₄⁺ and PEA⁺ adsorption. The optimizations were performed at GGA/PBE+vdW level of theory;

FIG. 31C shows the optimized crystal structure for pristine PbI₂-rich (100) with NH₄⁺ and PEA⁺ adsorption. The optimizations were performed at GGA/PBE+vdW level of theory;

FIG. 31D shows the optimized crystal structure for defective PbI₂-rich (100)-V_{Pb} with NH₄⁺ and PEA⁺ adsorption. The optimizations were performed at GGA/PBE+vdW level of theory;

FIG. 32A shows the optimized crystal structure for pristine FAI-rich (110) with NH₄⁺ and PEA⁺ adsorption. The optimizations were performed at GGA/PBE+vdW level of theory;

FIG. 32B shows the optimized crystal structure for defective FAI-rich (110)-V_{FA} with NH₄⁺ and PEA⁺ adsorption. The optimizations were performed at GGA/PBE+vdW level of theory;

FIG. 32C shows the optimized crystal structure for pristine PbI₂-rich (110) with NH₄⁺ and PEA⁺ adsorption. The optimizations were performed at GGA/PBE+vdW level of theory;

FIG. 32D shows the optimized crystal structure for defective PbI₂-rich (110)-V_{Pb} with NH₄⁺ and PEA⁺ adsorption. The optimizations were performed at GGA/PBE+vdW level of theory;

FIG. 33A shows the optimized crystal structure for pristine FAI-rich (100) and defective FAI-rich (100)-V_{FA} with H⁺ adsorption;

FIG. 33B shows the optimized crystal structure for pristine PbI₂-rich (100) and defective PbI₂-rich (100)-V_{P}e with H⁺ adsorption;

FIG. 33C shows the optimized crystal structure for pristine FAI-rich (110) and defective FAI-rich (110)-V_{FA} with H⁺ adsorption;

FIG. 33D shows the optimized crystal structure for pristine PbI₂-rich (110) and defective PbI₂-rich (110)-V_{P}e with H⁺ adsorption;

FIG. 34 shows the calculated binding energies between H⁺ and pristine/defective FAPbI₃ (100) and (110) surfaces;

FIG. 35A shows the time-resolved PL (trPL) spectra of the perovskite films incorporated with different AQS derivatives;

FIG. 35B shows a table summarizing the parameters used for calculating the average carrier lifetime of I/Br mixed-halide perovskites in FIG. 35A;

FIG. 36A shows space-charge-limited current (SCLC) curves of the hole-only devices with the device structure of glass/ITO/DC-PA/perovskite (with and without AQS derivatives)/MoO₃/Ag);

FIG. 36B shows the corresponding calculated trap density in the perovskite films of FIG. 36A;

FIG. 37A shows the J-V curves of the control and target single-junction PSCs (perovskite with AQSP as additive). The initial efficiency of control and target single-junction PSCs are 18.08% and 18.89%, respectively;

FIG. 37B shows the J-V curves of the single-junction PSCs based on the perovskites with different concentrations of AQSP;

FIG. 37C shows the violin plots comparing the photovoltaic parameters of the control and target PSCs (20 devices fabricated from different batches);

FIG. 38 shows J-V curves of the target PSCs with reverse and forward scans;

FIG. 39A shows the J-V curves of the single-junction PSCs based on the perovskites with different concentrations of AQSP;

FIG. 39B is a table summarizing the solar cell performance for the single-junction PSCs under reverse scan (extracted from the J-V curves of FIG. 39A);

FIG. 40 shows the external quantum efficiency (EQE) curve with the determination of the bandgap for target PSC;

FIG. 41 shows the integrated J_{sc} from the corresponding EQE spectrum of the champion target PSC;

FIG. 42A shows the summary of the reported V_{oc} values for the representative PSCs with the bandgap ranging from 1.60 to 1.85 eV;

FIG. 42B is a table summarizing the reported V_{oc} values for the PSCs with the bandgap of approximately 1.80 eV corresponding to FIG. 42A;

FIG. 43 shows the continuous maximum power point (MPP) tracking of the encapsulated PSCs under simulated AM1.5G illumination (100 mW cm⁻², without UV filter) in an N₂-filled chamber without temperature control. The initial efficiency of control and target PSC are 18.08% and 18.98%, respectively;

FIG. 44 is a schematic diagram illustrating the structure of a monolithic (two-terminal) PO-TSC with an interconnection layer (ICL) of BCP/Au/MoO₃;

FIG. 45 shows the cross-section scanning electron microscopy (SEM) image corresponding to FIG. 44;

FIG. 46A shows the J-V curves of the PO-TSC under reverse and forward scans. The inset shows the stabilized power output (SPO) of the tandem cell;

FIG. 46B is a table summarizing the device performance of the champion PO-TSC under reverse and forward scans;

FIG. 47 shows the J-V curves of the PSC, OSC, and PO-TSC;

FIG. 48 shows the violin plots summarizing the photovoltaic parameters of PO-TSCs (25 devices fabricated from different batches);

FIG. 49 shows the EQE spectra of the perovskite and organic subcell in the PO-TSC. The dotted line indicates the sum of EQE % of the front subcell and rear subcell at different wavelengths. The dash line 1-R indicates the sum of transmittance and parasitic absorption of the device, with R being to the reflection of the whole device (i.e., the PO-TSC), which is measured from the glass/ITO side in an integrated sphere;

FIG. 50 shows the efficiency distribution of 25 individual tandem cells (from different batches);

FIG. 51 shows the summary of the PCEs for the reported PO-TSCs;

FIG. 52A to 52E show the certified results of the tandem solar cell measured at SIMIT (CNAS). The certified PCE is 24.3% with a certified aperture area of 0.0419 cm²;

FIG. 53 is table summarizing the device performance of the reported PO-TSCs and the PO-TSC of the present invention; and

FIG. 54 shows the continuous MPP tracking of the encapsulated PO-TSC under simulated AM1.5G illumination (100 mW cm⁻³², without UV filter) in an N₂-filled chamber without temperature control. The initial efficiency of the tandem cell is 24.67%.

### DETAILED DESCRIPTION OF OPTIONAL EMBODIMENT

As used herein, the forms “a”, “an”, and “the” are intended to include the singular and plural forms unless the context clearly indicates otherwise.

The words “example” or “exemplary” used in this invention are intended to serve as an example, instance, or illustration. Any aspect or design described in this disclosure as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances.

As used herein, the phrase “about” is intended to refer to a value that is slightly deviated from the value stated herein. In one example, the expression “about 10 seconds” encompasses any values from 9.5 seconds to 10.5 seconds, such as 9.5, 9.55, 9.85, 10.0, 10.02, 10.2 . . . 10.5 seconds. In another example, the expression “about 1 mol %” encompasses any value from 0.8 mol % to 1.2 mol % such as 0.8, 0.85, 0.88, 0.95 . . . 1 . . . 1.05 . . . 1.2 mol %. In still another example, the expression of “about 0.3 mol %” encompasses any value from 0.28 mol % to 0.32 mol %, such as 0.28, 0.285, 0.29, 0.292 . . . 0.3 . . . 0.305, 0.31 . . . 0.32 mol %. In a further example, the expression of “about 260 nm” encompasses any value from 255 nm to 265 nm, such as 255, 255.5, 256, 256.2 . . . 260 . . . 262, 263.5, 264 . . . 265 nm. In a still further example, the expression of “about 4000 rpm” encompasses any value from 3900 rpm to 4100 rpm, such as 3900, 3910, 3955 . . . 0.4000, 4005, 4050 . . . 4100 rpm.

The halide oxidation mechanism is considered as the one of the models that may rationalize the halide segregation behavior in perovskites. In particular, it is believed that upon illumination, preferential iodide oxidation occurs in I/Br mixed perovskites, leading to local concentration gradients of its oxidized products (e.g., I₂ and HI), which in turn drive halide migration. Furthermore, it is also believed that Pb²⁺ ions in perovskites are prone to be reduced to metallic Pb⁰ under continuous illumination, which is detrimental to device efficiency and stability.

Without intending to be limited by theory, the inventors have, through their own research, trials, and experiments, devised an active layer for solar cell, particularly a perovskite layer comprising a mixture of a halide perovskite and a sulfonyl naphthoquinone-based compound (or in other words a perovskite layer formed by mixing a halide perovskite and a sulfonyl naphthoquinone-based compound), that may be capable of effectively suppressing halide segregation of the active layer. In particular, in an example embodiment, it is found that the PSC equipped with the perovskite layer of the present invention may obtain a power conversion efficiency (PCE) of 19.58% with an open-circuit voltage (V_{oc}) of 1.351 V, while showing significantly improved long-term stability (T₉₅˜500 hours under maximum power point (MPP) tracking. In another example embodiment, the monolithic perovskite-organic tandem solar cell (PO-TSC) containing the perovskite layer of the present invention may have a PCE of 25.22% (certified 24.27%) and 92% of its initial PCE retained after operation for 500 hours.

According to the present invention, there is provided a perovskite layer for use in a solar cell comprising a mixture of a halide perovskite and a sulfonyl naphthoquinone-based compound having a structure of Formula (I):

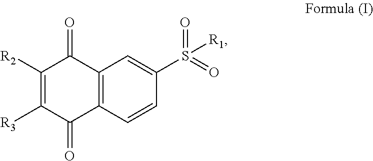

wherein:
R₁ is independently selected from one of OM, OR₄, and NR₅R₆, with M being a cation, R₄, R₅ and R₆ being independently selected from a hydrogen and a substituent; R₂ and R₃ are independently selected from a hydrogen and a substituent, or R₂ and R₃ may form a fused ring.

In an embodiment, M is selected from the group consisting of H⁺, Na⁺, K⁺, Cs⁺, Rb⁺, Sr²⁺, Ca²⁺, Ni²⁺, Cu²⁺, Co²⁺, Zn²⁺, Mg²⁺, Ba²⁺, Li⁺, ammonium ion, and aminum ion; R₂, R₃, R₄, R₅ and R₆ are independently selected from the group consisting of hydrogen, substituted or unsubstituted C1 to C4 alkyl, substituted or unsubstituted C2 to C6 alkenyl, substituted or unsubstituted C2 to C6 alkynyl, halogen, substituted or unsubstituted C6 to C10 aryl, —CN, —C(═O)OH, —C(═O)H, —C(═O)R₇, —OR₇, —SH, —SR₇, —NH₂, —NHR₆, —N(R₇)₂, —Si(R₇)₃, —OSi(R₇)₃, —S(O)OH and —P(O)(OH)₂ where R₇ is an alkyl or a phenyl; and when R₂ and R₃ form a fused ring, it comprises a substituted or unsubstituted 6-membered to 14-membered ring.

In an embodiment, the unsubstituted C1 to C4 alkyl may include methyl, ethyl, propyl, butyl, isopropyl, isobutyl, t-butyl, n-butyl and the like. In an embodiment, the unsubstituted C2 to C6 alkenyl may include ethenyl, 1-propenyl, 2-propenyl (allyl), isopropenyl, 2-methyl-1-propenyl, 1-butenyl, 2-butenyl and the like. In an embodiment, the unsubstituted C2 to C6 alkynyl may include ethynyl, propynyl, but-1-ynyl, but-2-ynyl, pent-1-ynyl, pent-2-ynyl, hex-1ynyl, hex-2ynyl, hex-3ynyl and the like. In an embodiment, the unsubstituted C6 to C10 aryl may include unsubstituted C6 to C10 homoaryl such as phenyl, naphthyl, tetrahydronaphthyl, indanyl and the like. In an embodiment, the unsubstituted C6 to C10 aryl may include unsubstituted C6 to C10 heteroaryl such as pyridyl, pyrazinyl, pyrimidinyl, pyridazinyl, quinolyl, isoquinolyl, benzothienyl, benzofuryl, indolyl, pyridine, pyrimidone, pyrazinone, pyrimidinone and the like.

In an embodiment, one or more hydrogen of the above substituents, i.e., C1 to C4 alkyl, C2 to C6 alkenyl, C2 to C6 alkynyl, and C6 to C10 aryl, may be substituted by one or more of the following moiety such as halogen (e.g. F, Cl, Br), —CN, —CF₃, alkenyl (e.g., —CH═CH₂, —CH═C(CH₃)₂, and the like), alkynyl (e.g., —C≡CH, —C≡CCH₃, and the like), —C(═O)OH, —C(═O)H, —C(═O)R₈, —OR₈, —SH, —SR₈, —NH₂, —NHR₈, —N(R₇)₈, —Si(R₈)₃, —OSi(R₈)₃, —S(O)OH and —P(O)(OH)₂, where R₈ is C1 to C4 alkyl as defined herein or a phenyl.

In an embodiment where R₂ and R₃ form a fused ring, it may comprise a unsubstituted 6-membered to 14-membered ring such as phenyl, anthracene, tetrahydronaphthyl, indanyl and the like or substituted 6-membered to 14-membered ring in which one or more hydrogen of the unsubstituted 6-membered to 14-membered ring is substituted with, for example, phenyl, carbonyl, primary amine, secondary amine and the like.

In an embodiment, the sulfonyl naphthoquinone-based compound may be any one of Formula (II), (III), (IV), (V), (VI), or (VII):

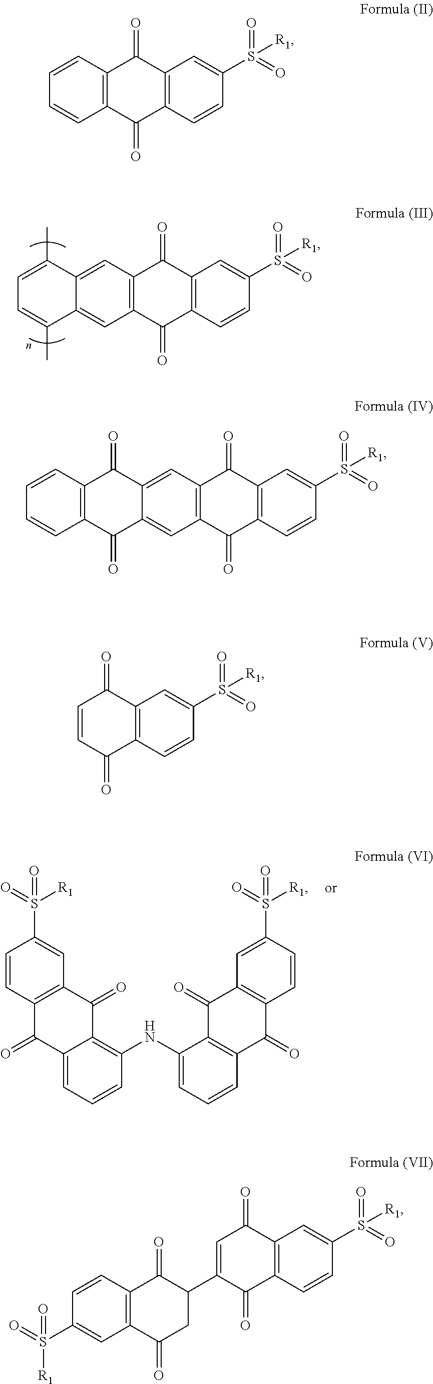

with R₁ as defined above; and n being 1-1000.

In a particular embodiment where the sulfonyl naphthoquinone-based compound has a structure of Formula (II), it may be selected from any one of Formula (VIII), Formula (IX), or Formula (X):

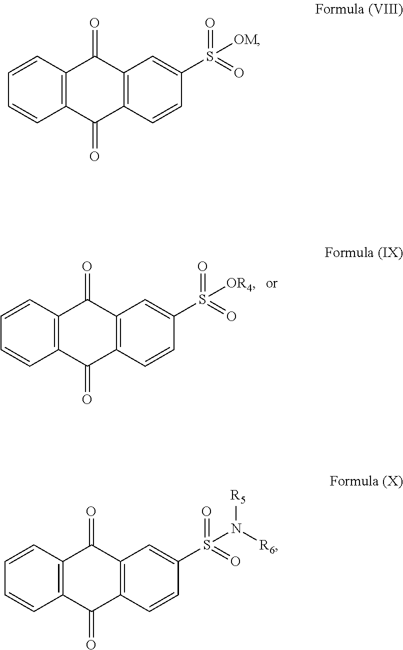

with M being H⁺, ammonium ion or aminum ion; R₄ being C1 to C4 alkyl; R₅ and R₆ being independently selected from hydrogen and C1 to C4 alkyl. The term “aminum ion” generally denotes a substituted ammonium ion formed by substituting one or more hydrogen atom in the ammonium ion with one or more organic groups such as an alkyl group (e.g. methyl, ethyl, propyl, phenylethyl, t-butyl, etc.), a benzyl group, a phenyl group and the like. C1 to C4 alkyl may include aliphatic hydrocarbons with straight chains and branched chains, and having 1 to 4 carbon atoms, such as methyl, ethyl, propyl, butyl, isopropyl, isobutyl, t-butyl, n-butyl and the like.

Preferably, the sulfonyl naphthoquinone-based compound of Formula (VIII) is selected from any one of Formula (VIIIa), (VIIIb), and (VIIIc):

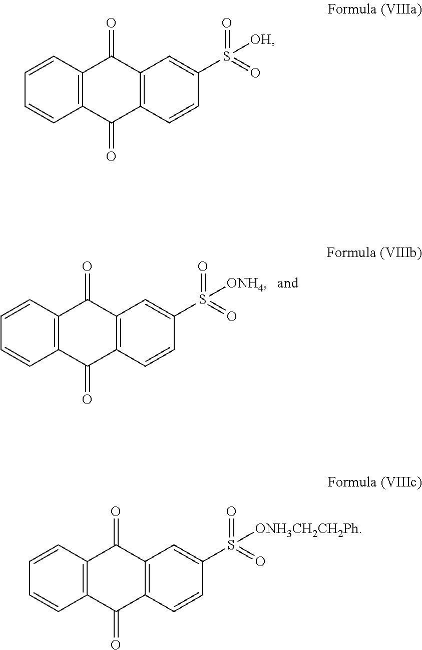

In particular, it is found that upon changing the cation M from H⁺to NH₄⁺ and phenylammonium (PEA) ion (PhCH₂CH₂NH₃⁺), it may enhance the passivation effect of the sulfonyl naphthoquinone-based compound, which in turn enhancing its ability to stabilize the perovskite upon operation, such as showing a stronger binding (e.g. higher binding energy) toward the redox components of the perovskite (e.g. Pb²⁺) upon operation. As a result, it may further facilitate the long-term operation of the perovskite. Details of the stabilization effect of the sulfonyl naphthoquinone-based compound will be discussed in the later part of the present disclosure.

In an optional embodiment, the sulfonyl naphthoquinone-based compound may comprise a structure of Formula (IXa):

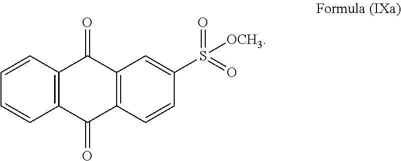 [SMILES: COS(=O)(=O)c1ccc2c(c1)C(=O)c1ccccc1C2=O]

In another optional embodiment, the sulfonyl naphthoquinone-based compound may comprise a structure of Formula (Xa):

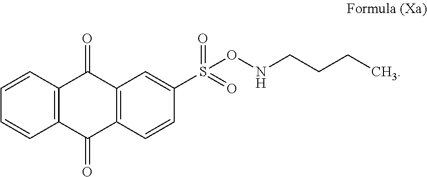 [SMILES: CCCCNOS(=O)(=O)c1ccc2c(c1)C(=O)c1ccccc1C2=O]

The halide perovskite of the perovskite layer may comprise a crystal grain structure of [A⁺¹B⁺²X⁻¹₃], with A⁺¹ being an A-site monovalent cation, B⁺² being a B-site divalent cation, and X⁻¹ being a halide anion.

In an embodiment, the A-site monovalent cation is selected from the group consisting of formamidinium (FA⁺), methylammonium (MA⁺), ethylammonium (EA⁺), guanidinium (GA⁺), Cs⁺, Rb⁺ and a combination thereof; the B-site divalent cation is selected from the group consisting of Pb²⁺, Sn²⁺, Ge²⁺ and a combination thereof; and the halide anion is selected from the group consisting of I⁻, Br⁻, Cl⁻ and a combination thereof.

In a particular embodiment, the halide perovskite is selected from any one of CsPb_{0.5}Sn_{0.5}I₃, FAPbI₃, MA_{0.25}FA_{0.75}PbI_{2.2}Br_{0.6}Cl_{0.2}, Cs_{0.02}FA_{0.96}MA_{0.02}PbI_{0.99}Cl_{0.01}, MAPB_{0.92}SN_{0.05}I₃, (FA_{0.95}MA_{0.05})_{0.95}CS_{0.05}PB(I_{0.96}BR_{0.04})₃, RB_{0.1}FA_{0.8}GA_{0.1}PB_{0.6}GE_{0.4}I₃, and (FA_{0.92}MA_{0.05})_{0.9}Cs_{0.1}Pb(I_{0.92}Br_{0.08})₃, and Cs_{0.2}FA_{0.8}Pb(I_{0.6}Br_{0.4})₃. Preferably, the halide perovskite may be an I/Br mixed perovskite. As a specific embodiment, the halide perovskite may be Cs_{0.2}FA_{0.8}Pb(I_{0.6}Br_{0.4})₃.

The term “crystal grain” (or crystallite) generally denotes small or microscopic crystals that are oriented/grown in random or no preferred direction, forming grain boundaries at areas/interfaces that the crystallites meet. In an embodiment, the crystal grain structure of [A⁺¹B⁺²X⁻¹₃] may include grain boundaries at which the sulfonyl naphthoquinone-based compound accommodates, and at which the sulfonyl naphthoquinone-based compound acts as a redox mediator which enables selective reduction of I⁰ and oxidation of Pb⁰ at the same time though electron shuttling.

The content/concentration of the sulfonyl naphthoquinone-based compound in the perovskite layer may vary/adjust in accordance with practical needs. In an embodiment, the perovskite layer may contain about 0.3 mol % to about 1 mol %, about 0.29 mol % to about 1.1 mol %, about 0.31 mol % to about 1 mol %, about 0.3 mol % to about 0.9 mol %, about 0.3 mol % to about 0.8 mol %, about 0.32 mol % to about 0.75 mol %, about 0.3 mol % to about 0.7 mol %, about 0.31 mol % to about 0.65 mol %, about 0.3 mol % to about 0.6 mol %, about 0.31 mol % to about 0.52 mol % or particularly about 0.3 mol % to about 0.5 mol % of the sulfonyl naphthoquinone-based compound.

In an additional or optional embodiment, the perovskite layer may further comprise about 3 mol % of MAPbCl₃ and 5 mol % of 4-guanidinobenzoic acid, which, in particular, act as passivating agents to (in addition to the activity of the redox mediator) further passivate the defect in the perovskite layer as discussed herein. Advantageously, it is appreciated that these passivating agents may be distributed at the grain boundaries of the perovskite layer without disturbing the chemical composition of the perovskite layer (i.e. without leading to substantial change of the chemical composition of the perovskite layer).

In an embodiment, the perovskite layer may have a thickness of about 200 nm to about 280 nm, about 205 nm to about 285 nm, about 210 nm to about 275 nm, about 220 nm to about 270 nm, about 220 nm to about 268 nm, about 240 nm to about 268 nm, about 255 nm to about 268 nm, about 258 nm to about 268 nm, or particularly about 260 nm.

The method for fabricating the perovskite layer as described herein is now disclosed. The method may comprise the steps of:
a) providing a solution mixture including a halide perovskite precursor and the sulfonyl naphthoquinone-based compound as described herein such as the sulfonyl naphthoquinone-based compound having a structure of Formula (VIII), Formula (IX), or Formula (X);
b) spin-coating the solution mixture on a substrate; and
c) annealing the spin-coated solution mixture to form the perovskite layer.

Step a) may comprises the steps of: a1) providing a first reaction mixture including halides of formamidinium, methylammonium, and cesium; and a2) mixing the first reaction mixture with about 0.3 mol % to about 1 mol % of the sulfonyl naphthoquinone-based compound to form a second reaction mixture.

For example, in an embodiment, the halides may comprise CsI, CsBr, FAI, FABr, PbI₂, and PbBr₂. These halides may be dissolved in a solvent mixture such as DMF/DMSO (e.g., v/v, 4:1) to form the first reaction mixture. Then, about 0.3 mol % to about 1 mol % of the sulfonyl naphthoquinone-based compound such as any one of the sulfonyl naphthoquinone-based compound having a structure of Formula (VIII), Formula (IX), or Formula (X) may be mixed with the first reaction mixture to form a second reaction mixture. Optionally or additionally, the second reaction mixture may be stirred for at least 8 hours to obtain a homogeneous/uniform solution mixture before proceeding to the spin coating step (i.e., step b)).

In an additional or optional embodiment, the first reaction mixture may further comprise about 3 mol % of MAPbCl₃ and 5 mol % of 4-guanidinobenzoic acid. For example, the MAPbCl₃ and 4-guanidinobenzoic acid may be added to the first reaction mixture comprising halides of formamidinium, methylammonium, and cesium before proceeding to step a2).

It is optional that the sulfonyl naphthoquinone-based compound may be obtained from commerce or by way of synthesis. In an optional embodiment where the sulfonyl naphthoquinone-based compound is obtained by way of synthesis, the method for fabricating the presently claimed perovskite layer may further comprise step a3) which involves converting sodium anthraquinone-2-sulfonate to the sulfonyl naphthoquinone-based compound as described herein.

In an embodiment, the sodium anthraquinone-2-sulfonate may be converted to the sulfonyl naphthoquinone-based compound having a structure of Formula (VIIa) by way of ion-exchange. For example, the sodium anthraquinone-2-sulfonate may be mixed with a aqueous solution of an cation exchange resin such as Amberlite cation exchange resin (IR-120 hydrogen form), followed by stirring for at least 8 hours. The mixture may be further subjected to isolation over a cation exchange column with the Amberlite cation exchange resin.

In another embodiment, the sodium anthraquinone-2-sulfonate may be converted to the sulfonyl naphthoquinone-based compound having a structure of Formula (VIIb) or Formula (VIIc). For example, the sodium anthraquinone-2-sulfonate may be converted to the sulfonyl naphthoquinone-based compound having a structure of Formula (VIIa) as described above. Then, the sulfonyl naphthoquinone-based compound of Formula (VIIa) may be converted to the sulfonyl naphthoquinone-based compounds of Formula (VIIb) and Formula (VIIb) by reacting the sulfonyl naphthoquinone-based compound of Formula (VIIa) with corresponding amine or ammonium salt, such as ammonium hydroxide and phenethylamine, and stirred for at least 8 hours, followed by drying under reduced pressure such as under vacuum.

In an optional embodiment, the sodium anthraquinone-2-sulfonate may be converted to anthraquinone-2-sulfonyl chloride, followed by further converting to the sulfonyl naphthoquinone-based compound having a structure of Formula (IXa) or Formula (Xa). For example, the sodium anthraquinone-2-sulfonate may be mixed with thioyl chloride in DMF to form a reaction mixture, followed by isolating the crude/raw anthraquinone-2-sulfonyl chloride from the reaction mixture by way of precipitation and filtration. The crude/raw anthraquinone-2-sulfonyl chloride may be optionally purified by washing with suitable solvent prior to further reaction.

For converting the anthraquinone-2-sulfonyl chloride to the sulfonyl naphthoquinone-based compound having a structure of Formula (IXa), for example, it may commence with mixing the anthraquinone-2-sulfonyl chloride with methanol under a solvent such as DMF, and stirring the mixture for, e.g., 2 hours at a temperature of about 50° C. to about 60° C., followed by isolating the crude/raw sulfonyl naphthoquinone-based compound of Formula (IXa) by way of precipitation and optionally filtration, and purification over column chromatography with an eluting solvent of, for instance, 0.5% methanol in dichloromethane (DCM).

On the hand, to convert the anthraquinone-2-sulfonyl chloride to the sulfonyl naphthoquinone-based compound having a structure of Formula (Xa), it may, for example, adding butylamine slowly (e.g. dropwise) to a DCM solution of the anthraquinone-2-sulfonyl chloride at a reduced temperature such as at 0° C. The reaction mixture may be stirred for, e.g., 2 hours at room temperature, followed by washing the crude/raw product with suitable solvent and purifying the crude/raw product over column chromatography with an eluting solvent of, for instance, 0.5% methanol in dichloromethane (DCM).

Turning to step b), the solution mixture (of step a)) may be spin-coated onto a substrate, such as a transparent substrate (e.g., glass, PDMS, PET, ITO, FTO, etc.), a hole transport layer, an electron transport layer and the like or a combination thereof at about 300 rpm to about 12000 rpm for about 10 seconds to about 140 seconds. In an embodiment, the spin-coating may be performed at about 4000 rpm to about 6500 rpm with a ramping rate of about 1500 rpm s⁻¹. During the spin-coating process and/or before the completion of the spin-coating process, such as prior 3 seconds to about 35 seconds of said completion, an anti-solvent agent may be added onto the spin-coated solution mixture. It is believed that the anti-solvent treatment would increase the nucleus density during the perovskite layer/film formation and thus is beneficial to produce uniform and pinhole-free perovskite layer/film. In an embodiment, the addition of anti-solvent agent may commence in step b1) in which an anti-solvent agent of chlorobenzene is added onto the center of the spin-coated solution mixture about 10 seconds before completing the spin-coating process.

Preferably, unless specified, it is appreciated that the spin coating processes as described herein are performed in an N₂-filled glovebox with the contents of O₂ and H₂O<5 ppm at a controlled temperature of about 20° C.

After that, the method may proceed to step c) annealing the spin-coated solution mixture to form the perovskite layer. For example, the spin-coated solution mixture (including the substrate) may be transferred to a hotplate and subjected to annealing at a temperature of about 65° C. to about 180° C., about 80° C. to about 180° C., about 80° C. to about 150° C., about 90° C. to about 150° C., about 95° C. to about 150° C., about 100° C. to about 120° C., about 100° C. to about 115° C., or about 100° C. to about 110° C. for about 6 min to about 120 min. It is believed that the annealing/heating process may be beneficial to the perovskite crystallization and consequently leading to higher power conversion efficiency upon (solar cell) operation. In an embodiment, the spin-coated solution mixture may be annealed at about 100° C. for about 15 min.

In an additional or optional embodiment, the method for fabricating the perovskite layer as described herein may further comprise the steps of: spin-coating a surface passivating agent of piperazinium iodide (PI) on the perovskite layer; and annealing the PI-coated perovskite layer. For example, a solution of PI such as an IPA (isopropyl alcohol) solution of PI may be spin-coated onto the perovskite layer as formed in step c), at a rate of about 5000 rpm for about 30 seconds. The PI-coated perovskite layer may then be annealed/heated at a temperature of about 100° C. for about 10 min. It is believed that the addition of PI may improve the band alignment and enhance charge extraction at the interface of the perovskite layer and the electron transport layer by creating a positive dipole, which may in turn enhancing the performance of the solar cell as a result.

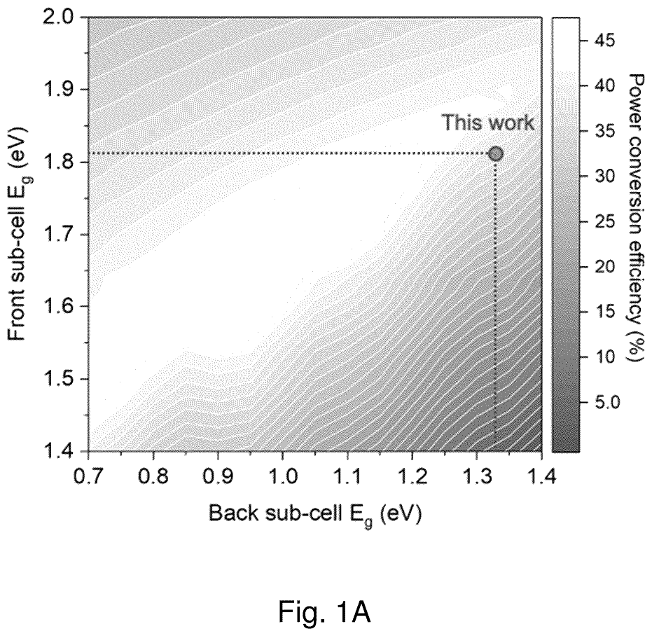

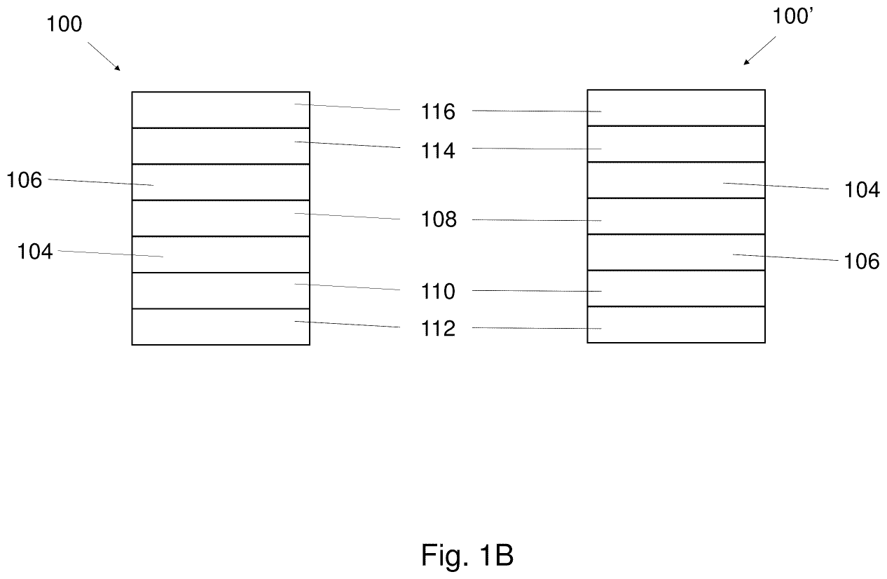

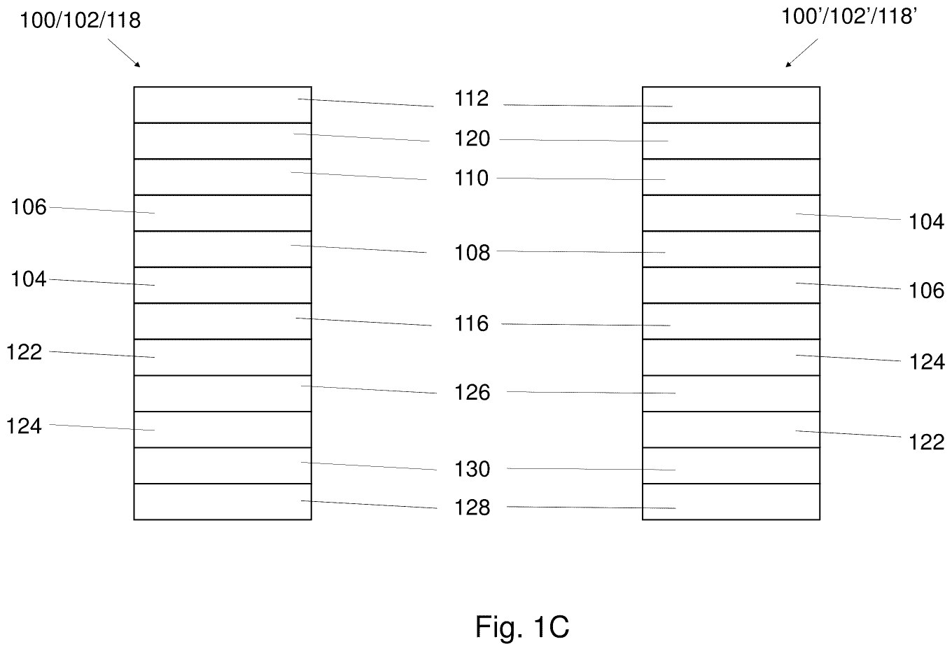

A further aspect of the present invention pertains to a solar cell, in particular a solar cell including the perovskite layer as described herein. In an embodiment, the solar cell may include: a first hole transport layer; a first electron transport layer; and a first active layer of the perovskite layer as described herein that is disposed between the first hole transport layer and the first electron transport layer, such as the solar cell 100 as exemplified in FIG. 1B.

As shown, the solar cell 100 has a first hole transport layer 104, a first electron transport layer 106, and a first active layer of the perovskite layer 108 as described herein, which is disposed between the first hole transport layer 104 and the first electron transport layer 106. In particular, the first active layer 108 is in direct contact with the first hole transport layer 104 and the first electron transport layer 106, forming a layered/stacked structure.

The first hole transport layer may be disposed on a transparent conductive layer that may be disposed on a transparent substrate and the first electron transport layer may be disposed on a first blocking layer that may be disposed on a first metal layer. For example, in this embodiment, the first hole transport layer 104 is in direct contact with the transport conductive layer 110 that is disposed on the transparent substrate 112, and the first electron transport layer 106 is in direct contact with the first blocking layer 114 that is disposed on the first metal layer 116 (FIG. 1B).

In an alternative embodiment, the solar cell 100 may be configured to have the position of the first hole transport layer 104 switched with the first electron transport layer 106, forming a solar cell 100′. That said, in this alternative embodiment, the first electron transport layer 106 is in direct contact with the transport conductive layer 110 that is disposed on the transparent substrate 112, and the first hole transport layer 104 is in direct contact with the first blocking layer 114 that is disposed on the first metal layer 116 (FIG. 1B).

As mentioned herein, the perovskite layer of the present invention is advantageous in enhancing the device (i.e., solar cell) efficiency and stability. For example, in an embodiment where the solar cell 100/100′ may be a perovskite solar cell such as a single-junction perovskite solar cell comprising a band gap of, e.g. about 1.81 eV, and may comprise a power conversion efficiency (PCE) of about 18.98% to about 19.58%. In another embodiment, the solar cell may comprise a power conversion efficiency of 95% of its initial value upon AM1.5G illumination for 500 hours at about 45° C. Detailed performance of the solar cell will be discussed in the later part of the present disclosure.

In an embodiment, the solar cell 100 may further comprise a subcell disposed thereon, forming a tandem solar cell. Referring to FIG. 1C, there is provided an exemplary configuration of a tandem solar cell 102. As shown, the tandem solar cell 102 includes the solar cell 100 and a subcell 118 disposed on the solar cell 100, in particular in direct contact with the first metal layer 116. In other words, in this embodiment, the solar cell 100 acts as a first subcell 102 in the tandem solar cell 102. The tandem solar cell 102 further includes an anti-reflection layer 120 to which the transparent conductive layer 110 and the transparent substrate 112 are disposed on and in direct contact therewith.

In this embodiment, the subcell 118 includes a second hole transport layer 122, a second electron transport layer 124, and a second active layer 126 that is disposed between, in particular, in direct contact with the second hole transport layer 122 and the second electron transport layer 124, forming a layered/stacked structure. The second hole transport layer 122 may be disposed on the first metal layer 116 and the second electron transport layer 124 may be disposed on a second metal layer 128.

In particular, it is appreciated that depending on the configuration of the solar cell 100, the second hole transport layer 122 and the second electron transport layer 124 may be configured differently. For example, in this embodiment, the second hole transport layer 122 is in direct contact with the first metal layer 116 and the second electron transport layer 124 is in direct contact with the second metal layer 128.

In an alternative embodiment where the tandem solar cell 102′ includes the solar cell 100′, the subcell may be a subcell 118′ in which the position of second hole transport layer 122 is switched with that of the second electron transport layer 124. That said, in this alternative embodiment, the second electron transport layer 124 is in direct contact with the first metal layer 116 and the second hole transport layer 122 is in direct contact with the second metal layer 128 (FIG. 1C).

In an optional or additional embodiment, the subcell 118/118′ may further include a second blocking layer 130 disposed between the second ETL 124/second HTL 122 and the second metal layer 128 (FIG. 1C). Preferably, the second blocking layer 130 is in direct contact with the second ETL 124/second HTL 122 and the second metal layer 128.

In an embodiment, the second active layer may be selected from the group consisting of a perovskite photovoltaic material, a Si photovoltaic material, a CIGS photovoltaic material, a CdTe photovoltaic material, an organic photovoltaic material and a combination thereof.

In an example embodiment, the second active layer may be made of an organic photovoltaic material such as PM6:Y6:P₇₁BM. In a particular example embodiment, the subcell which includes the second active layer of PM6:Y6:P₇₁BM may be coupled with a solar cell which includes a first active layer of the perovskite layer as described herein, such as Cs_{0.2}FA_{0.8}Pb(I_{0.6}Br_{0.4})₃ to form a perovskite-organic tandem solar cell.

In that particular embodiment, the tandem solar cell may have a power conversion efficiency of about 24.27% to about 25.22%, and a power conversion efficiency of 92% of its initial value upon AM1.5G illumination for 500 hours at about 45° C. Detailed performance of the tandem solar cell will be discussed in the later part of the present disclosure.

In some embodiments, the transparent substrate 112 may be flexible or rigid, and may have a light transmittance greater than about 80% (at 550 nm). In some particular embodiments, the transparent substrate may be selected from the group consisting of glass, polymethyl methacrylate (PMMA), polycarbonate (PC), general-purpose polystyrene (GPPS), polyethylene glycol terephthalate (PET), polyethylene naphthalate (PEN), polydimethylsiloxane (PDMS), styrene-ethylene-butylene-styrene (SEBS), ethylene terephthalateco-1,4-cylclohexylenedimethylene terephthalate (PETG), acrylonitrile butadiene styrene copolymers (ABS), polypropylene (PP), polyamide (PA), acrylonitrile-styrene copolymer (AS), and a combination thereof. In a more particular embodiment, the transparent substrate may be selected from any one of glass, PET, PEN, PDMS, SEBS, PP and a combination thereof. In a preferred embodiment, the transparent substrate may be selected from any one of glass, PET, PEN PDMS and a combination thereof. As a specific embodiment, the transparent substrate may be glass.

In some embodiments, the transparent conductive layer (TCL) 110 may preferably have a resistance of about 5 Ω sq⁻¹ to about 70 Ω sq⁻¹, for example, from about 5 Ω sq⁻¹ to about 20 Ω sq⁻¹, or from about 5 Ω sq⁻¹ to about 15 Ω sq⁻¹. It is believed that the resistance of less than about 5 Ω sq⁻¹ may affect the transparency of the conductive layer, whereas the resistance higher than about 70 Ω sq⁻¹ may affect the charge transfer of the devices. In particular, the TCL may be selected from the group consisting of Indium Tin Oxide (ITO), Aluminum Zinc Oxide (AZO), Fluorine Tin Oxide (FTO), graphene, poly(3,4-ethylenedioxythiophene):poly(styrene sulfonate) (PEDOT:PSS), Ag nanowire, Cu nanowire and a combination thereof. In an embodiment, the TCL may be selected from any one of ITO, FTO, PEDOT:PSS, and a combination thereof. In another embodiment, the TCL may be selected from any one of ITO and FTO. As a specific embodiment, the TCL may be ITO.

In some embodiments, the first hole transport layer (HTL) 104 and the second HTL 122 may be independently selected from the group consisting of poly(triaryl amine) (PTAA), PEDOT:PSS, NiOx, 2,2′,7,7′-Tetrakis[N,N-di(4-methoxyphenyl)amino]-9,9′-spirobifluorene (Spiro-OMeTAD), p-type self-assembled monolayer (DC-PA), MoOₓ and a combination thereof. In an embodiment, the first and the second HTL may be independently selected from any one of PTAA, NiOx, Spiro-OMeTAD, DC-PA, MoOₓ and a combination thereof.

In some embodiments, the first electron transport layer (ETL) 126 and the second ETL 124 may be independently selected from the group consisting of PC₆₁BM, C₆₀, SnO₂, PNDIT-F3N and a combination thereof.

In some embodiments, the first blocking layer 114 and the second blocking layer 130 may be independently selected from the group consisting of bathocuproine (BCP), bis-C₆₀, SnOₓ, Zr(acac)₂, MoOₓ and a combination thereof. In particular, it is appreciated that when the blocking layer is disposed on top of an ETL, it may be referred as a “hole blocking layer” which is intended to block minority charge carriers such as hole in this case, to cathode. The hole blocking layer may be selected from any one of BCP, Bis-C₆₀, SnOₓ, and Zr(acac)₂. In contrast, when the blocking layer is disposed on top of a HTL, it may be referred as an “electron blocking layer” which is intended to block minority charge carrier such as electron in this case to anode. The electron blocking layer may be MoOₓ.

In some embodiments, the anti-reflection layer 120 may be selected from the group consisting of MgF₂, LiF, PDMS and a combination thereof. In a particular embodiment, the anti-reflection layer may be selected from any one of MgF₂, PDMS and a combination thereof. As a specific embodiment, the anti-reflection layer may be MgF₂.

In some embodiments, the first metal layer 116 and the second metal layer 128 may be independently selected from the group consisting of Ag, Cu, Au, Al, W, Fe, Pt and a combination thereof. In particular, the first metal layer and the second metal layer may be independently selected from any one of Ag, Cu, Au, Al, and a combination thereof. It is appreciated that the metal layers may be referred as an electrode or a charge recombination layer depending on the position/configuration of the metal layer within the solar cell. For example, it is appreciated that when the metal layer is an intermediate layer connecting two solar cells or subcells, such as the first metal layer 116 in the tandem solar cell 102, such metal layer may be referred as a “charge recombination layer”. In contrast, when the metal layer is disposed/positioned/configured at the uppermost or lowermost of the solar cell, such as the first metal layer 116 in solar cell 100 and the second metal layer 128 in the tandem solar cell 102, such metal layer may be referred as an “electrode”.

The solar cell of the present invention may be fabricated by typical method such as the method as described below.

Typically, the fabrication of solar cell may include the steps of: providing a substrate; depositing the substrate with a transparent conductive layer; optionally cleaning and drying the deposited substrate; spin-coating a hole transport layer onto the transparent conductive layer; spin-coating an active layer onto the hole-transport layer; and thermally evaporating an electron-transport layer and a metal layer.

The substrate deposited with the transparent conductive layer, such as the substrate 112 deposited with the TCL 110, may be sequentially cleaned by sonication with detergent, deionized water, acetone, and isopropyl alcohol for about 15 min to about 90 min, respectively. Then, the substrate may be dried at about 40° C. to about 125° C. in oven for at least about 1 h. The cleaned and dry substrate may be treated with oxygen plasma for about 10 min to about 75 min and then transferred into a N₂-filled glovebox before subsequent spin-coating process.

The HTL such as the first HTL 104 and the second HTL 122 may be spin-coated onto the transparent conductive layer at about 1000 rpm to about 10000 rpm for 5 s to about 90 s, followed by annealing at about 75° C. to about 180° C. for about 5 min to about 90 min. It is believed that the spinning rate, times, and annealing temperature may affect the hole-transport layer's formation, thickness and conductivity. In some embodiments, the spin-coating may be performed at about 2000 rpm to about 8000 rpm for about 10 seconds to about 60 seconds, and the annealing temperature may be from about 80° C. to about 150° C.

The active layer such as the first active layer 108 and the second active layer 126 may be spin-coated onto the HTL at about 300 rpm to about 12000 rpm for about 10 s to about 140 s. Additionally or optionally, an anti-solvent agent may be slowly dripped onto the center of the spin-coated active layer at about 3 s to about 35 s before the end of spin-coating. The spin-coated active layer may then be transferred to the hotplate for annealing at about 65° C. to about 180° C. for about 6 min to about 120 min.

The ETL such as the first ETL 106 and the second ETL 124, the blocking layer such as the first blocking layer 114 and the second blocking layer 130, and the metal layer such as the first metal layer 116 and the second metal layer 130 may be deposited by thermal evaporation under high vacuum (e.g., <about 5×10⁻⁶ Torr). It is believed that a high vacuum condition is advantageous in achieving accurate thickness upon deposition. Preferably, the vacuum may be of at least about 1×10⁻⁵ Torr or less.

In an embodiment where the solar cell includes an anti-reflection layer such as the anti-reflection layer 120, the anti-reflection layer may be deposited onto the TCL by thermal evaporation.

Hereinafter, the present invention is described more specifically by way of examples, but the present invention is not limited thereto.

### EXAMPLES

### Materials and Reagents

The reagents and starting materials for the synthesis of 2-sulfonate anthraquinone (AQS) derivatives were commercially available and used without further purification, if not specified. Cesium iodide (CsI), formamidinium iodide (FAI), and formamidinium bromide (FABr) were purchased from Dysol (Australia). Lead iodide (PbI₂, purity of 99.999%), lead bromide (PbBr₂, purity of 99.9%), and 1-chloronaphthalene (1-CN) were purchased from TCI (Japan). Lead chloride (PbCl₂), methylammonium chloride (MACl), fullerene (C60), and bathocuproine (BCP, purity of 99.9%) were purchased from Xi'an Polymer Light Technology Corporation (China). N,N-dimethylformamide (DMF, purity of 99.99%), dimethyl sulfoxide (DMSO, purity of 99.50%), isopropanol (IPA, purity of 99.50%), and chlorobenzene (CB, purity of 99.90%) were purchased from J&K (China) and used as received. PM6 and Y6 were purchased from Solarmer Materials. PC₇₁BM was purchased from American Dye Source, Inc. Molybdenum oxide (MoOₓ), chloroform (CF, purity of 99.90%), and methanol (MeOH, purity of 99.90%) were purchased from Sigma-Aldrich. The gold and silver pellets for thermal evaporation use were purchased from commercial sources with high purity. ((2,7-dimethoxy-9H-carbazol-9-yl) methyl) phosphonic acid (DC-PA, hole-selective self-assembled monolayer) and piperazinium iodide (PI, surface passivating agent for perovskite) were synthesized as reported in our previous studies.

### Methods and Characterization

### Redox Mediator Experiments in Solution State

I⁰ (25 mg, 0.1 mmol) and Pb⁰ (25 mg, 0.12 mmol) powder dispersed in 2 mL mixed DMF/IPA solvent (volume ratio 1:10) with or without redox mediators (0.025 mmol), and the solutions were stirred at 100° C. for 60 min. The UV-vis absorption spectra of the upper solution (dilute to 1.0×10⁻⁵ M) and XRD patterns of the bottom precipitation from the sample and reference solutions (after 60 min) were measured. The representative solution and the absorption spectra of the bottom layer in which FAI mixed with mediator dissolved in DMF.

### Preparation of Perovskite Precursor

The wide-bandgap perovskite precursor (Cs_{0.2}FA_{0.8}Pb(I_{0.6}Br_{0.4})₃) with a concentration of 1.2 M was prepared by dissolving CsI (0.144 M), CsBr (0.096 M), FAI (0.576 M), FABr (0.384 M), PbI₂ (0.756 M), and PbBr₂ (0.48 M) in lmL mixed solvent of DMF/DMSO (v/v, 4:1). It should be noted that 3.0 mol % of MAPbCl₃ and 0.5 mol % of 4-guanidinobenzoic acid hydrochloride were added to the perovskite precursor upon device fabrication. These two chemicals may act as passivating agents to (in addition to the activity of the redox mediator) further passivate the defects in the perovskite layer as discussed herein. Advantageously, it is appreciated that these passivating agents may be distributed at the grain boundaries of the perovskite layer without disturbing the chemical composition of the perovskite layer. For the target devices (solar cells containing the redox mediators of as disclosed herein), 0.3 mol % of the redox mediator (i.e., AQSH, AQSN, or AQSP) was added to the perovskite precursor. The above solutions were stirred overnight at room temperature, and no filtration was required before use.

### Fabrication of Single-Junction Wide-Bandgap Perovskite Solar Cells (PSCs)

The pre-patterned indium-doped tin oxide (ITO) glass substrates were sequentially cleaned by sonication with detergent (Decon 90/deionized water with 1:1/v:v), deionized water, acetone, and isopropanol (IPA) for 15 min, respectively. Then, the cleaned ITO glass substrates were transferred into an oven at 100° C. for 24 hours and treated with O₂ plasma for 10 min before use. The DC-PA hole-selective SAM (0.75 mg mL⁻¹ in IPA) was spin-coated onto ITO glass substrates at 3,000 r.p.m. (with a ramping rate of 2,000 r.p.m. s⁻¹) for 25 s, and were subsequently annealed at 110° C. for 15 min. After cooling, the substrates were rinsed with IPA solvent and annealed for another 5 min at 100° C. The perovskite film was then deposited on the DC-PA layer by a one-step spin-coating method. Specifically, 50 μL of the perovskite precursor as described above (with or without the redox mediators) was spin-coated at 4,000-6,500 r.p.m. (with a ramping rate of 1,500 r.p.m. s⁻¹) for 30 s. During the spin-coating process, 200 μL of chlorobenzene (CB) anti-solvent was quickly dripped onto the center of perovskite film 10 s before the end of the process and then annealed at 100° C. for 15 min. Next, piperazinium iodide (PI) (0.3 mg mL⁻¹ in IPA) was dynamically spin-coated onto the as-formed perovskite at 5,000 r.p.m. for 30 s, followed by annealing at 100° C. for 10 min. All the above spin-coating processes were conducted in an N₂-filled glovebox with the contents of O₂ and H₂O<5 ppm at a controlled temperature of −20° C. Finally, 20 nm C60, 6 nm BCP, and 100 nm Ag were thermally evaporated in a high vacuum chamber (<2×10⁻⁶ Torr) through a metal shadow mask (aperture area: 0.0644 cm²), respectively. 100 nm of MgF₂ was thermally evaporated onto the glass side of the devices as an anti-reflection (AR) layer.

### Preparation of Organic Bulk Heterojunction (BHJ) Layer Precursor

PM6, Y6 and PC₇₁BM with a weight ratio of 1:0.96:0.24 were dissolved in chloroform (CF), while the concentration of PM6 was fixed at 7 mg mL⁻¹. The solvent additive (0.5 vol %), i.e., 1-CN, was added to the above solution. Then, the solution was stirred at 40° C. for 2 hours before use.

### Fabrication of Single-Junction Narrow-Bandgap Organic Solar Cells (OSCs)

The OSCs with p-i-n configuration were based on the device structure of glass/ITO/MoOₓ/PM6:Y6:PC₇₁BM/PNDIT-F3N/Ag. First, 10 nm MoOₓ was thermally evaporated (with a rate of 0.05 Å s⁻¹) on clean ITO substrates in a high vacuum chamber (<6×10⁻⁷ Torr). Then, PM6:Y6:PC₇₁BM solution was dynamically cast onto MoOₓ layer at 1,500 r.p.m for 40 s, followed by thermal annealing at 90° C. for 10 min. After cooling, PNDIT-F3N (0.5 mg mL⁻¹ in methanol with 0.5 vol % of acetic acid) interfacial layer was dynamically spin-coated on the organic BHJ layer at 1,500 r.p.m. for 40 s. Finally, 100 nm Ag was thermally evaporated in a high vacuum chamber (<2×10⁻⁶ Torr) through a metal shadow mask (aperture area: 0.0644 cm²).

### Fabrication of Perovskite-Organic Tandem Solar Cells (PO-TSCs)

For the monolithic (two-terminal) PO-TSCs in this work, the narrow-bandgap organic subcells were integrated on top of the perovskite sub-cells. Briefly, after the thermal evaporation of BCP in wide-bandgap sub-cells, 0.5 nm Au (with a rate of 0.05 Å s⁻¹) and 10 nm MoOₓ were thermally evaporated on top of BCP, respectively, to form an ICL structure for the tandem cells. Then, the organic BHJ layer and interfacial layer (i.e., ICL) were sequentially spin-coated on MoOₓ. PNDIT-F3N was dynamically spin-coated on the organic BHJ layer at 1,500 r.p.m. for 40 s. Finally, 100 nm Ag was thermally evaporated through a metal shadow mask (aperture area: 0.0644 cm²).

### Methods and Characterizations

¹H NMR and ¹³C NMR spectra were measured on Bruker AVANCE III 300 MHz and 400 MHz spectrometers. Solution UV-Vis absorption spectra were obtained from Agient8454 spectrophotometer. Cyclic voltammetry (CV) measurements were conducted on a CHI660D electrochemical workstation. The CV experiments were performed at room temperature with a conventional three-electrode system using a glassy carbon electrode as the working electrode, Pt wire as the counter electrode, and Ag/AgCl (saturated KCl) as the reference electrode. Ammonium chloride (NH₄Cl, 0.1M) in an aqueous solution was used as the supporting electrolyte, and the scan rate was 0.1 V s⁻¹. Elemental analyses were obtained by Elemantar: Vario UNICUBE. Transmittance and absorption spectra were conducted on an ultraviolet-visible (UV-vis) spectrometer (PE Lamda 750).

Time-dependent photoluminescence (tdPL) spectra were collected by a home-built facility, where an excitation laser (450 nm) was introduced to the sample through a fiber, and the PL spectra were detected by using a detector connected with an Ocean Optics USB2000. X-ray photoelectron spectroscopy (XPS) was performed on Thermo Fisher ESCALAB XI+ X-ray Photoelectron Spectrometer. The non-monochromatic He I is employed as UV light with an energy of 21.21 eV.

The top-viewing morphology of the thin film samples, cross-section profile of tandem cell, and the cross-sectional energy dispersive spectroscopy (EDX, line scan) were conducted by scanning electron microscopy (SEM, QUATTRO S). Powder and thin film X-ray diffraction (XRD) characterizations were conducted on D2 Phaser instrument with a Cu Kα (a wavelength of 1.5418 Å) radiation.

Time-resolved photoluminescence spectrum (trPL) was recorded by FLS980 spectrofluorometer (Edinburgh) with a 480 nm pulsed excitation laser. The current density-voltage (J-V) characteristics of the devices were measured in an N₂-filled glovebox at room temperature using a Keithley 2400 SourceMeter under simulated sunlight from a solar simulator (EnliTech, SS-F5, Taiwan). A National Renewable Energy Laboratory (NREL) calibrated silicon solar cell (with a KG-2 filter) was used to obtain AM 1.5G (100 mW cm⁻³²) solar simulator's light intensity. The perovskite solar cells were covered with a shading mask with an aperture area of 0.0419 cm² to ensure the accuracy of current density from J-V curves.

The J-V measurements were conducted with sweep mode with reverse and forward scans with a scan rate of 10 mV s⁻¹ and a step of 0.02 V. The EQE curves were carried out by an EQE measurement system (EnliTech, QE-R, Taiwan). For perovskite-organic tandem solar cells, the EQE of wide-E_{g} perovskite subcells was measured while saturating the corresponding organic subcells by applying a bias illumination with an 800 nm long-pass optical filter. Likewise, the EQE of narrow-E_{g} organic subcells was measured while saturating the corresponding perovskite subcells by applying a bias illumination with a 500 nm short-pass optical filter. No bias voltage was applied during all EQE measurements.

### Long-Term Device Stability Measurement

The long-term device stability of the encapsulated devices was tested by an in-situ stability measurement system (CRYSCO) equipped with multiple sample chambers (without UV filter and temperature control) and automatic data collecting elements. The solar cells were tracked at their maximum power point (MPP) under a simulated AM 1.5G spectrum (with 1-sun equivalent intensity). The light source is a SLED lamp with the wavelength range from 400 to 1,000 nm. A silicon photoelectric probe was equipped to monitor the change in the light intensity and adjust the intensity automatically with the control of the host. The sample chamber was placed in the air with a continuous N₂ flow during the test.

### Density Functional Theory (DFT) Calculations

The DFT calculations were carried out using the projector-augmented wave (PAW) method as implemented in the Vienna Ab initio Simulation Package (VASP) code. The generalized gradient approximation (GGA) together with the Perdew-Burke-Ernzerhof (PBE) exchange-correlation functional was applied. The van der Waals (vdW) interactions were also included in the calculations using the zero damping DFT-D3 method of Grimme. A uniform grid of 6×6×6 k-mesh in the Brillouin zone was employed to optimize the crystal structures of cubic-phase FAPbI₃ in bulk, 4×4×1 k-mesh for FAPbI₃ slabs, and 2×2×1 k-mesh for molecule/FAPbI₃ interfaces. The energy cutoffs of the wavefunctions were set at 500 eV for the bulk and 450 eV for the slabs and interfaces. The unit cells had a (2×2) lateral periodicity and contained four or five octahedral layers of FAPbI₃ with exposed (100) and (110) surfaces with different terminations (FAI-rich and PbI₂-rich) and surface defects (V_{FA} and V_{Pb}). The slab replicas were separated by ˜20 Å of vacuum. Each structure was optimized until forces on single atoms were smaller than 0.015 eV A¹. The energy barriers for the half-reactions related to the elimination of Pb⁰ and I⁰ were calculated as E (Pb⁰ elimination)=E (AQS²⁻+Pb²⁺)−E (AQS)+E (Pb⁰) and E (I⁰ elimination)=E (AQS⁻+I⁻)−E (AQS²⁻)+E (I⁰). The binding energy was calculated as E (binding)=E (interface)−E (FAPbI₃)−E (molecule), where E (interface) is the total energy of FAPbI₃ upon molecular adsorption, and E (FAPbI₃) and E (molecule) are the energies of isolated FAPbI₃ and PEA⁺ [H⁺ or NH₄⁺].

### Example 1A

### Synthesis of AQSH

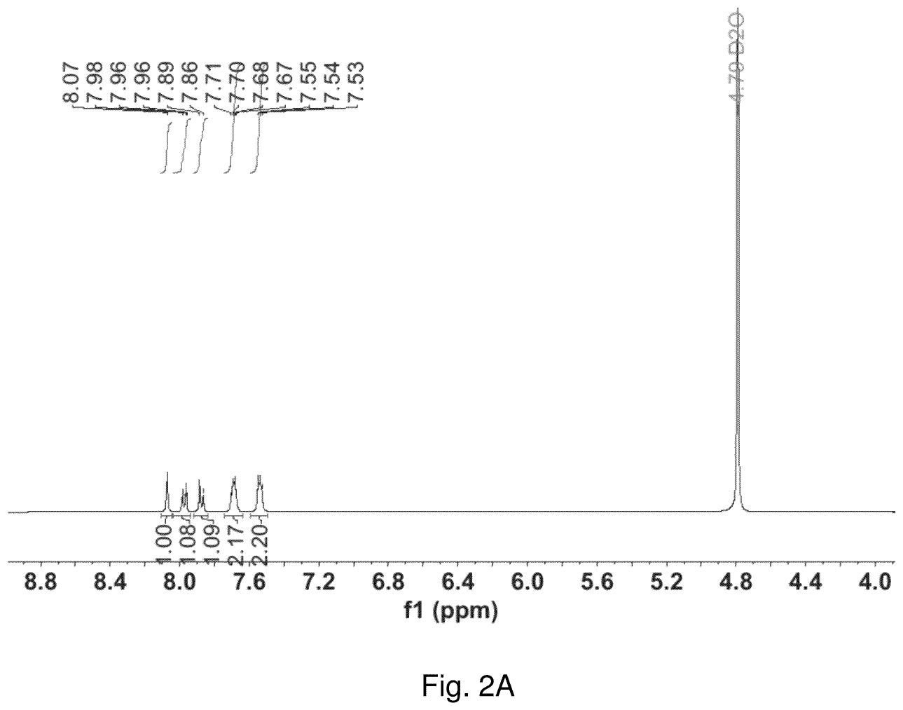

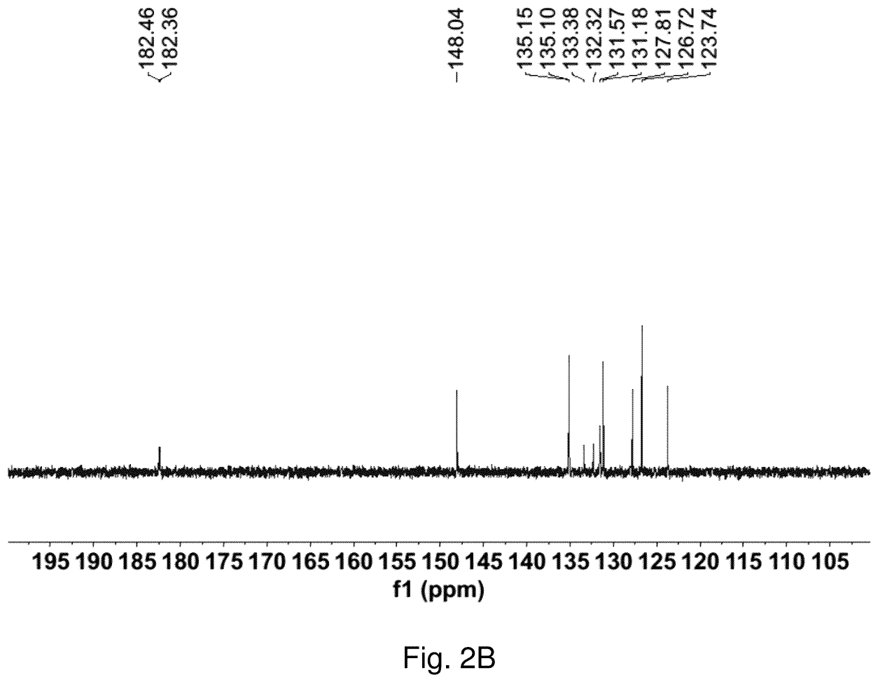

Sodium anthraquinone-2-sulfonate (300 mg, 0.97 mmol) and 3 mL Amberlite cation exchange resin (IR-120 hydrogen form) were added to 5 mL deionized water. The mixture was stirred overnight when the anthraquinone compound was dissolved. Then the solution was flushed over a cation exchange column with Amberlite cation exchange resin again. The obtained sulfonic acid was evaporated, and the compound was dried under a vacuum overnight. The product was obtained as pale-yellow powder with a yield of 216 mg (yield: 77%). ¹H NMR (400 MHz, D₂O) δ 8.07 (s, 1H), 7.97 (d, J=8.2 Hz, 1H), 7.88 (d, J=8.2 Hz, 1H), 7.74-7.64 (m, 2H), 7.58-7.50 (m, 2H) (FIG. 2A). ¹³C NMR (101 MHz, D₂O) δ 182.46, 182.36, 148.04, 135.15, 135.10, 133.38, 132.32, 131.57, 131.18, 127.81, 126.72, 123.74. (two peaks overlap) (FIG. 2B).

### Example 1B

### Synthesis of AQSN

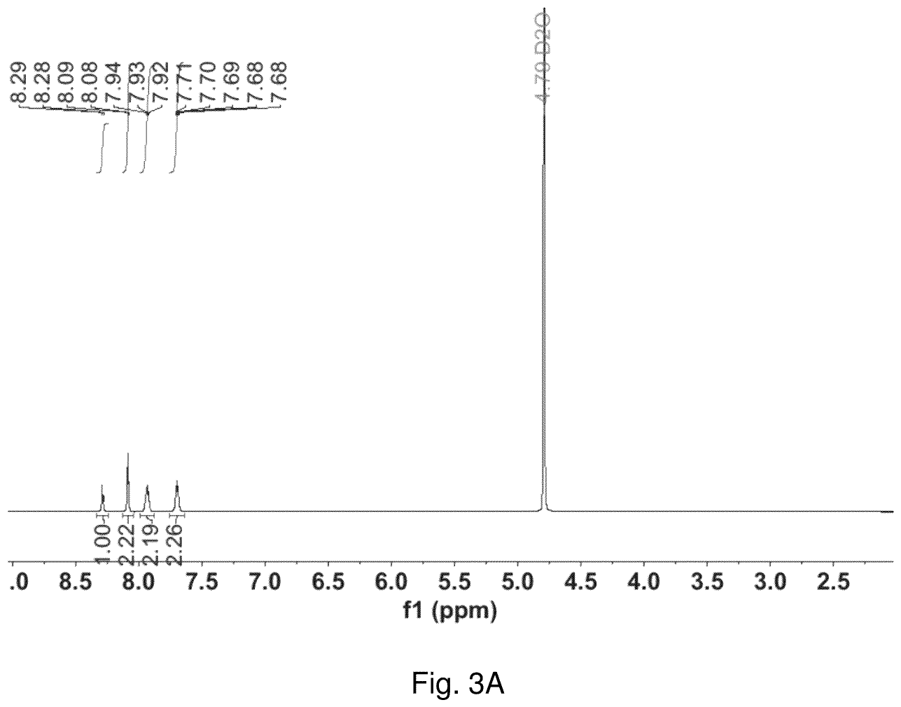

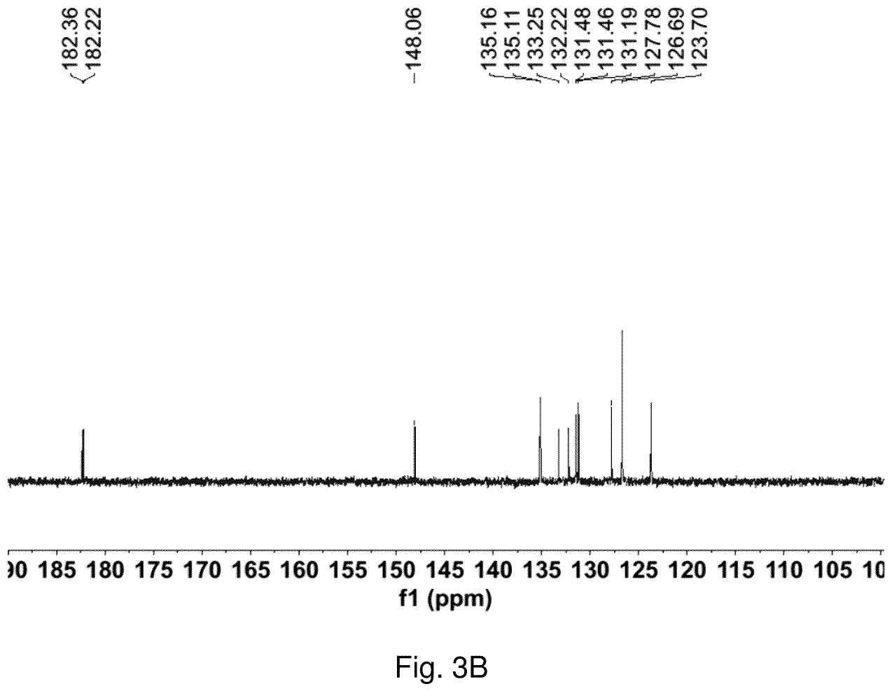

An aqueous solution of AQSH (0.97 mmol) obtained as mentioned in Example 1A was cooled to 0° C., and then 0.5 mL 30% ammonium hydroxide was added to convert the acid to ammonium salt. The water was evaporated and the compound was dried under vacuum overnight. The product was obtained as light-yellow powder with a yield of 278 mg (yield: 94%). ¹H NMR (400 MHz, D₂O) δ 8.28 (s, 1H), 8.14-8.04 (m, 2H), 8.02-7.87 (m, 2H), 7.76-7.61 (m, 2H) (FIG. 3A). ¹³C NMR (101 MHz, D₂O) δ 182.36, 182.22, 148.06, 135.16, 135.11, 133.25, 132.22, 131.48, 131.46, 131.19, 127.78, 126.69, 123.70. (one peak overlap) (FIG. 3B). Anal. Calcd for C14H₁₁NO₅S: C 55.08%, H 3.63%, N 4.59%, S 10.50%; found: C 54.52%, H 3.63%, N 4.57%, S 10.94%.

### Example 1C

### Synthesis of AQSP

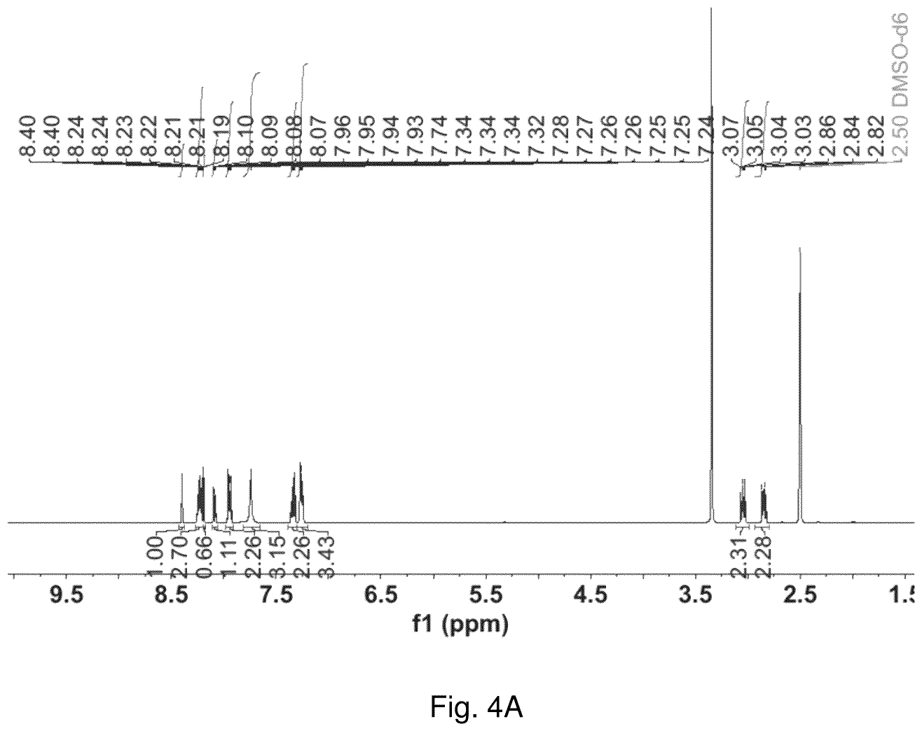

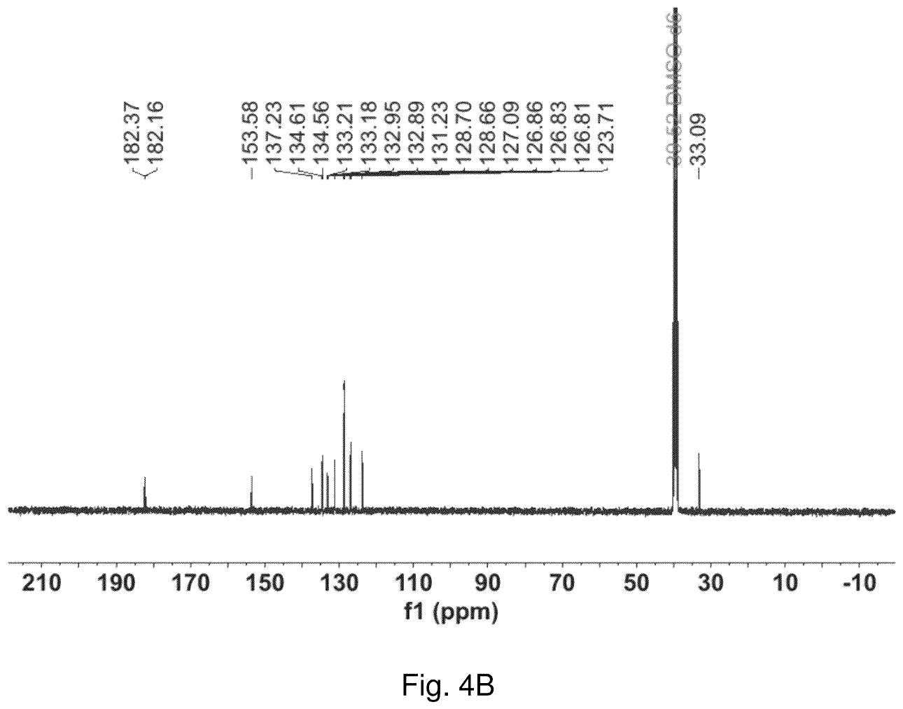

An aqueous solution of AQSH (0.97 mmol) obtained as mentioned in Example 1A above was cooled to 0° C. and then 1.2 equiv. phenethylamine was added to convert the acid to ammonium salt. The water was evaporated and the compound was dried under vacuum overnight. The product was obtained as light-yellow powder with a yield of 311 mg (yield: 76%). ¹H NMR (400 MHz, DMSO-d₆) δ 8.40 (d, J=1.7 Hz, 1H), 8.29-8.16 (m, 3H), 8.09 (dd, J=8.1, 1.7 Hz, 1H), 7.94 (dq, J=7.3, 4.0 Hz, 2H), 7.75 (s, 3H), 7.40-7.29 (m, 2H), 7.29-7.19 (m, 3H), 3.04 (t, J=9.6 Hz, 2H), 2.84 (t, J=9.6 Hz, 2H) (FIG. 4A). ¹³C NMR (101 MHz, DMSO-d₆) δ 182.37, 182.16, 153.58, 137.23, 134.61, 134.56, 133.21, 133.18, 132.95, 132.89, 131.23, 128.70, 128.66, 127.09, 126.86, 126.83, 126.82, 123.72, 33.09. (one peak overlap) (FIG. 4B). Anal. Calcd for C22H₁₉NO₅S: C 64.53%, H 4.68%, N 3.42%, S 7.83%; found: C 64.60%, H 4.68%, N 3.29%, S 8.18%.

### Example 2

### Synthesis of AQSester

Sodium anthraquinone-2-sulfonate (2.5 g, 8 mmol) was first converted to the corresponding sulfonyl chloride by reacting with thionyl chloride (10 mL, 137 mmol) and DMF (0.5 mL). The reaction mixture was poured into ice water. The precipitate was filtered and dried under vacuum to yield a yellow solid (2.4 g, 98%). To a mixture of sulfonyl chloride (1 mmol) and methanol (1 mmol) in DMF (3 mL) was added pyridine (0.06 mL) and the mixture was stirred at 50-60° C. for 2 h. The solution was cooled to room temperature and poured into water (20 mL). The resulting precipitate was filtered, washed with water and purified by column chromatography (0.5% MeOH in DCM) to yield a pale yellow solid with 195 mg (yield: 65%).

### Example 3

### Synthesis of AQamide

Butylamine (0.16 mL, 1.6 mmol) was added dropwise to a solution of sulfonyl chloride (100 mg, 0.33 mmol) in DCM (5 mL) at 0° C. The reaction mixture was allowed to warm to room temperature and stirred for 2 h. The reaction mixture was washed with water and purified by column chromatography (0.5% MeOH in DCM) to yield a pale yellow solid of 105 mg (yield: 92%).

### Example 4

### Design and Characterization

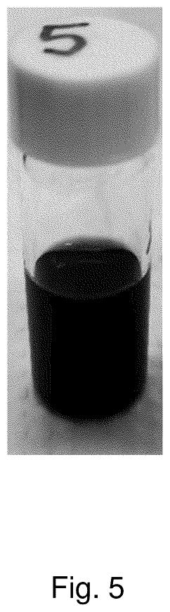

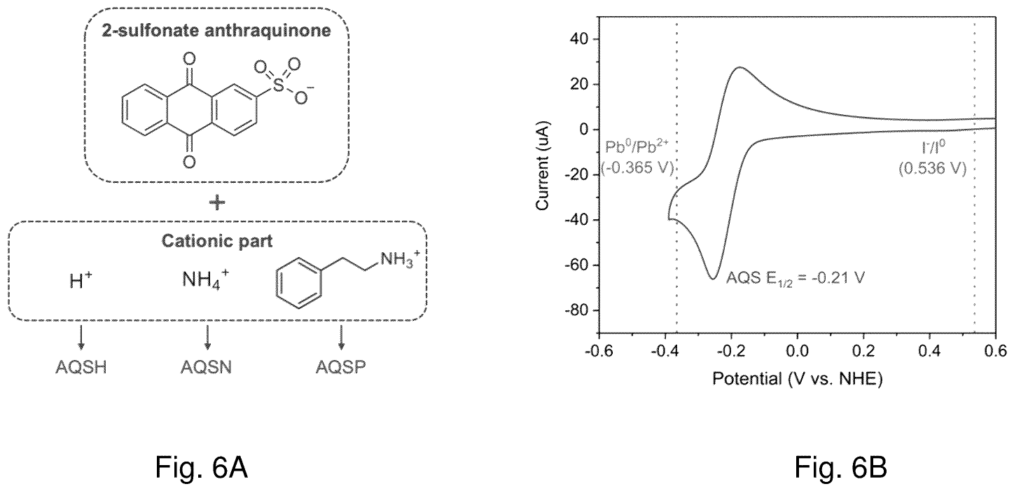

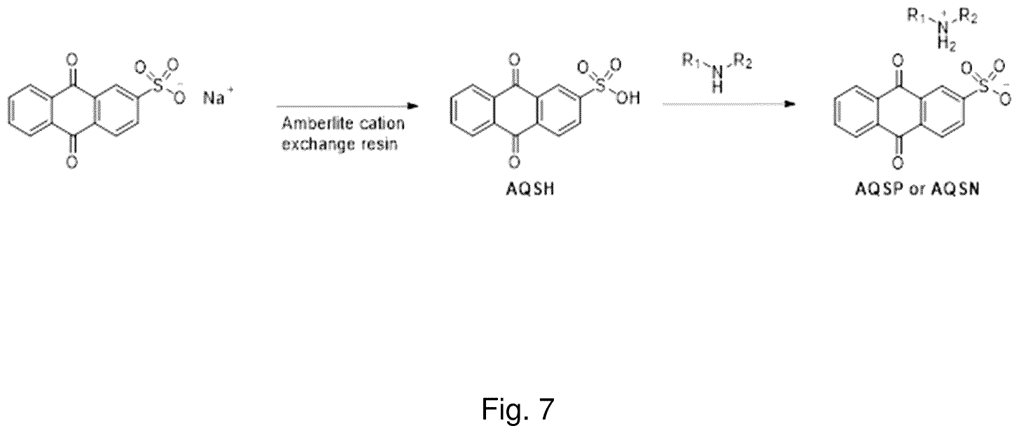

While it is believed that anthraquinone (AQ) may be used as an redox scaffold, the inventors through their own researches and experiments revealed that AQ in fact could not facilitate the formation of PbI₂ through electron shuttling between Pb⁰ and I⁰, which may be attributed to its relatively low redox potential compared to Pb, rendering its incapability in oxidizing metallic Pb⁰ (FIG. 5). To resolve this, the inventors devised that the AQ scaffold may be functionalized with a strong electron-withdrawing —SO₃⁻ group to tailor its redox potential to reach −0.21 V vs. NHE, enabling suitable redox shuttling between Pb (−0.365 V vs. NHE) and I₂ (0.536 V vs. NHE). This modification also increased its solubility in polar organic solvents (i.e., DMF and DMSO) for better processability and provided additional synthetic tunability for its properties (FIGS. 6A and 6B). Furthermore, it is found that by substitution of —H with —NH₄ and —PEA in the cationic part of AQS (FIG. 7), it may impart the molecules with defect passivation capability.

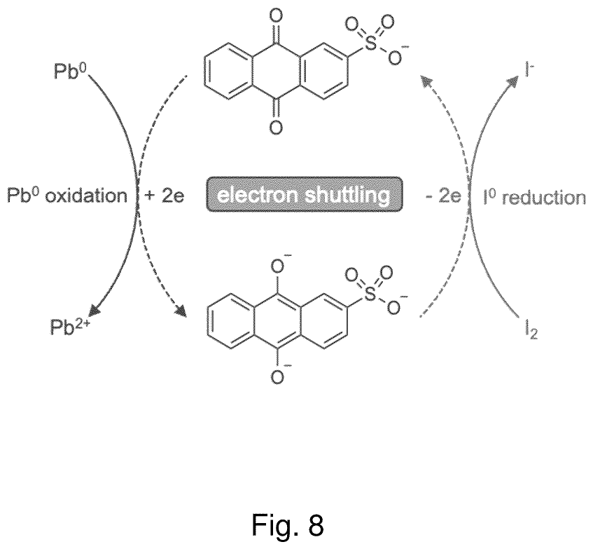

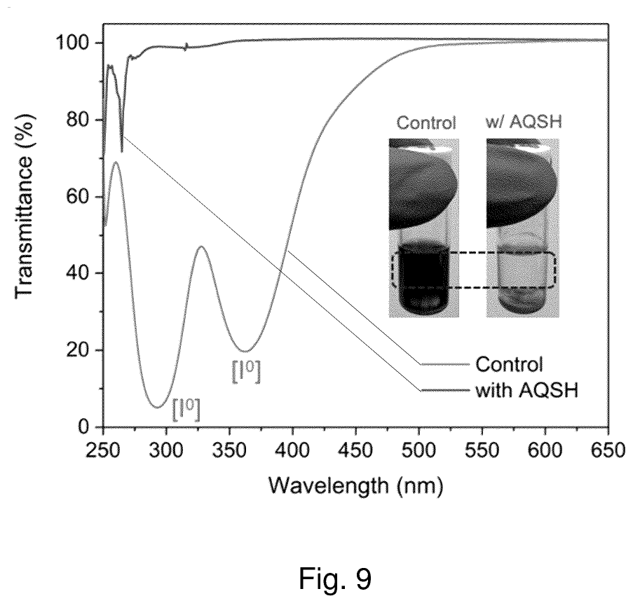

The proposed redox shuttle is illustrated in FIG. 8, where AQS core governs the redox ability of the molecules. The feasibility of electron transfer between Pb⁰ and I⁰ was first examined by dissolving AQSH, Pb⁰, and I⁰ powders into a DMF:IPA mixed solvent (with a volume ratio of 1:10) to provide a good trade-off between the solubility of the raw powders and precipitation of as-formed PbI₂ (FIG. 9). The control sample (w/o AQSH), shown in the inset of FIG. 9, exhibited minimal yellow solid products and remained deep brown. In contrast, the target sample containing AQSH turned colorless with many yellow precipitates formed, demonstrating efficient conversion of Pb⁰ and I⁰ to PbI₂ solid through electron shuttling. The transmittance spectra of the supernatant solution (taken from the samples) further confirmed the presence of I⁰ species only in the control solution, as two characteristic peaks at ˜290 nm and ˜360 nm were recorded.

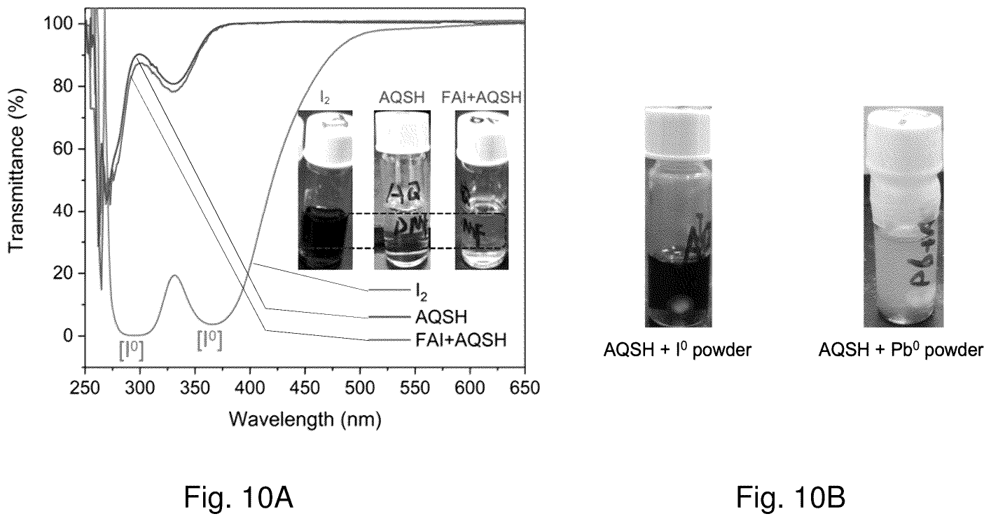

To exclude the possibility of AQSH oxidizing iodides, another set of solutions in DMF consisting of pure I₂, pure AQSH, and FAI:AQSH were prepared, respectively (FIG. 10A). The transparent FAI:AQSH solution displayed no peaks from I⁰ species, implying the selective oxidation of Pb⁰ instead of I⁻. The solutions consisted of AQSH with Pb⁰ or I⁰ were also tested to further verify this selective redox process (FIG. 10B).

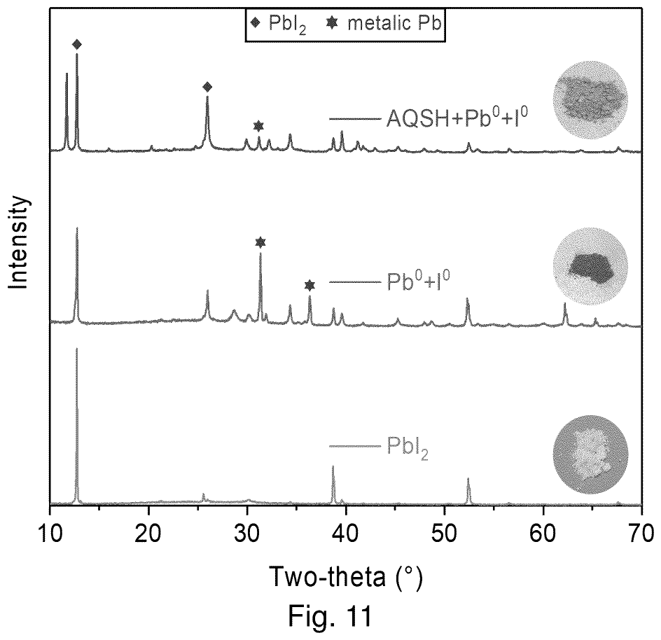

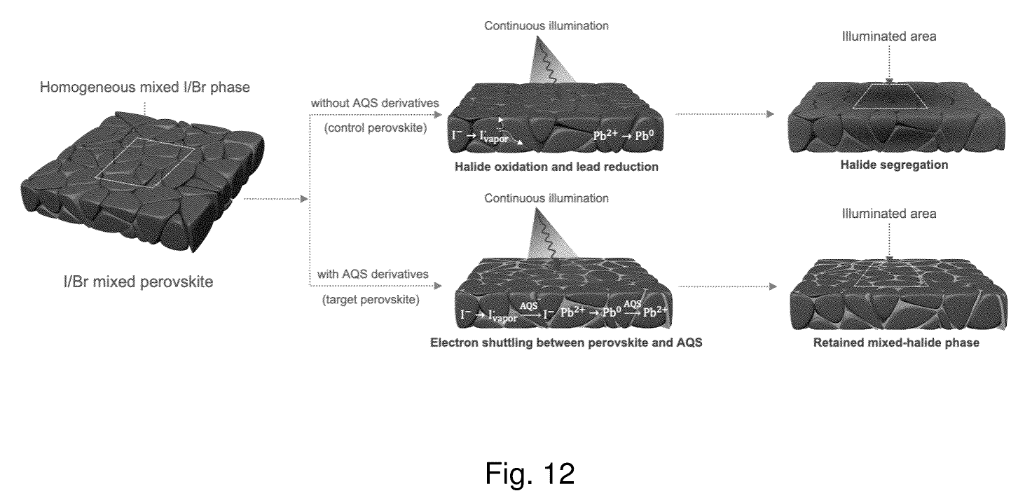

By performing powder X-ray diffraction (XRD), the majority of yellow precipitates (the inset in FIG. 9) were verified to be PbI₂, based on the presence of the characteristic diffraction peaks at 12.7°, 26.0°, and 38.7° (FIG. 11). Therefore, it is speculated that the AQS derivatives would act as an efficient redox mediator for perovskite, enabling the selective reduction of I⁰ and oxidation of Pb⁰. The simultaneous elimination of these defects should help suppress halide segregation in mixed-halide perovskites (as depicted in FIG. 12).

### Example 5

### Suppression of Halide Segregation in Mixed-Halide Perovskites

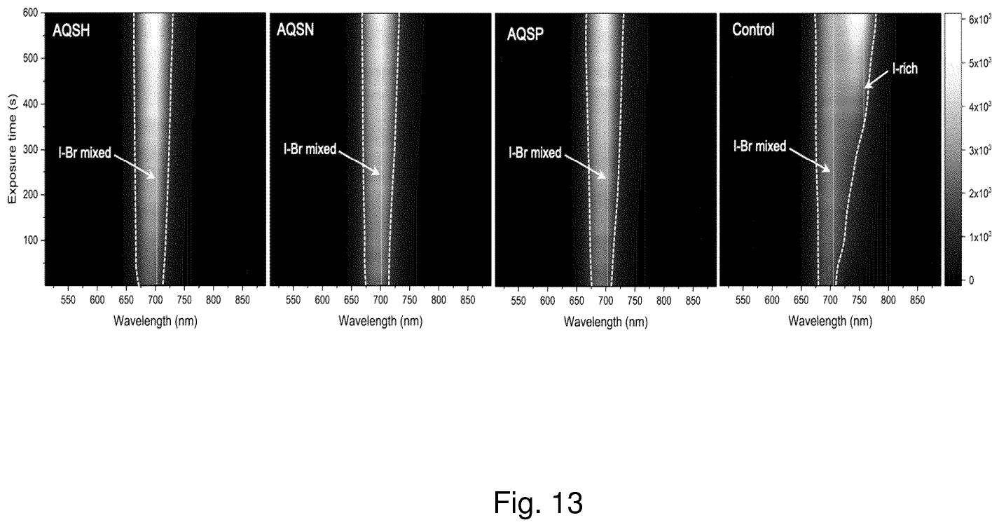

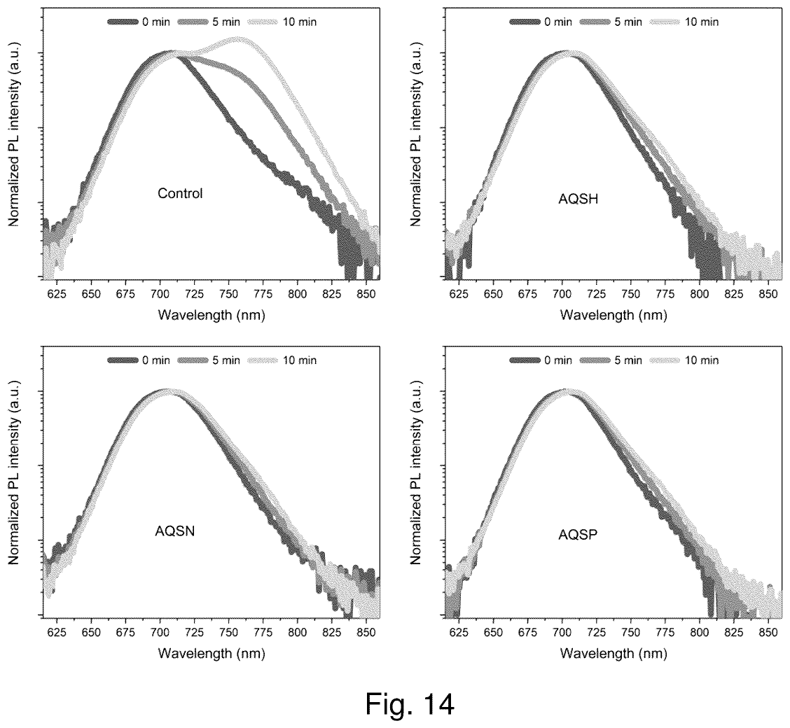

The preferential oxidation of the halide with a relatively low oxidation potential (I⁻<Br⁻<Cl⁻) is the critical step for halide mass transport, which ultimately triggers phase segregation in perovskites. Therefore, time-dependent PL (tdPL) was first conducted to investigate the effect of AQS-based redox mediators on halide segregation (FIG. 13). The AQS derivatives, namely AQSH, AQSN, and AQSP, were incorporated as additives respectively for I/Br mixed perovskite films. The initial PL peaks at ˜700 nm were assigned to the pristine perovskite. After continuous illumination, the PL of the AQS-based perovskite films remained stable, exhibiting only a slight red shift by 3-5 nm (FIG. 14). In contrast, a double-peak shape, which is the sign of halide segregation, gradually appeared in the PL spectrum of the control sample within a few minutes.

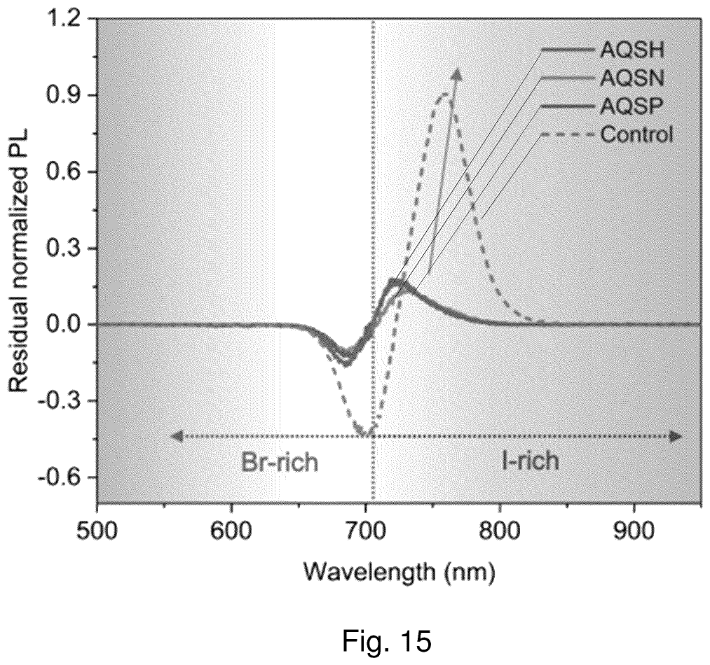

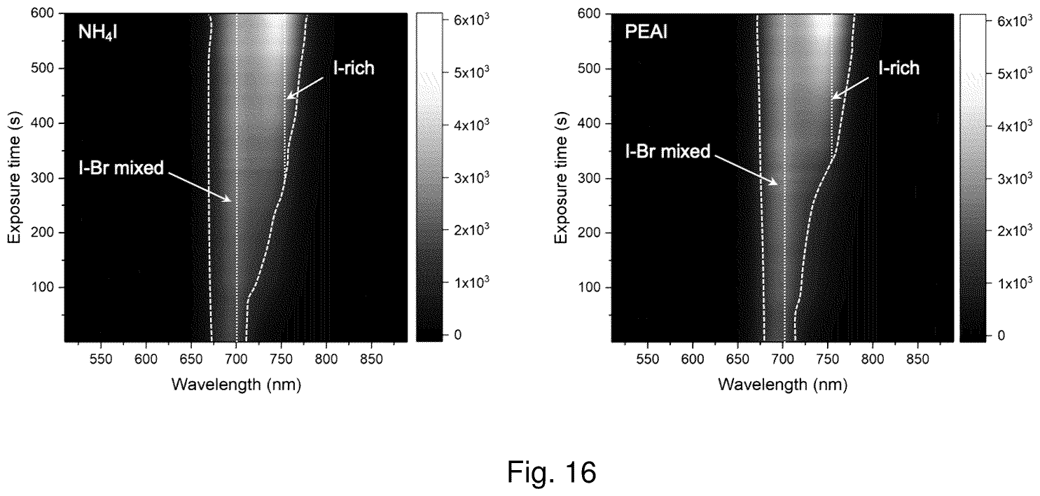

As for segregated I/Br mixed perovskites, the photogenerated charge carriers would funnel into I-rich regions due to their lower bandgaps than the surrounding non-segregated domains. In this case, a new PL peak with lower photon energy (˜1.64 eV) was observed in the unstable control perovskite. To intuitively study the extent of halide segregation, residual spectra deduced by subtracting the initial PL from the final spectrum was used, as shown in FIG. 15. The AQS-based perovskites exhibited only a small and symmetric valley-peak shape in their residual PL spectra, while a distinctly larger and asymmetric peak-valley shape was shown in that of the control sample. These significant changes in peak position and shape imply severe halide segregation. In addition, to exclude the effect of cationic substitutions on phase stability, the tdPL measurement for the perovskite films was carried out with NH₄I or PEAI as an additive, which exhibits negligible effect on alleviating phase segregation (FIG. 16).

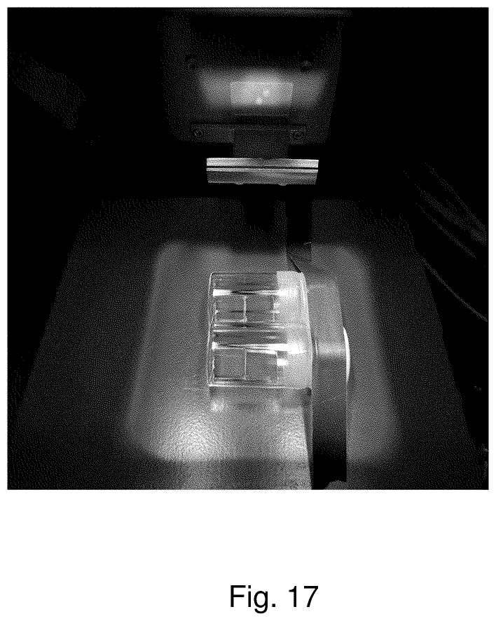

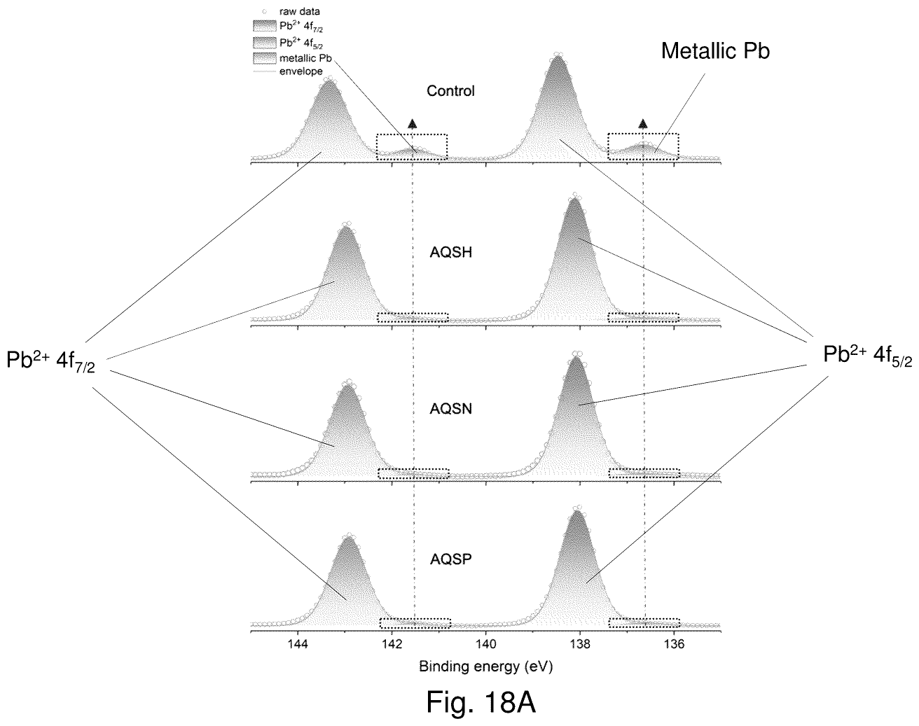

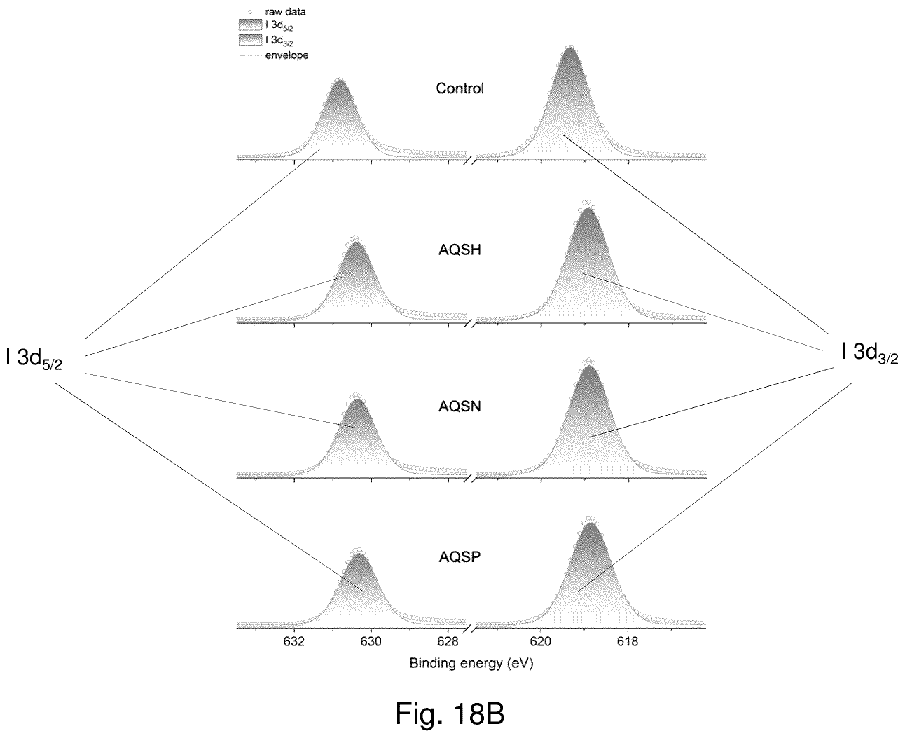

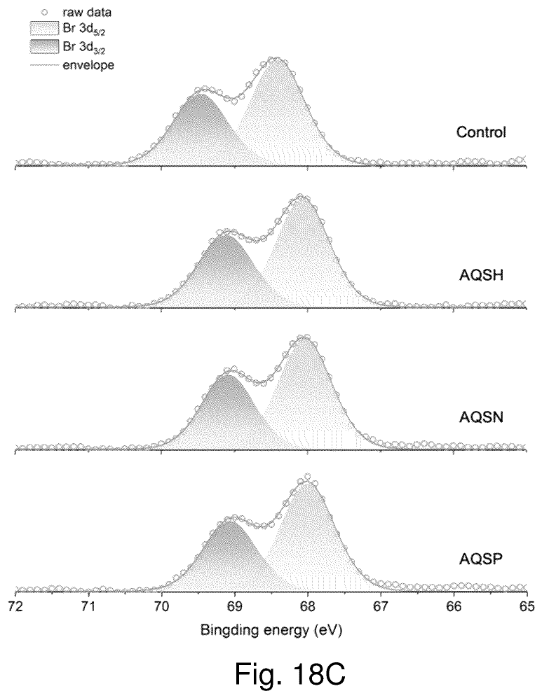

High-resolution X-ray photoelectron spectroscopy (XPS) was then used to study the surface property of the perovskite films that were aged under continuous illumination (AM 1.5G, 100 mW cm²) (FIG. 17). To verify whether the AQS-based redox mediators help suppress the formation of Pb⁰ and I⁰, focus was put on Pb, I, and Br elements (FIGS. 18A to 18C). The binding energies (BEs) at approximately 138.1 eV and 142.9 eV were assigned to be Pb 4f_{7/2} and Pb 4f_{5/2} of the AQS-based perovskite films, respectively. Comparatively, the BE of Pb 4f_{7/2} and Pb 4f_{5/2} slightly shifted to 138.5 eV and 143.3 eV for the control sample. Furthermore, two distinct shoulder peaks (i.e., 136.7 eV and 141.6 eV) originating from metallic Pb⁰ were observed in control perovskite, the primary decomposition product of perovskite under illumination.

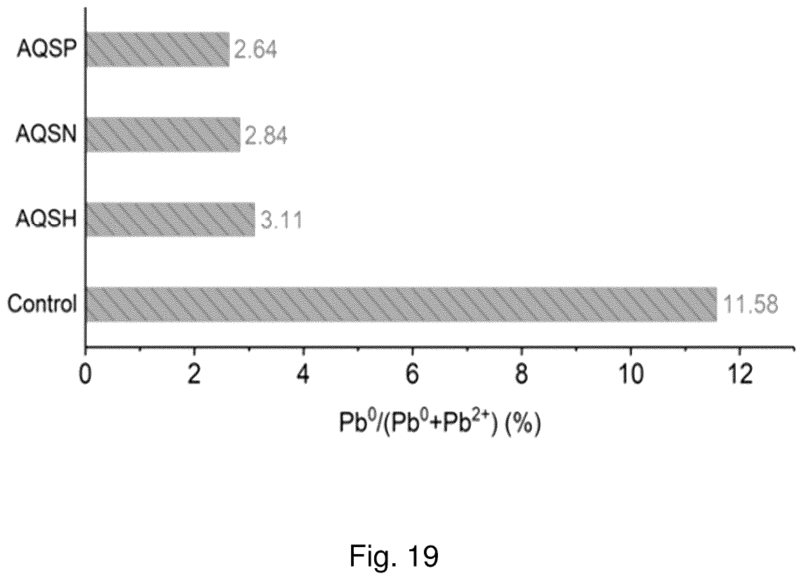

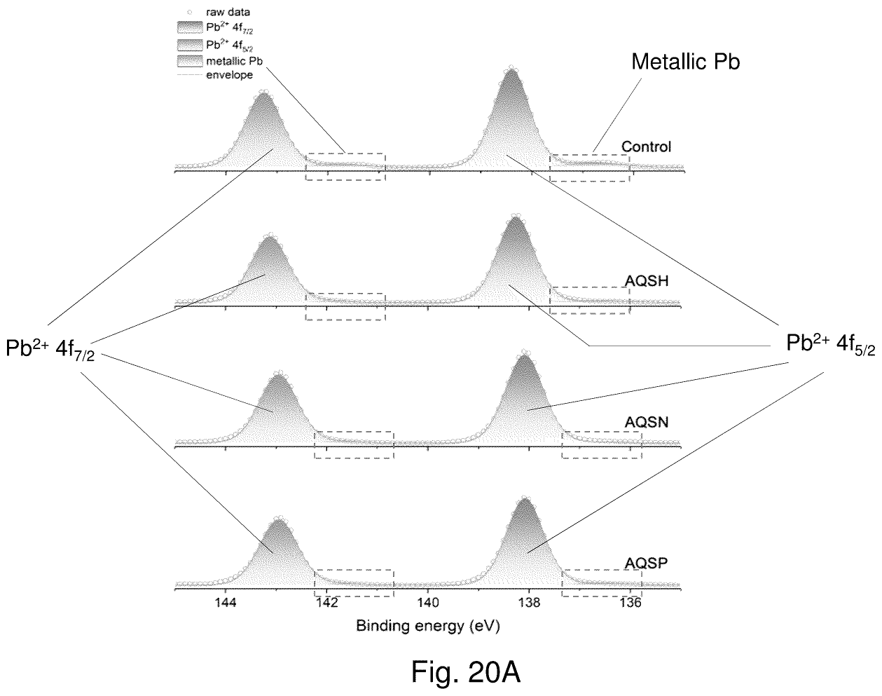

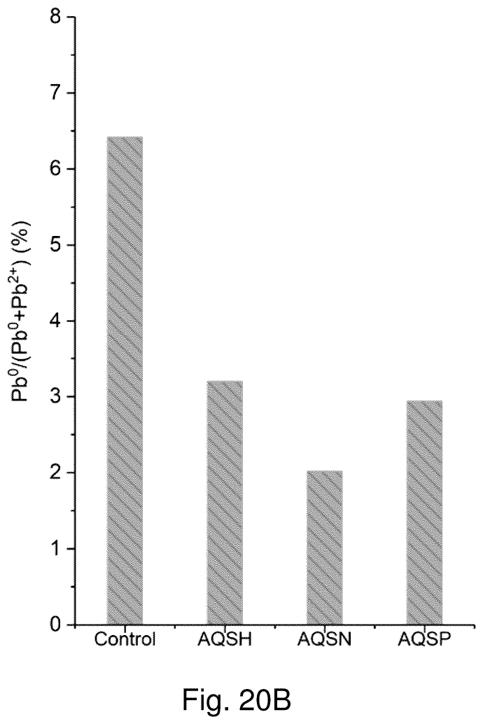

The atomic ratio of metallic Pb⁰ in total Pb was quantified and the results are shown in FIGS. 19, 20A and 20B. The control sample showed a high ratio of metallic Pb⁰ at 11.58%, whereas it was between 2.64-3.11% for AQS-based perovskites. Such a considerable reduction in Pb⁰ may be ascribed to its oxidation to Pb²⁺ mediated by AQS derivatives, as evident in the previous results shown in FIGS. 8 to 12.

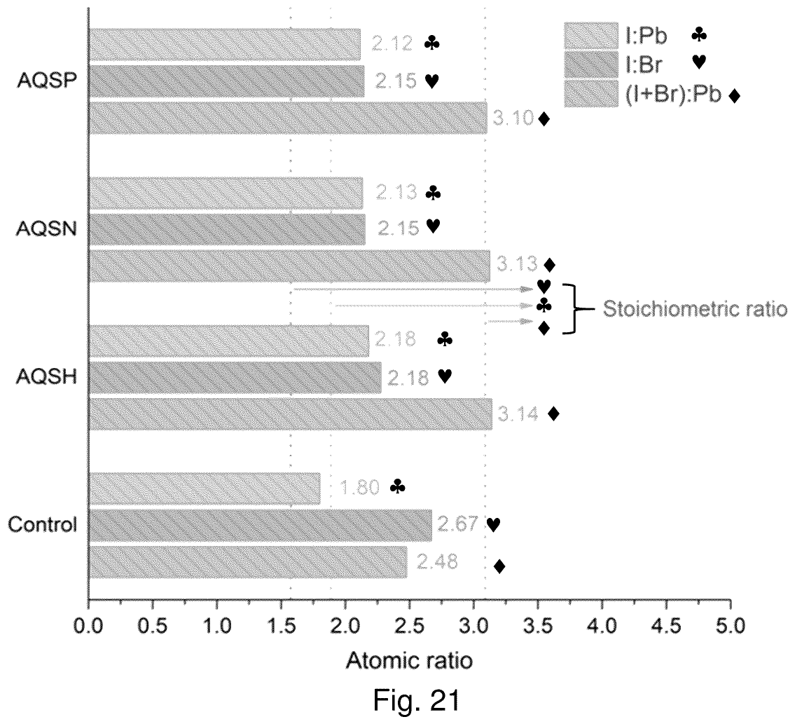

Regarding halide elements, two peaks were observed in the control perovskite, with BEs of 619.3 eV and 630.8 eV, originating from I 3d₅s₂ and I 3d₃/2, respectively (FIG. 18B). Given the volatile nature of iodine products, the relative atomic ratios were used to study the halide distribution on the illuminated perovskite surface (FIG. 21). It is noted that I/Pb atomic ratios of AQS-based perovskite films (2.12-2.18) were slightly higher than the stoichiometric ratio (denoted by the dashed lines), while the (I+Br)/Pb atomic ratios close to stoichiometric ratio (3.09). This non-uniformity in composition might be ascribed to different nucleation/crystallization dynamics for I- and Br-based perovskites. Nevertheless, it is found that the loss of halide species in control perovskite, as the atomic ratio of (I+Br)/Pb (2.48) was far below its stoichiometric ratio of 3.09. Considering its I/Pb atomic ratio of 1.80, this halide content loss is attributed to be the iodine species because of their lower oxidation potential than bromides (1.065 V). Meanwhile, the increased I/Br ratio of 2.67 in the control sample suggests the faster migration of Br along the direction of incident light as reported.

### Example 6

### Structural Properties and Density Functional Theory (DFT) Calculations

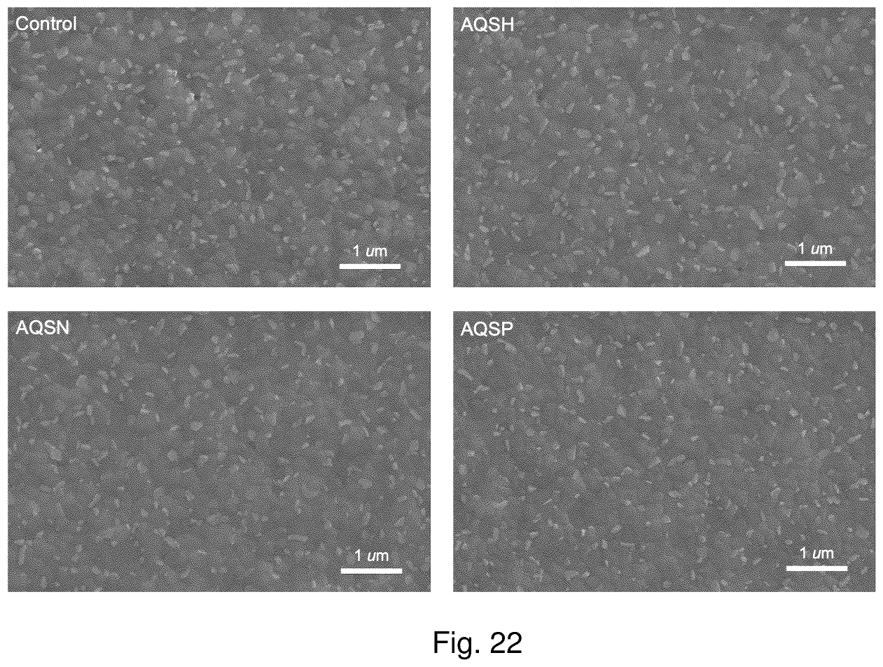

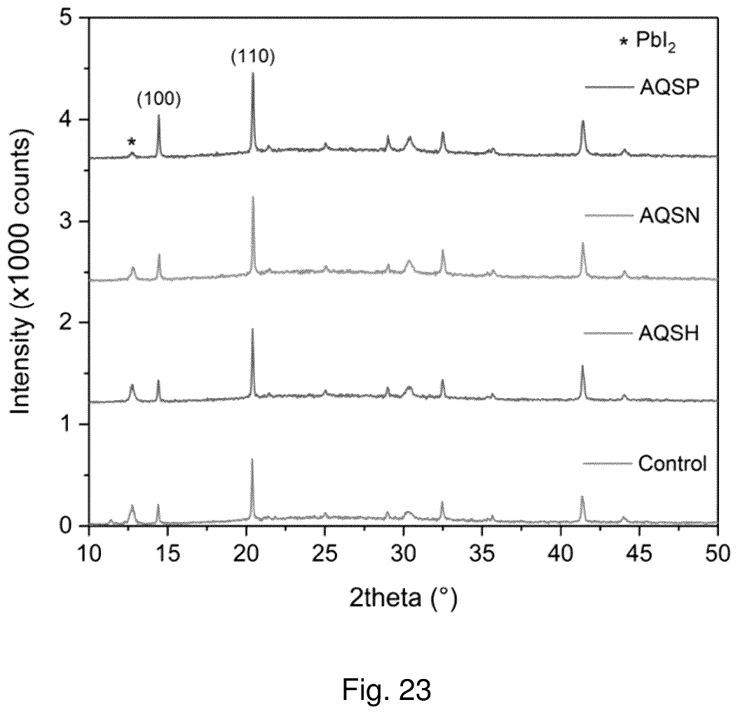

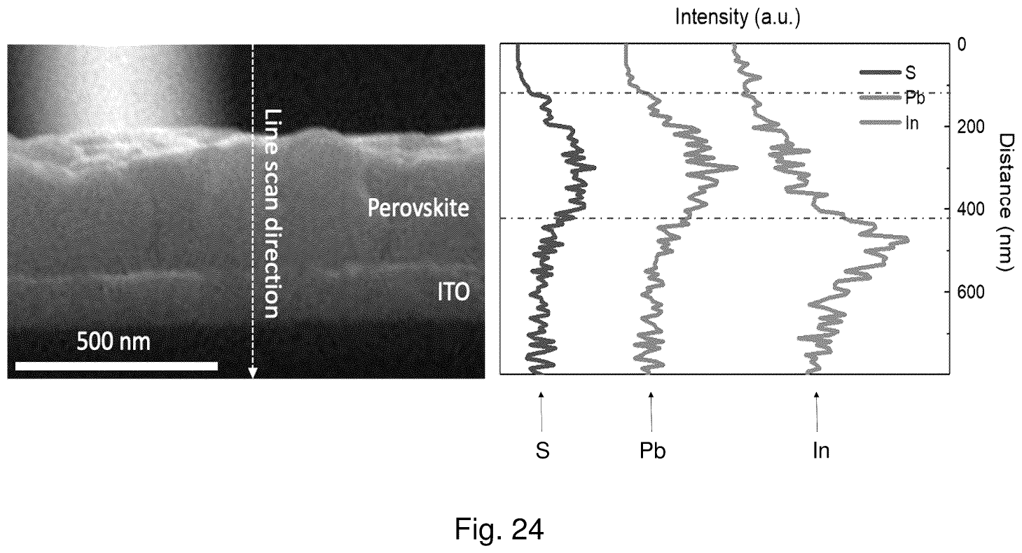

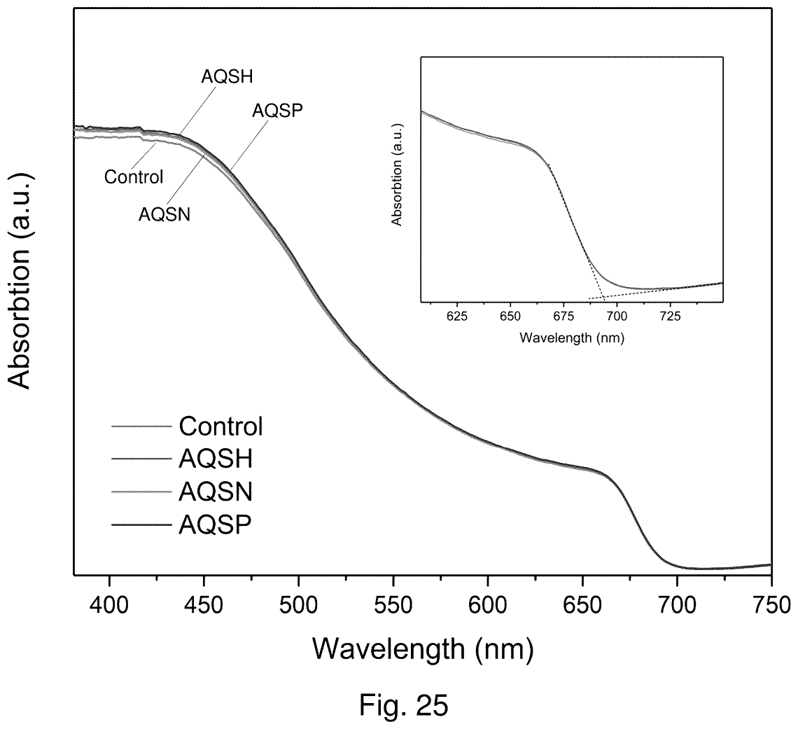

The structural properties of the perovskite films were studied by using scanning electron microscopy (SEM) and XRD, and the results are shown in FIGS. 22 and 23. All XRD patterns revealed two prominent diffraction peaks at 20 of 14.4° and 20.4°, corresponding to the (100) and (110) lattice planes of perovskites. Lower intensity of the diffraction peak from PbI₂ was observed for the AQSN- and AQSP-based perovskites, which might be ascribed to the modulation effect of the substituted cations on perovskite growth. The top-viewing SEM images exhibited no obvious differences in the morphology of the samples. The distribution of AQS-derivatives within the perovskite layer was profiled by cross-sectional SEM-energy dispersive spectroscopy (SEM-EDX), which shows uniform distribution (FIG. 24). It is also determined the optical bandgap of the perovskite films to be approximately 1.79 eV by their UV-vis absorption spectra, as presented in FIG. 25.

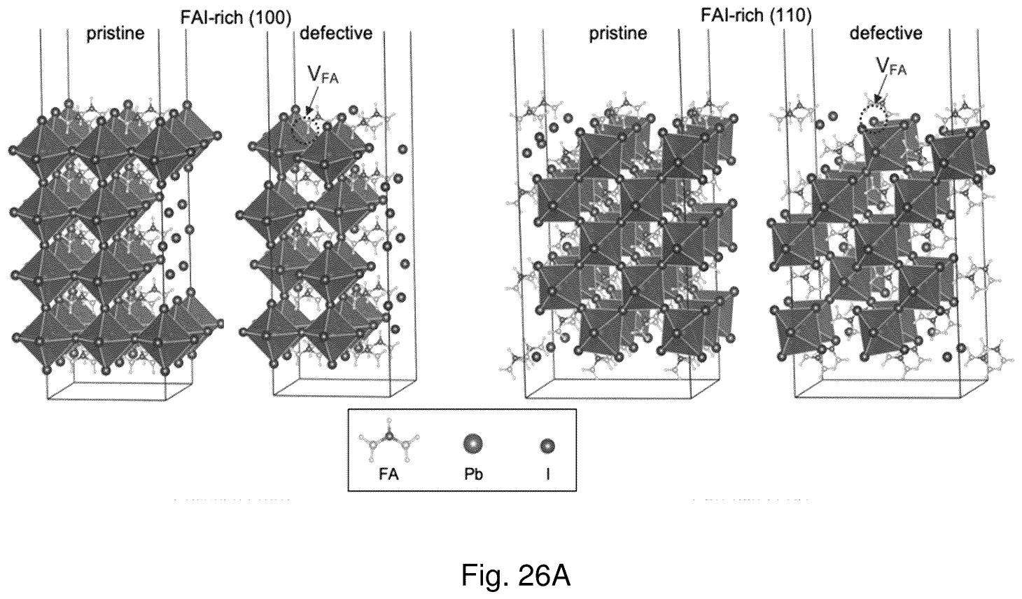

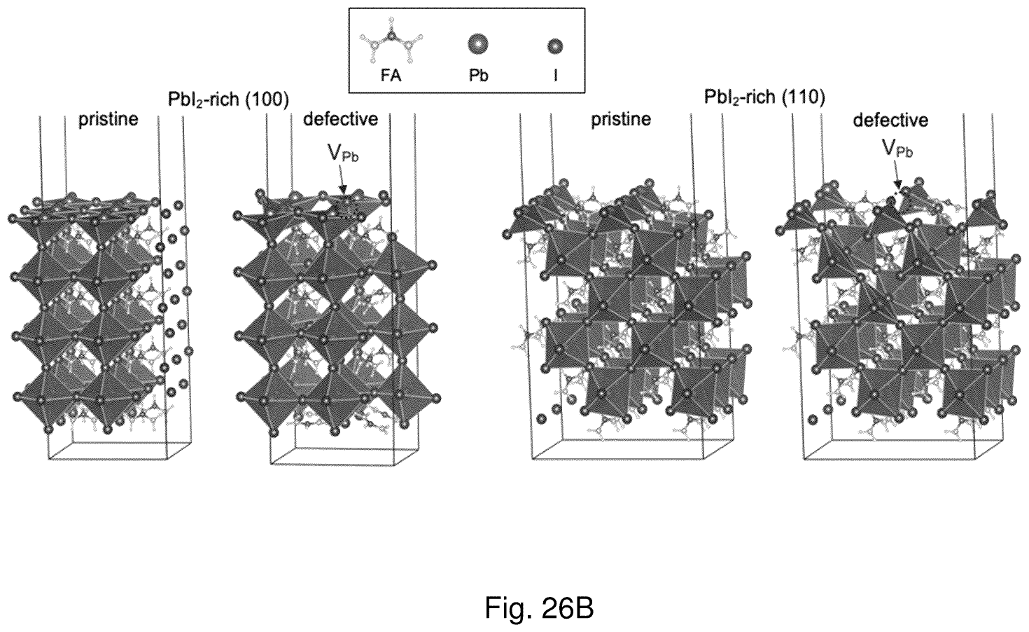

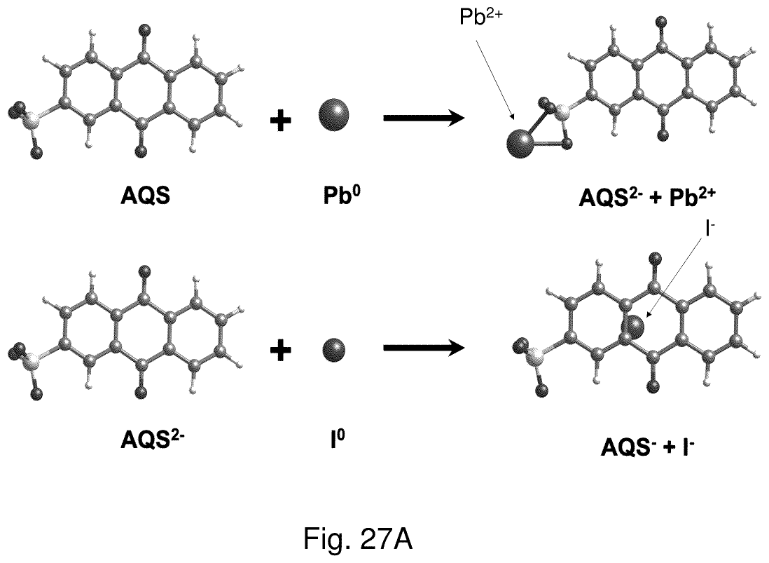

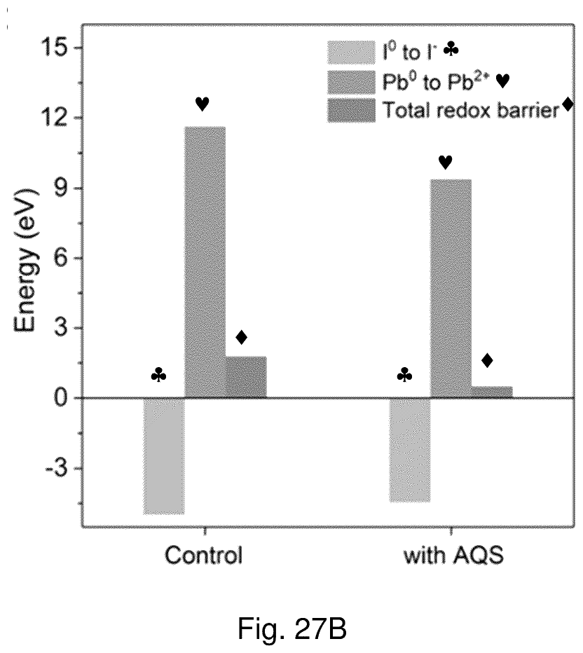

Based on the above structural analysis, density functional theory (DFT) calculations was performed on redox mediator/perovskite interfacial models to gain insights into the role of AQS derivatives in facilitating the overall redox reaction and passivating perovskite, as well as to estimate their interactions. Two representative perovskite surfaces were taken into consideration, each with different terminations (FAI- and PbI₂-rich) and surface defects (V_{FA} and V_{Pb}), as depicted in FIGS. 26A and 26B. The energy barriers of the half-reactions associated with the elimination of Pb⁰ and I⁰ were first calculated (FIGS. 27A and 27B). The results indicated that the half-reaction from Pb⁰ to Pb²⁺ presented a considerable energy barrier (11.6 eV), while the reduction from I⁰ to I⁻ was more thermodynamically favored for pristine perovskite (-5.0 eV). Upon introducing AQS derivatives onto perovskite surface, the energy barriers for both half-reactions were reduced. The total energy barrier for the redox reaction was lowered by more than threefold (from 1.76 eV to 0.49 eV), verifying the ability of these molecules in facilitating electron shuttling within the perovskite.

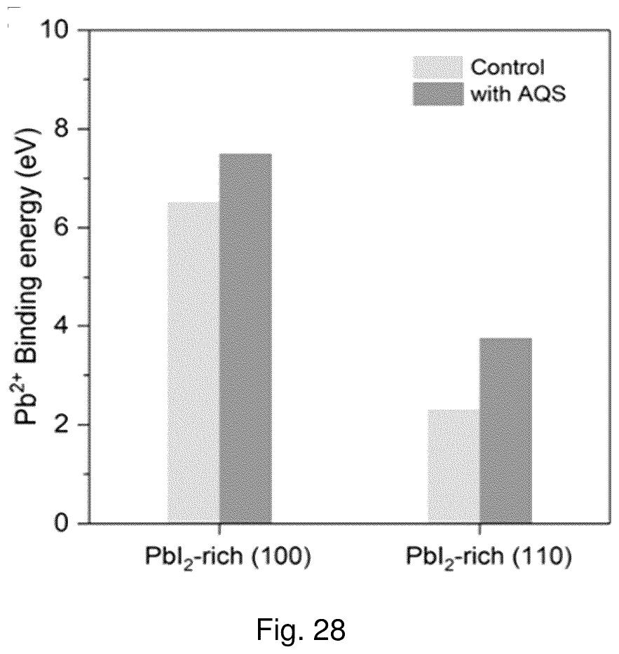

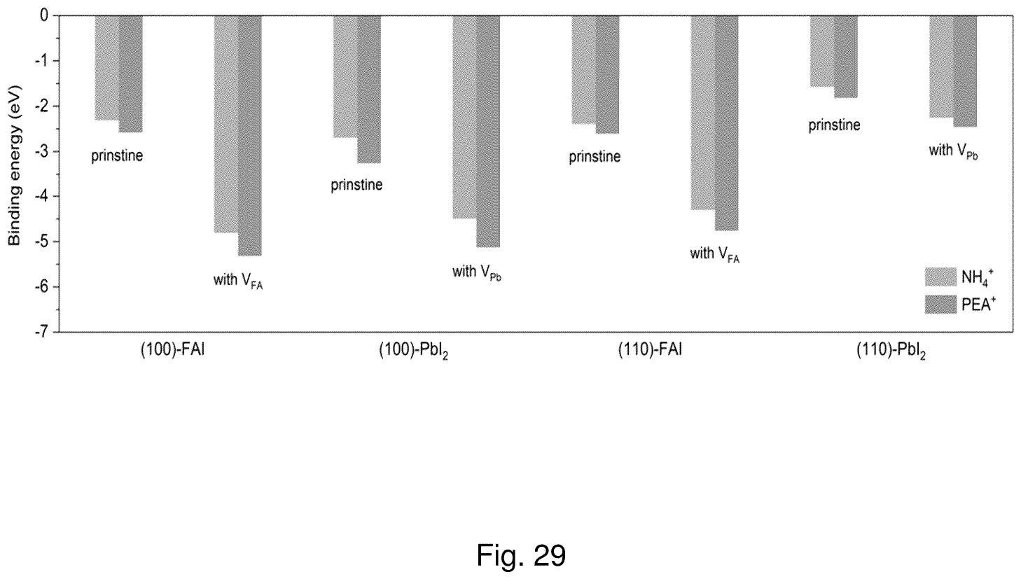

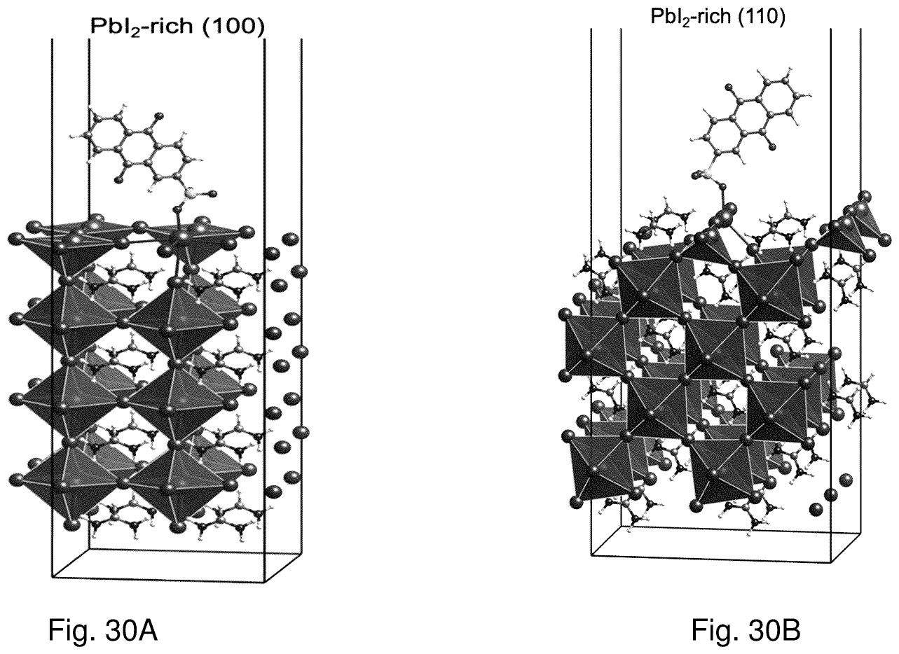

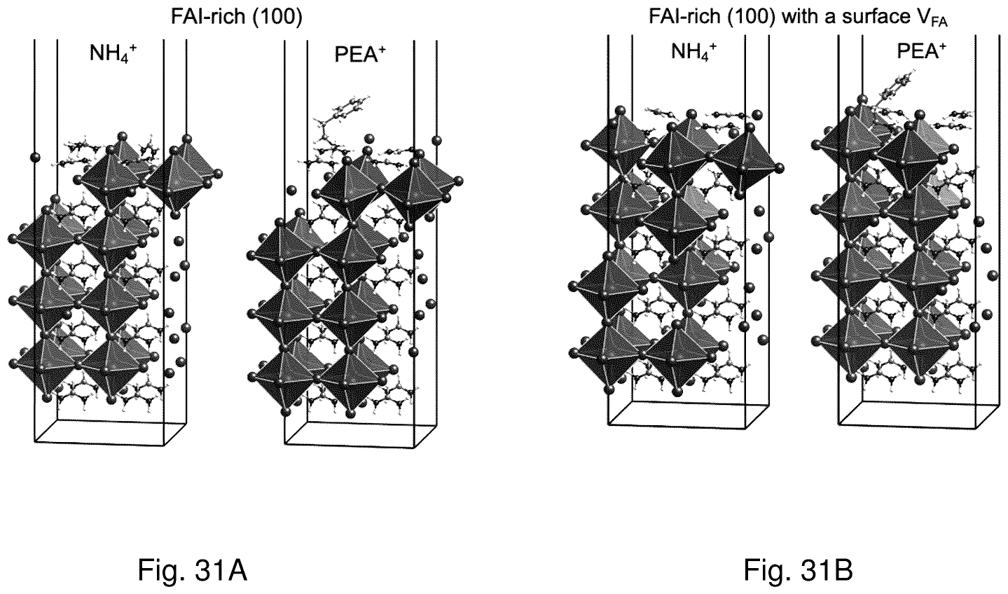

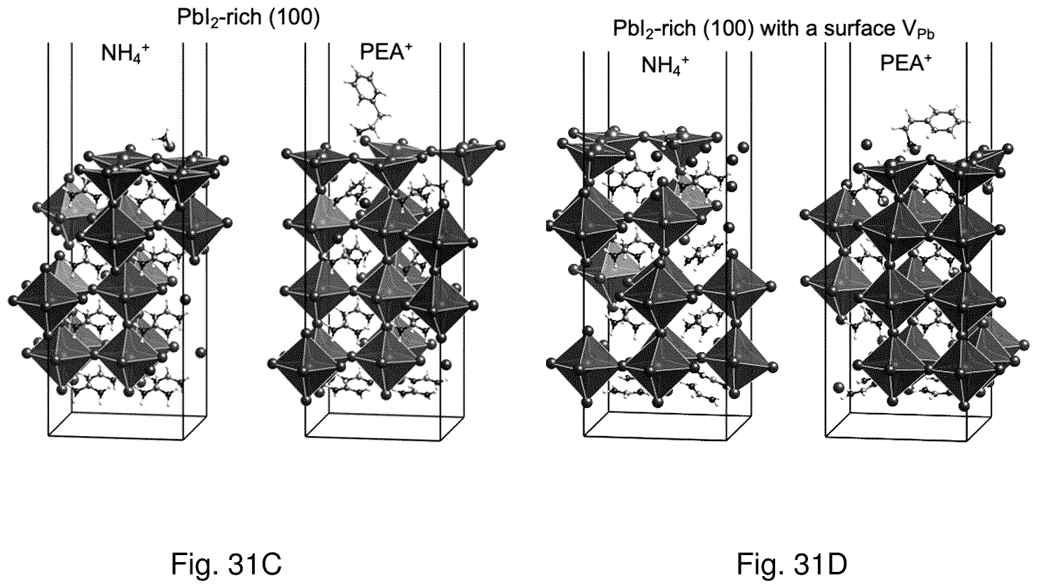

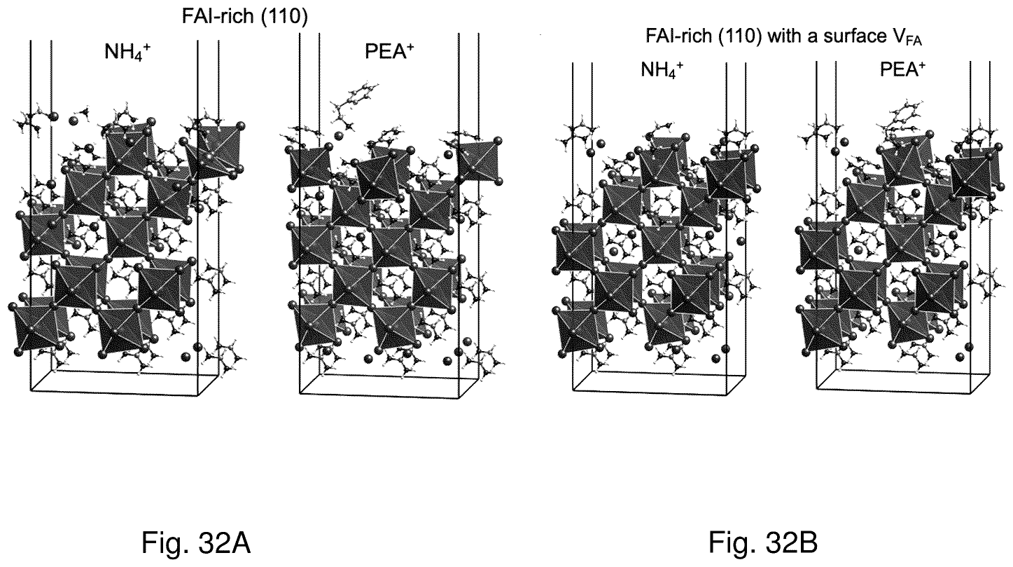

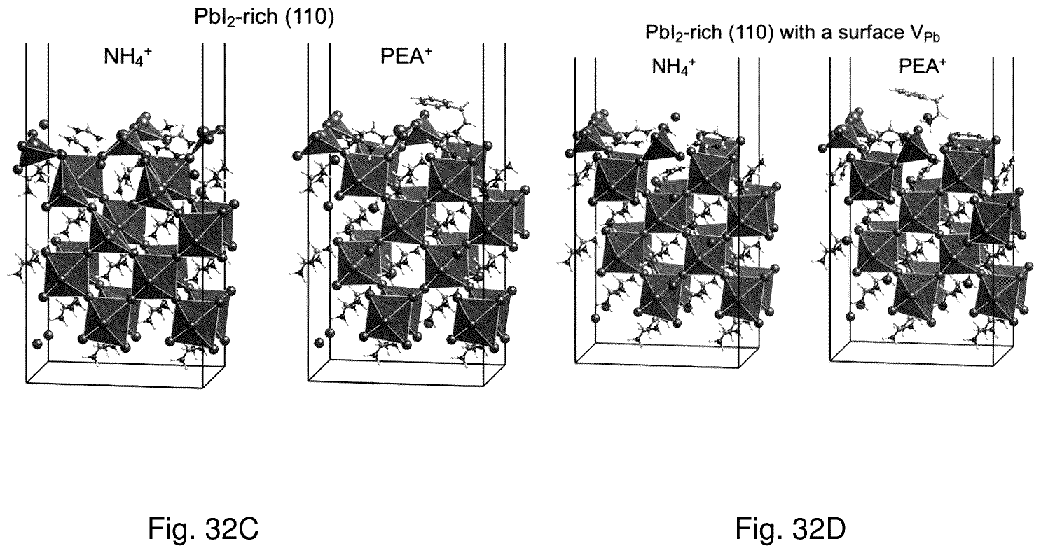

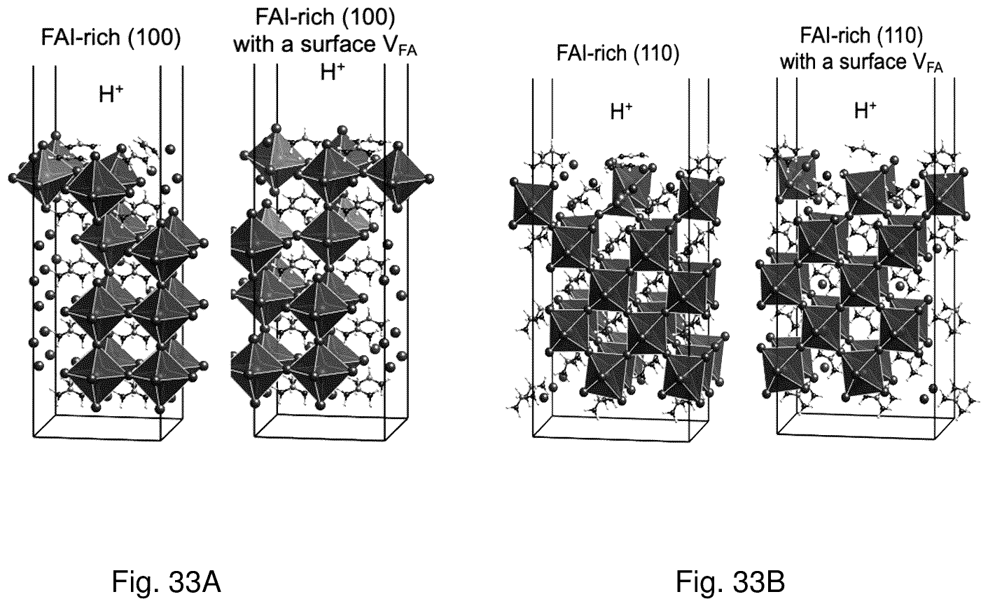

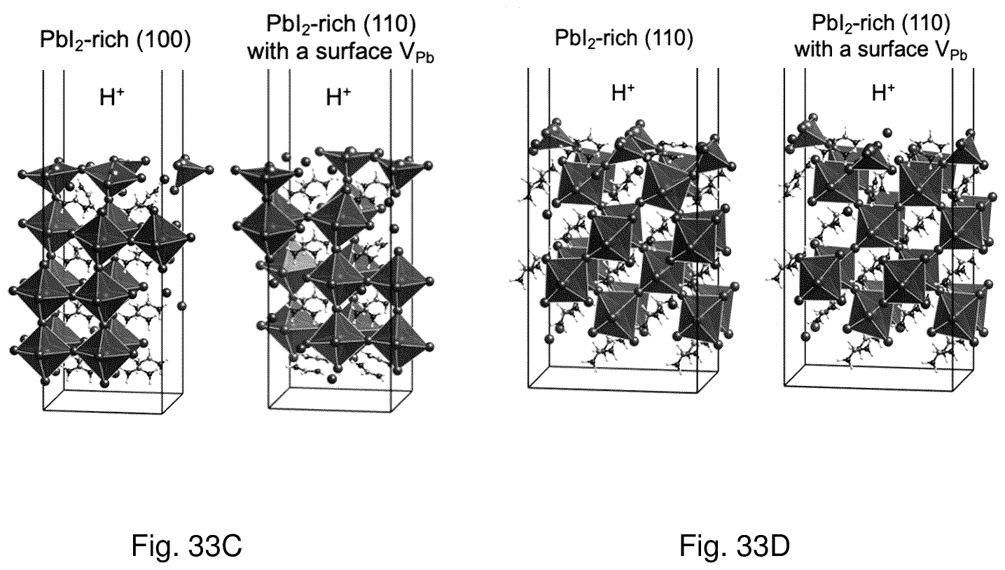

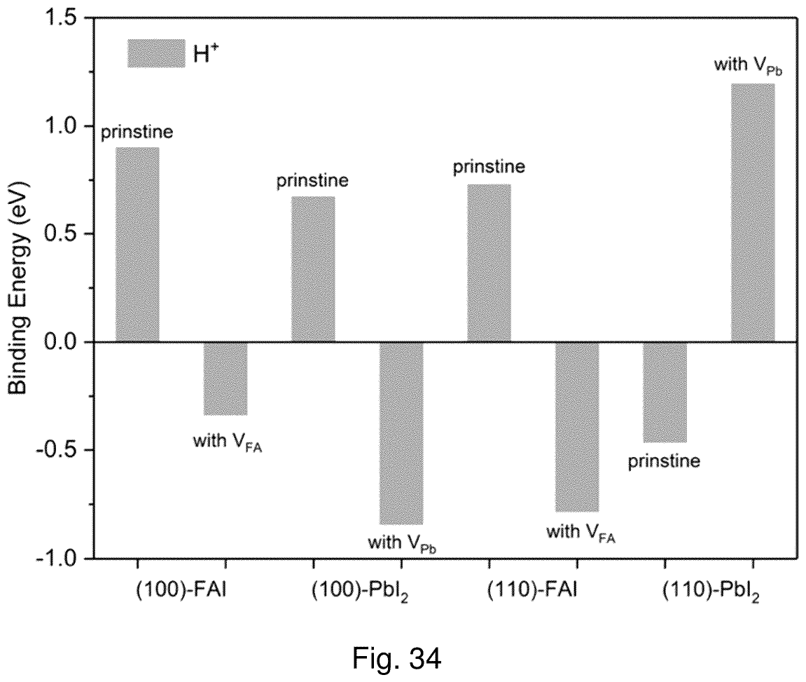

As discussed in Example 4, the AQS core governs the redox capability, while the substitution of —H by —NH₄ and —PEA affords the molecules with an additional defect passivation effect. The binding energies between AQS— and Pb²⁺ were calculated to be 7.50 eV and 3.75 eV at the PbI₂-rich (100) and (110) surfaces, respectively, which were higher than those of the control perovskites (FIG. 28). The stronger binding with Pb²z is crucial for stabilizing the perovskites. Moreover, the binding energy between NH₄⁺/PEA⁺ and perovskite surfaces were calculated (FIG. 29). The optimized crystal structures of the redox mediators-modified perovskites are presented in FIGS. 30A-30B, 31A-31D, 32A-32D, 33A-33D, and 34. The results suggest that the PEA⁺ cation provides a stronger defect passivation effect than NH₄⁺, regardless of lattice plane and surface properties, as it consistently exhibits higher binding energies to the perovskite surface.

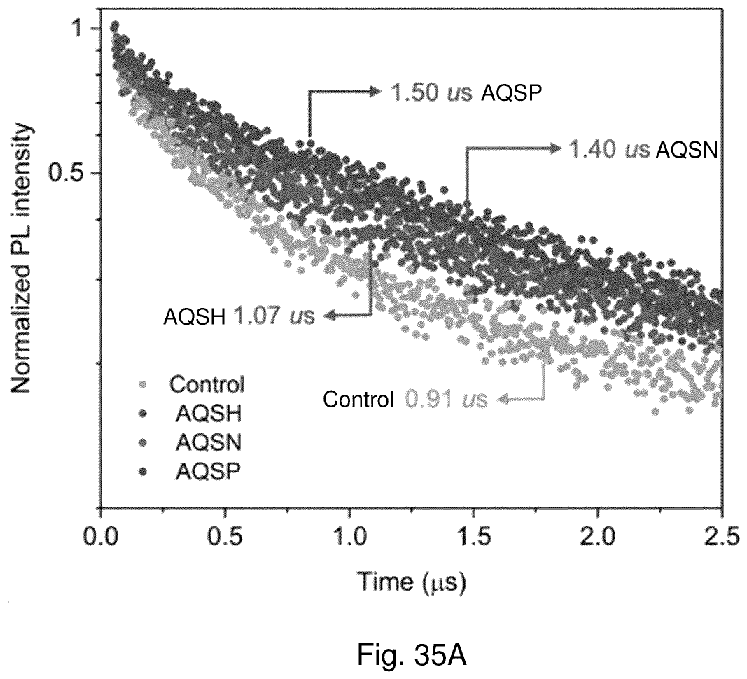

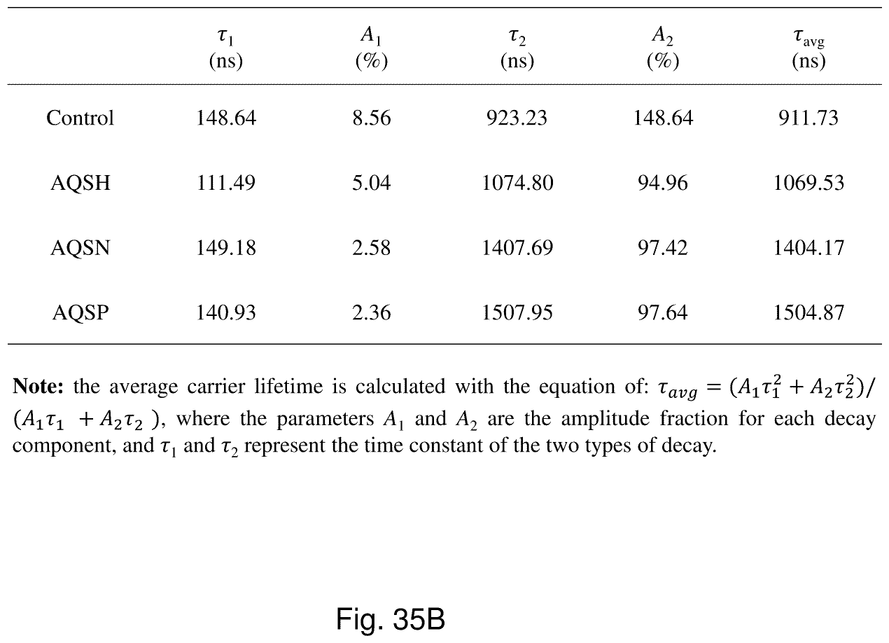

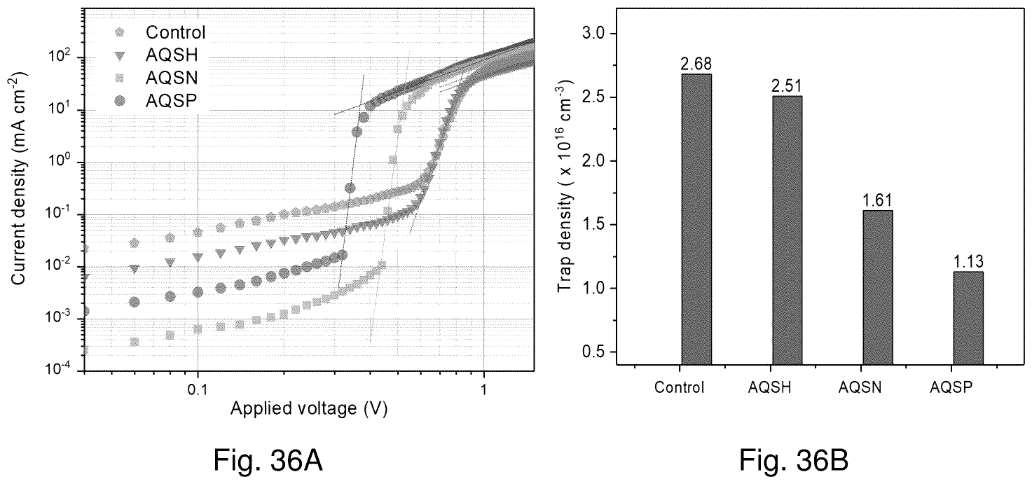

Time-resolved PL (trPL) measurements were conducted on the perovskite films to investigate the defect passivation effect of AQS derivatives (FIG. 35A). The average carrier lifetime (τ_{avg}) was calculated to be 0.91 μs for control perovskite, and slightly increased to 1.07 μs for AQSH-based perovskite (FIG. 35B). Notably, the carrier lifetime of 1.40 μs and 1.50 μs were obtained for AQSN- and AQSP-based perovskite, respectively. As the prolonged carrier lifetime usually indicates enhanced radiative recombination, space-charge-limited current (SCLC) method was used to quantify the trap density (Nₜᵣₐₚ) in the perovskites (FIG. 36A). The trap-filled limit voltages (V_{TFL}) were first extracted by fitting the J-V curves, then the Nₜᵣₐₚ values were calculated to be 2.68×10¹⁶, 2.51×10¹⁶, 1.61×10¹⁶, and 1.13×10¹⁶ cm⁻³ for control, AQSH-, AQSN- and AQSP-based perovskites, respectively (FIG. 36B).

### Example 7

### Photovoltaic Performance of PSCs and PO-TSCs

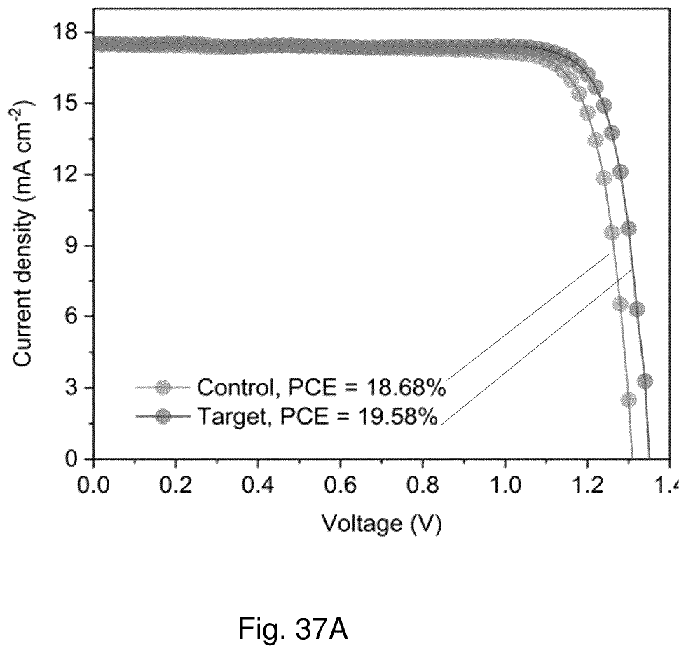

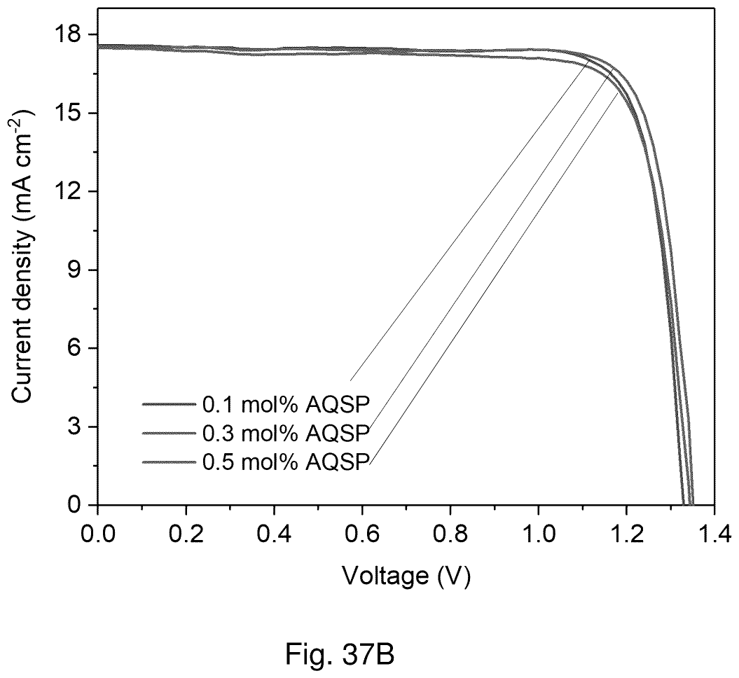

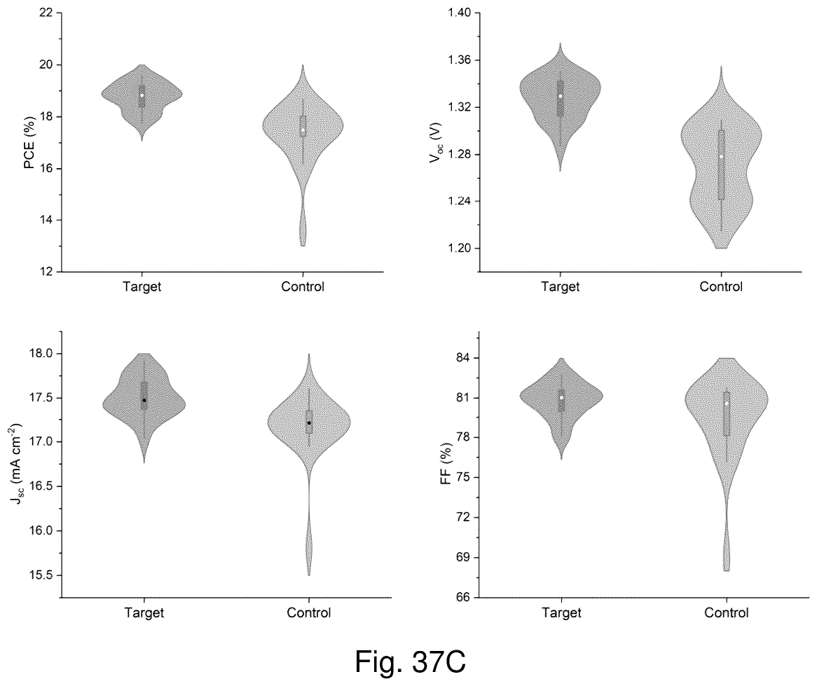

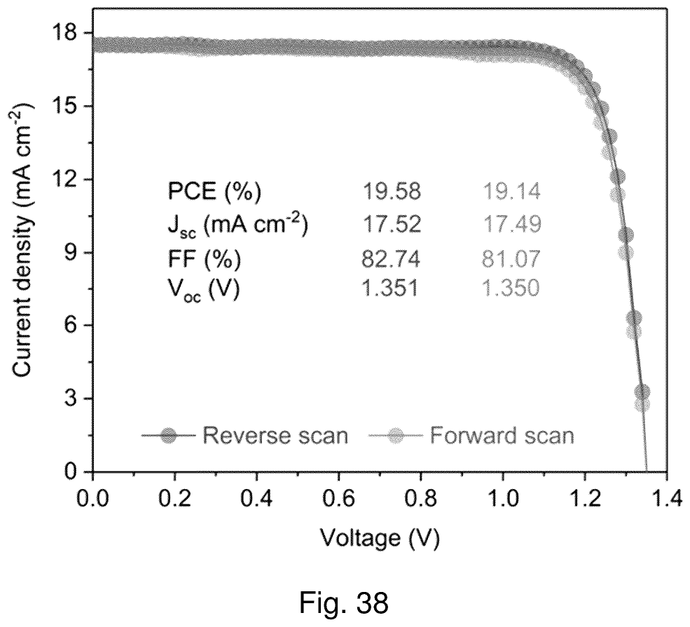

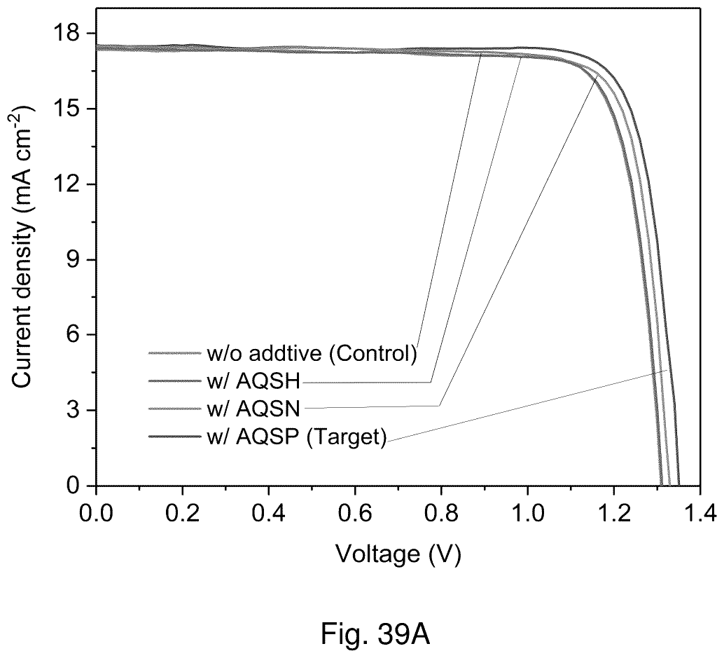

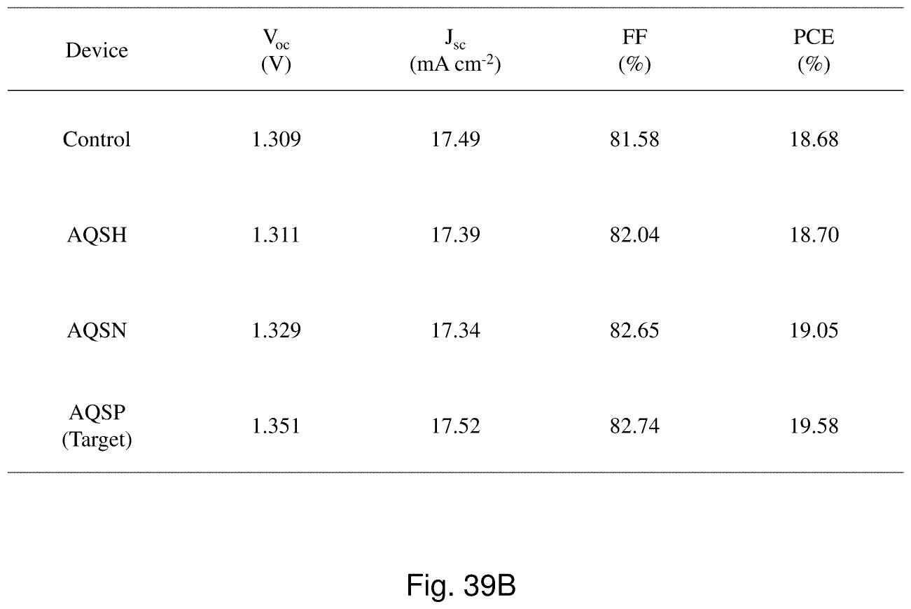

To further investigate the suppressed phase segregation and non-radiative recombination properties imparted by AQSP additive, single-junction wide-E_{g} PSCs were fabricated based on the structure of glass/ITO/DC-PA/perovskite/C60/bathocuproine (BCP)/Ag, where ITO is indium tin oxide, and DC-PA denotes a p-type self-assembled monolayer (SAM). The characteristics of the devices based on the perovskite without and with AQSP (denoted as control and target, respectively) are discussed as follows. The control device exhibited a decent PCE of 18.68%, while the optimized target device achieved an efficiency of 19.58%, mainly due to the enhanced V_{oc} from 1.309 V to 1.351 V (FIGS. 37A to 37C). The target device also showed negligible hysteresis as the PCE of 19.14% could be obtained under forward scan direction (FIG. 38). The photovoltaic performance of the devices based on the perovskites with different AQS derivatives is provided in FIG. 39A and summarized in FIG. 39B.

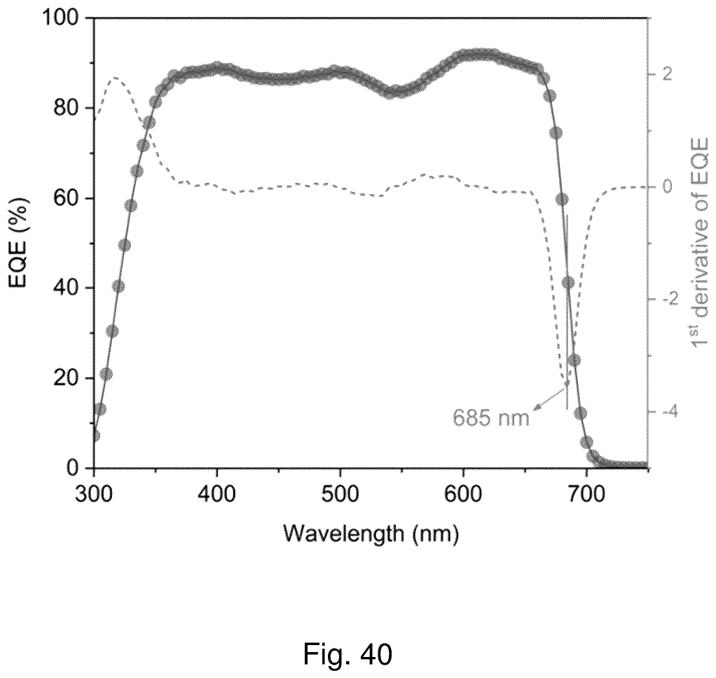

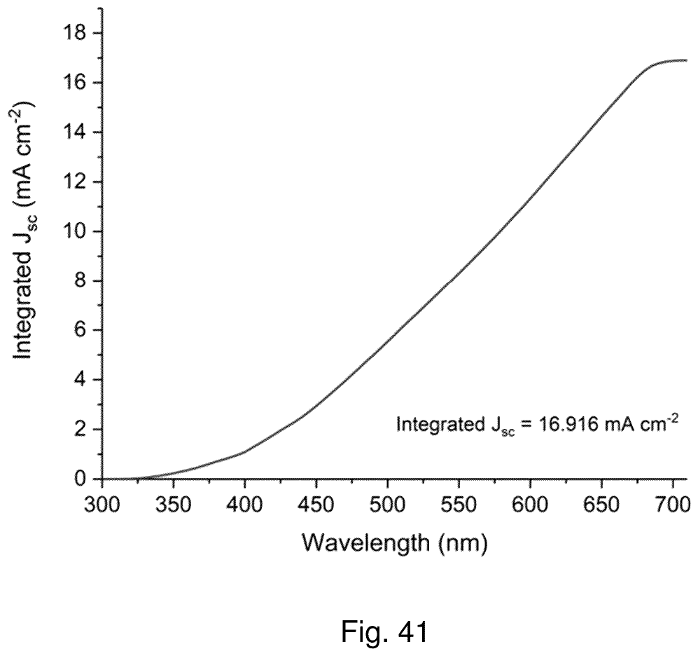

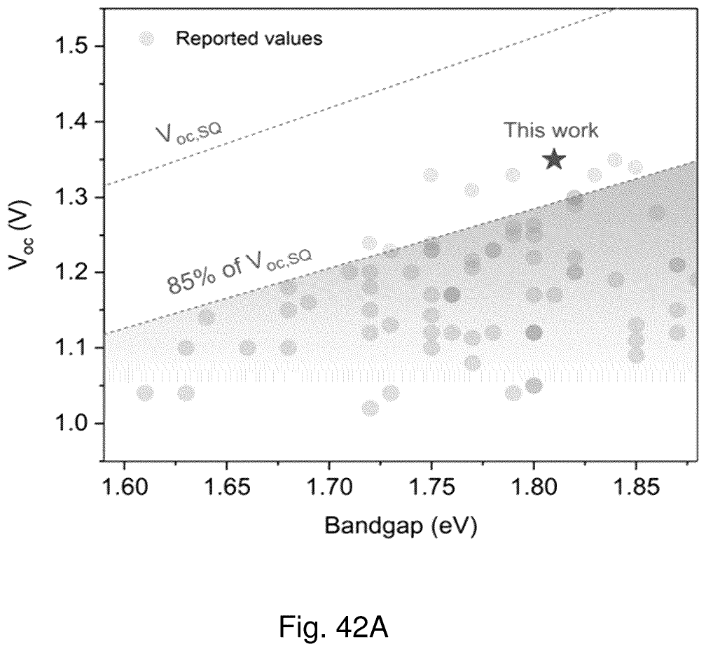

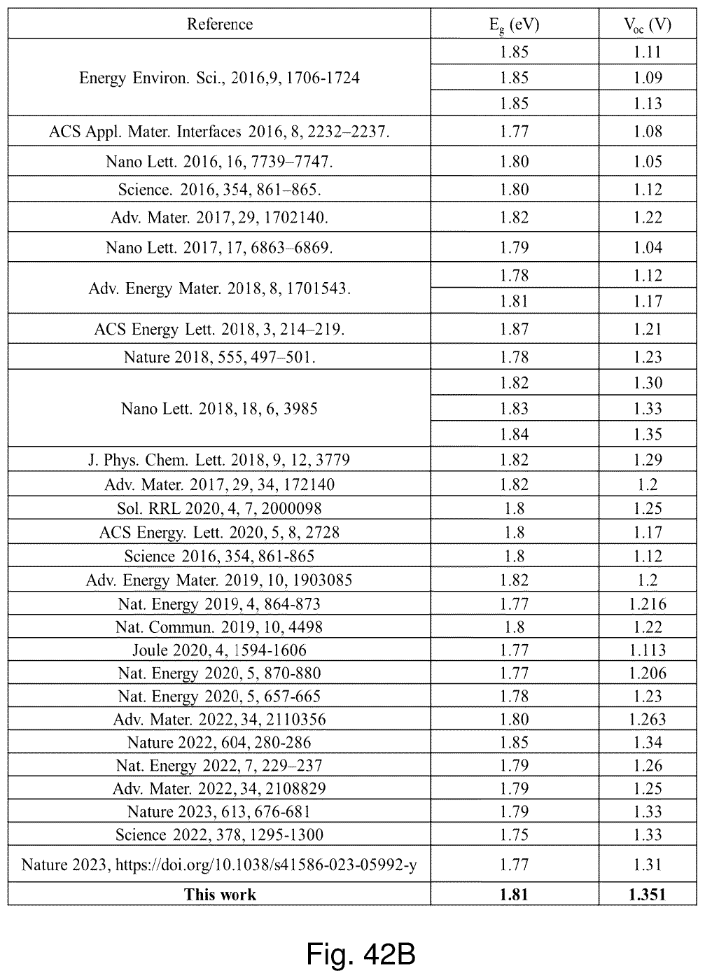

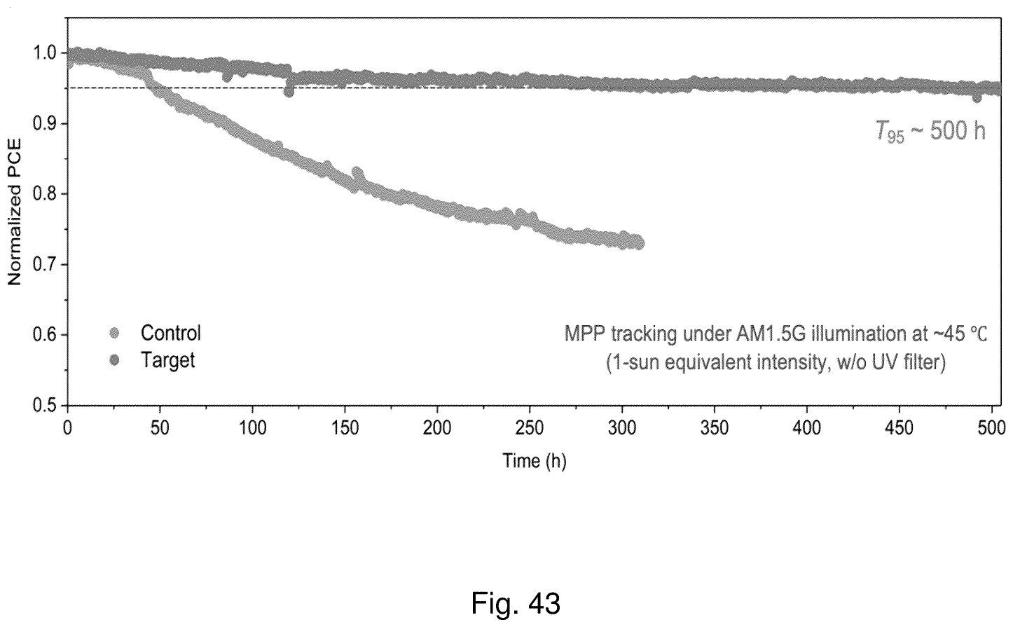

The bandgap of the target device was determined to be 1.81 eV by differentiating its external quantum efficiency (EQE) spectrum, which exhibited an inflection point at 685 nm (FIG. 40). The integrated short-circuit current density (J_{sc}) from the EQE matched well with the value extracted from the J-V curve (FIG. 41). The V_{oc} deficit (E_{g}−qV_{oc}) of the target device was calculated to be 0.459 V, which is the lowest value reported so far for PSCs with an E_{g} of ˜1.80 eV (FIGS. 42A and 42B). Furthermore, we achieved significantly improved long-term stability for the target cell (initial efficiency=18.98%), which retained 95% of its initial PCE after operating under 1-sun illumination at its maximum power point (MPP) for 500 hours (FIG. 43). In contrast, the efficiency of the control cell (initial efficiency=18.08%) degraded rapidly (>20%) within 300 hours of operation.

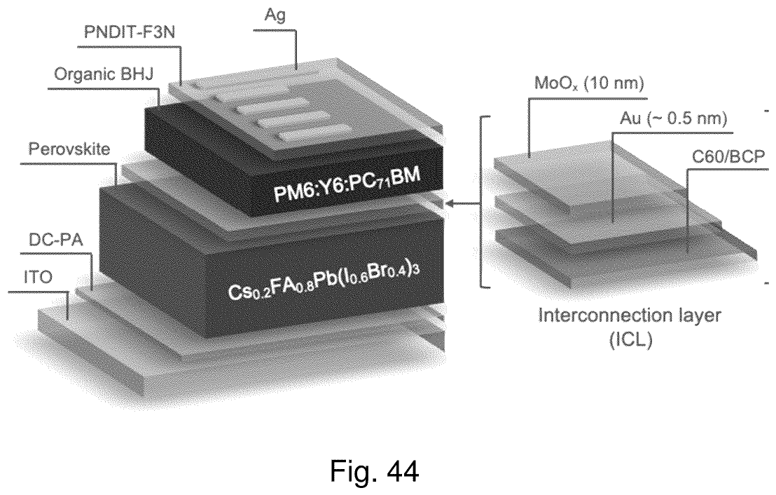

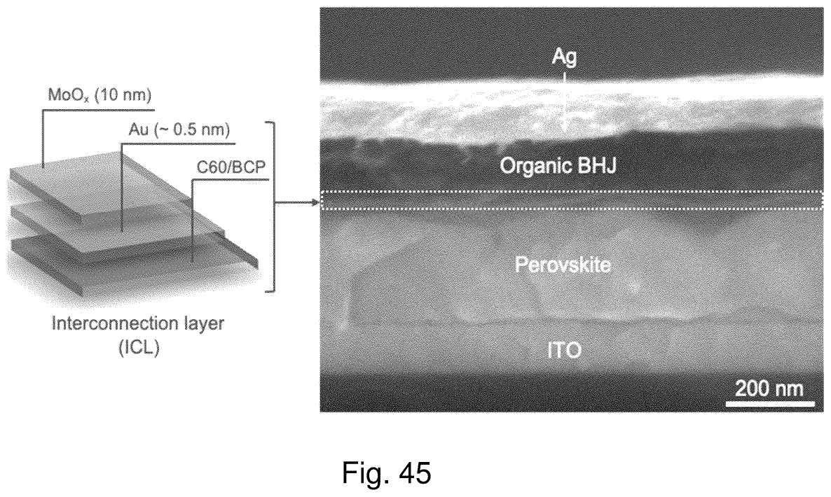

The above wide-E_{g} perovskite was integrated with organic semiconductors for constructing monolithic PO-TSCs. The PM6:Y6:PC₇₁BM ternary blend was used as the bulk heterojunction layer in the narrow-E_{g} rear subcell. The device structure of PO-TSC is depicted in FIG. 44, where BCP/Au/MoOₓ acts as the interconnecting layer (ICL). To obtain a good trade-off between transparency and electrical properties, a 0.5 nm-thick Au was utilized as the recombination layer. The thickness of the perovskite and organic BHJ layer was optimized to be ˜260 nm and ˜150 nm, respectively, to ensure efficient photon utilization and current matching between the two subcells (FIG. 45).

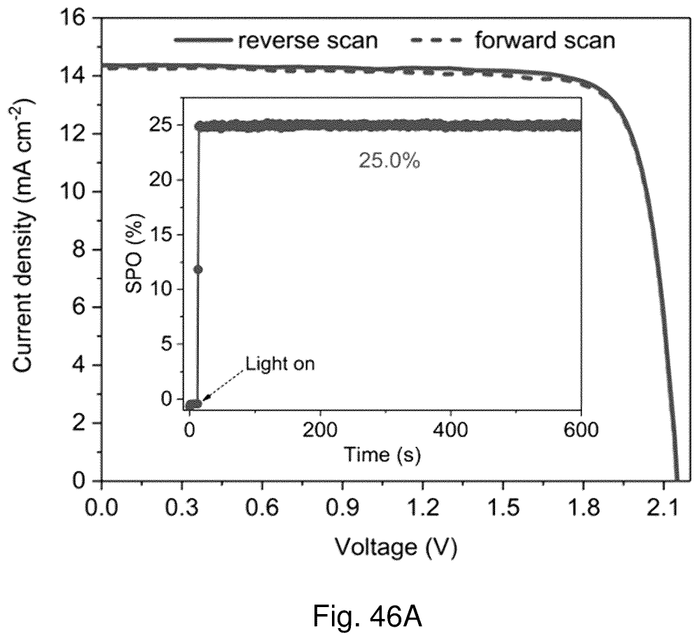

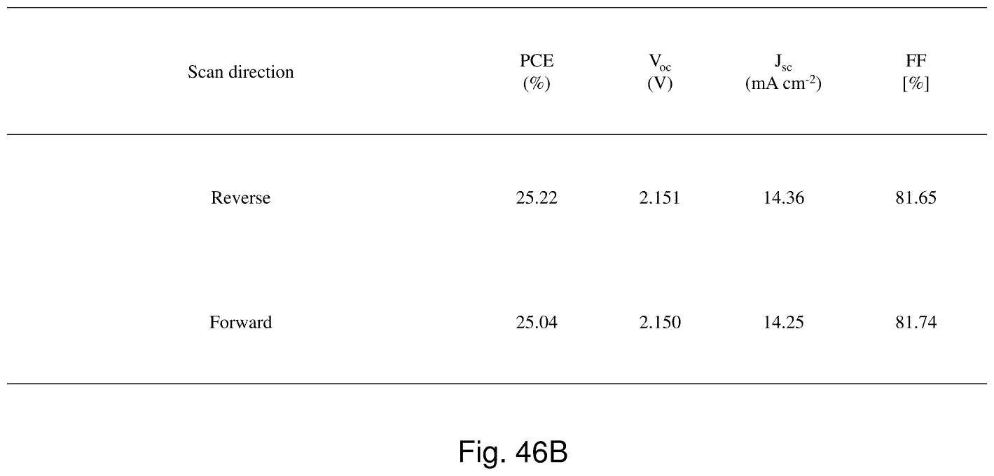

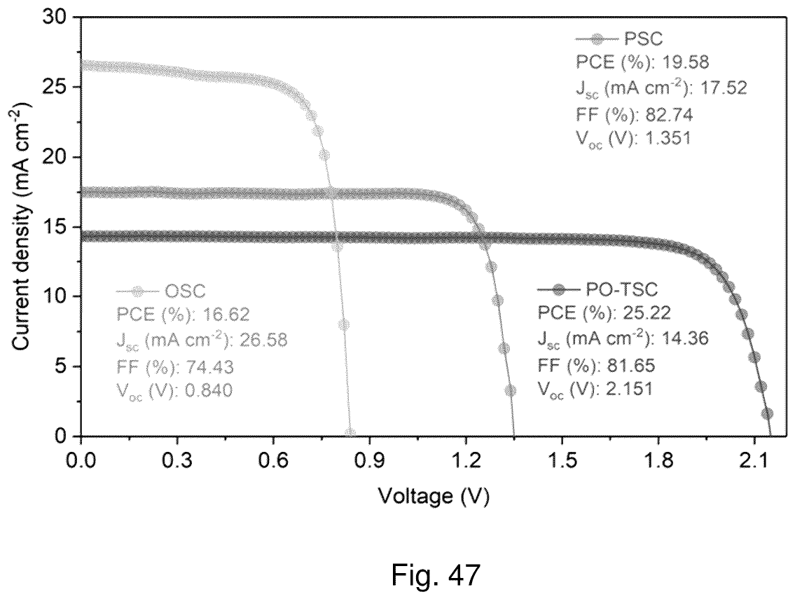

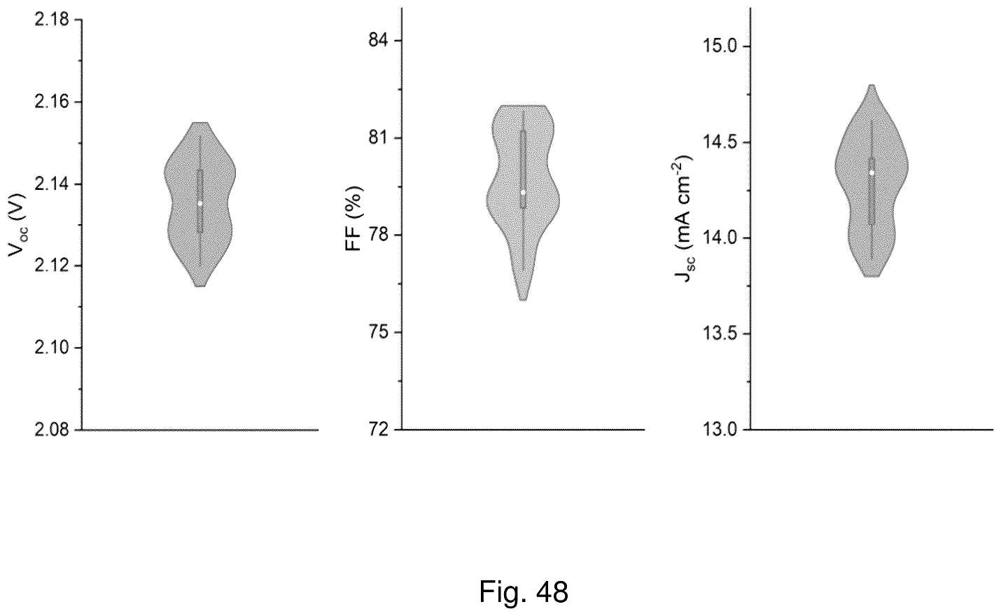

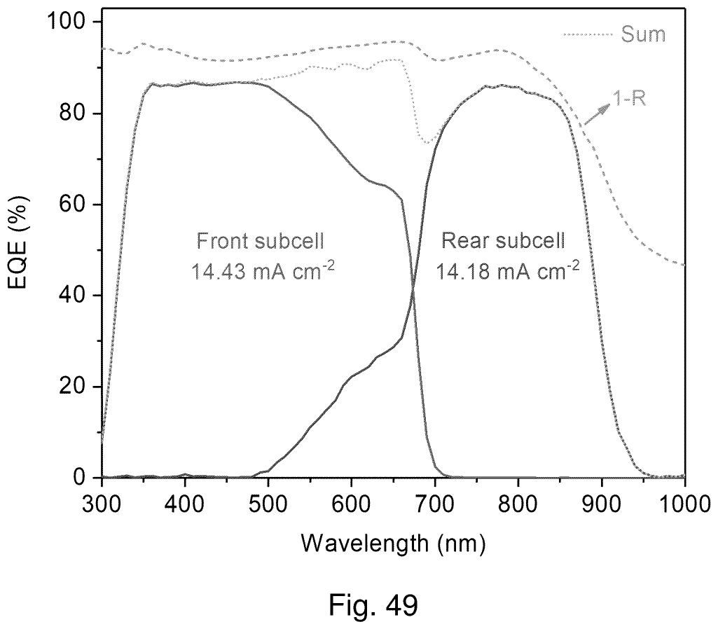

Remarkably, the tandem solar cell achieved an impressive PCE of 25.22% with a high V_{oc} of 2.151 V, J_{sc} of 14.36 mA cm², and fill factor (FF) of 81.65%, exhibiting negligible hysteresis and a stabilized power output (SPO) of 25.00%. (FIGS. 46A and 46B). Such high efficiency can be ascribed to the low voltage loss from the perovskite subcell and ICL (FIGS. 47 and 48). The tandem cell of this work also showed good current matching, where the J_{sc} of 14.84 mA cm⁻³² and 14.38 mA cm⁻³² are integrated from the EQE spectrum of perovskite and organic subcells, respectively (FIG. 49).

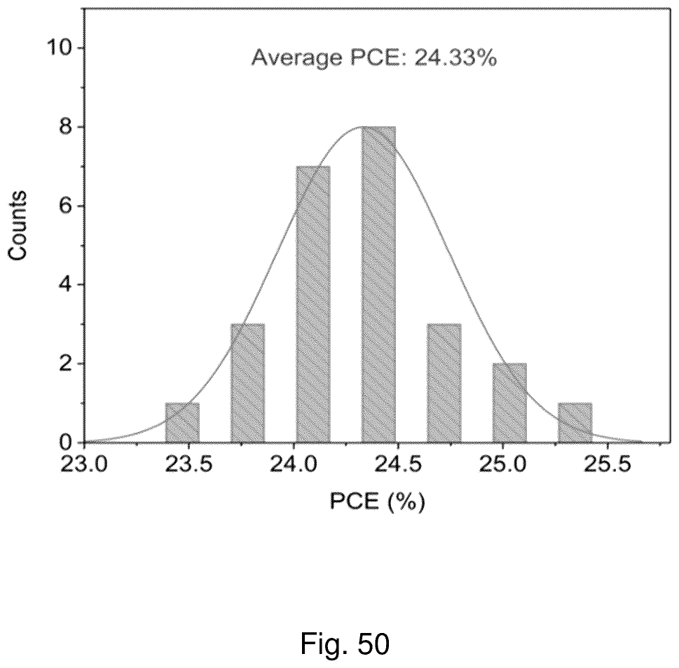

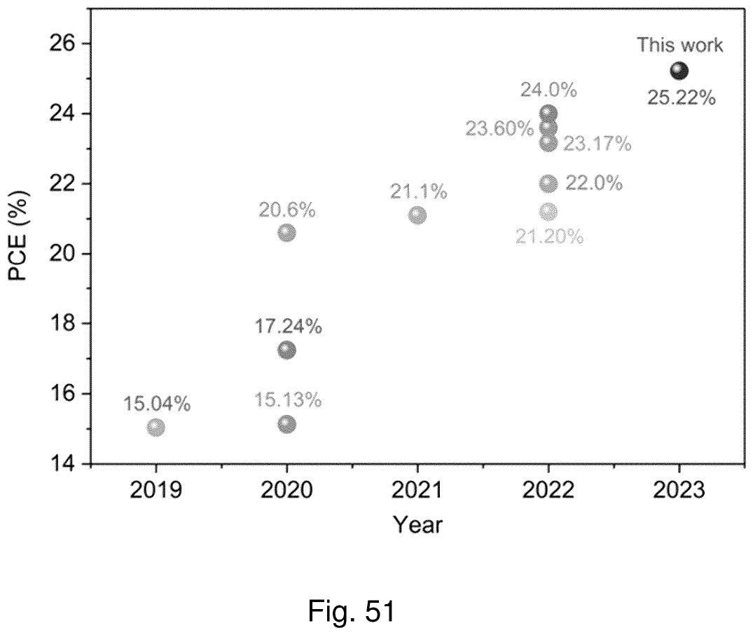

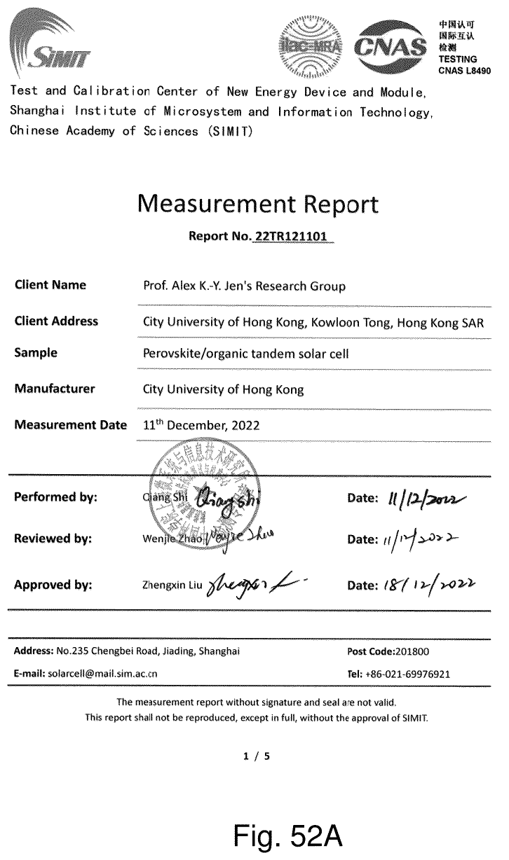

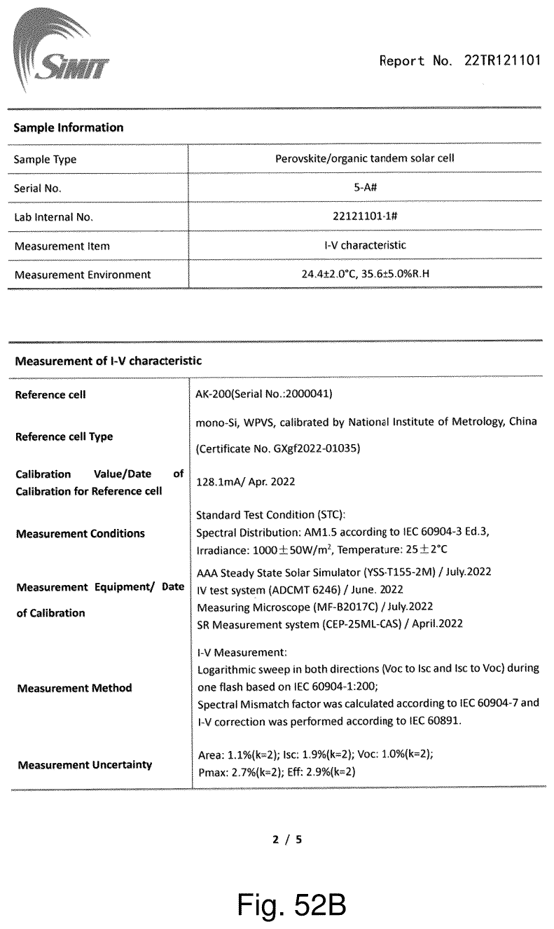

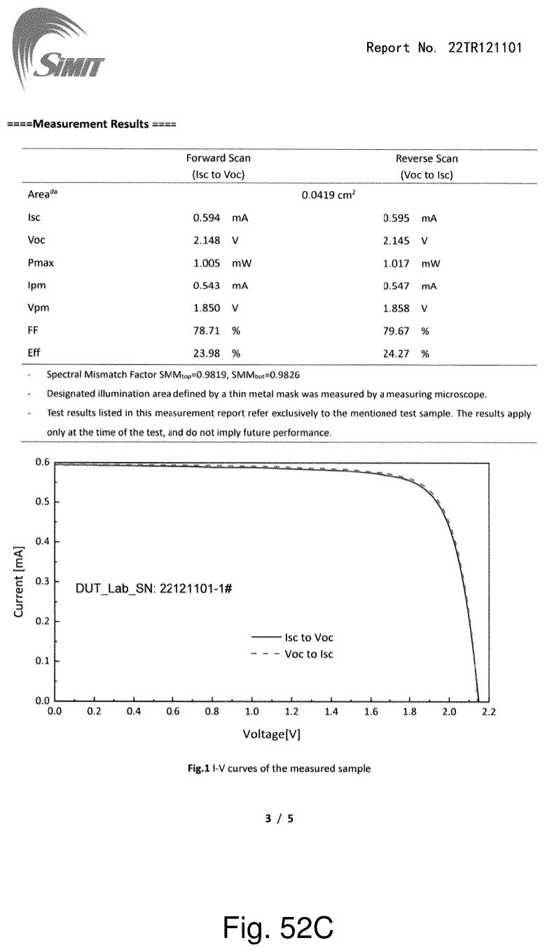

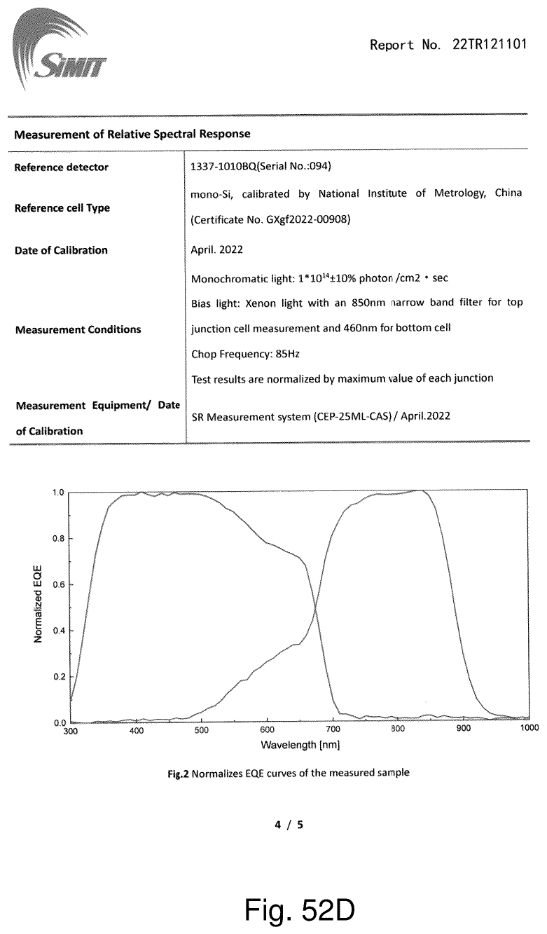

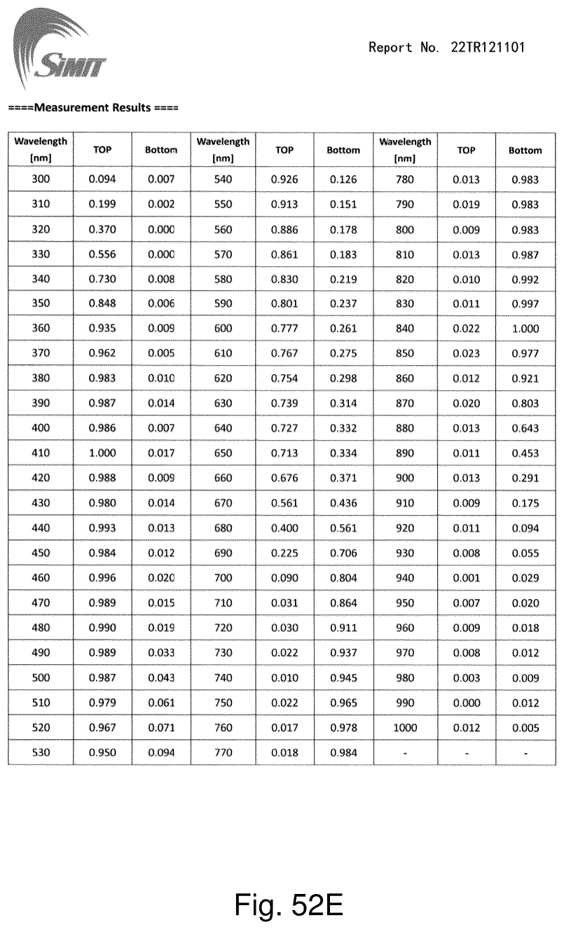

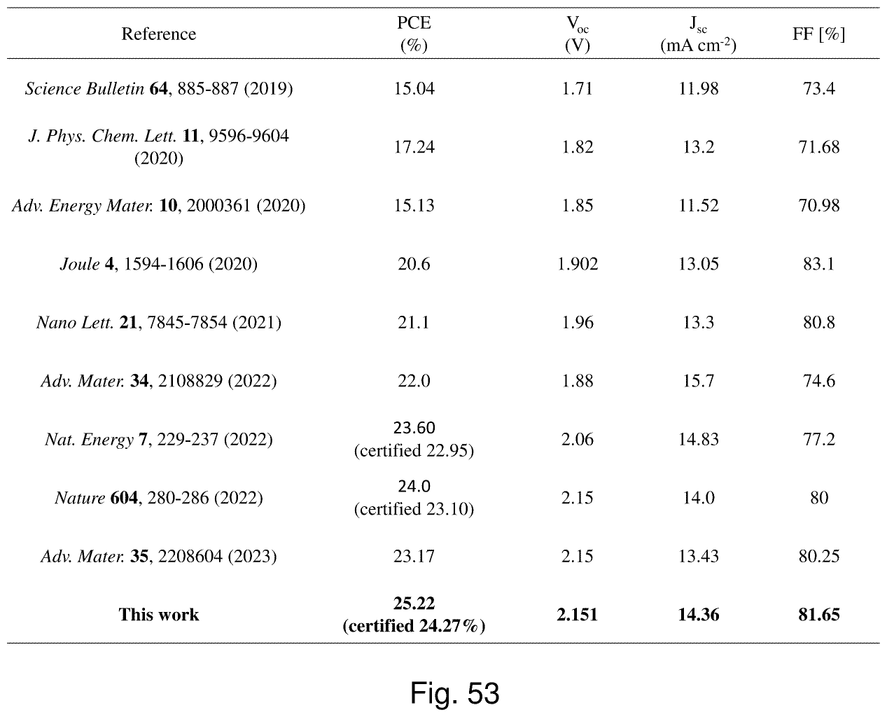

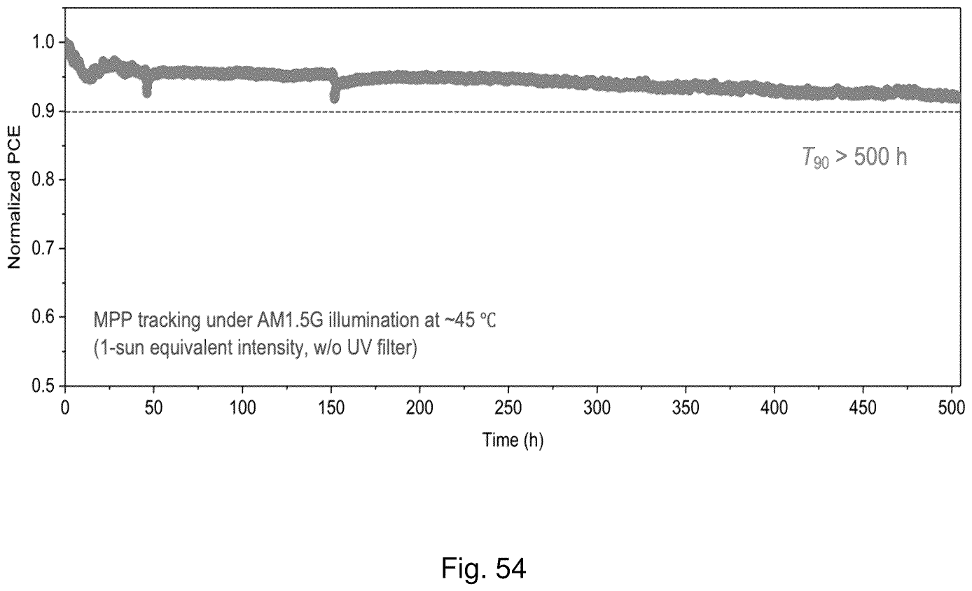

The average PCE of 24.33% was calculated from 25 individual tandem cells that were fabricated from different batches, as shown in FIG. 50. An encapsulated tandem cell (with a fixed mask) was also sent to an independent photovoltaic calibration institute (SIMIT) for certification, realizing a certified efficiency of 24.27%. It is believed that this value represents the highest certified PCE among the reported PO-TSCs (FIGS. 51, 52A-52E, and 53). An encapsulated tandem cell was subjected to 1-sun illumination (AM 1.5G spectrum, without a UV filter) in an N₂-filled chamber, which retained 92% of its initial PCE after 500 hours of continuous operation at ˜45° C. (FIG. 54).

The invention has been given by way of example only, and various other modifications of and/or alterations to the described embodiment may be made by persons skilled in the art without departing from the scope of the invention as specified in the appended claims.

1. A perovskite layer for use in a solar cell comprising a mixture of a halide perovskite and a sulfonyl naphthoquinone-based compound having a structure of Formula (I): 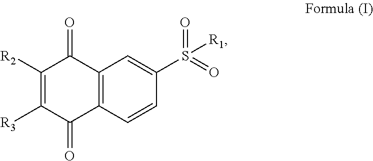
wherein:
R₁ is independently selected from one of OM, OR₄, and NR₅R₆, with M being a cation, R₄, R₅ and R₆ being independently selected from a hydrogen and a substituent; and
R₂ and R₃ are independently selected from a hydrogen and a substituent, or R₂ and R₃ may form a fused ring.

2. The perovskite layer as claimed in claim 1, wherein:
M is selected from the group consisting of H⁺, Na⁺, K⁺, Cs⁺, Rb⁺, Sr²⁺, Ca²⁺, Ni²⁺, Cu²⁺, Co²⁺, Zn²⁺, Mg²⁺, Ba²⁺, Li⁺, ammonium ion, and aminum ion;
R₂, R₃, R₄, R₅ and R₆ are independently selected from the group consisting of hydrogen, substituted or unsubstituted C1 to C4 alkyl, substituted or unsubstituted C2 to C6 alkenyl, substituted or unsubstituted C2 to C6 alkynyl, halogen, substituted or unsubstituted C6 to C10 aryl, —CN, —C(═O)OH, —C(═O)H, —C(═O)R₇, —OR₇, —SH, —SR₇, —NH₂, —NHR₆, —N(R₇)₂, —Si(R₇)₃, —OSi(R₇)₃, —S(O)OH and —P(O)(OH)₂ where R₇ is an alkyl or a phenyl; and
when R₂ and R₃ form a fused ring, it comprises a substituted or unsubstituted 6-membered to 14-membered ring.

3. The perovskite layer as claimed in claim 2, wherein the sulfonyl naphthoquinone-based compound is any one of Formula (II), (III), (IV), (V), (VI), or (VII): 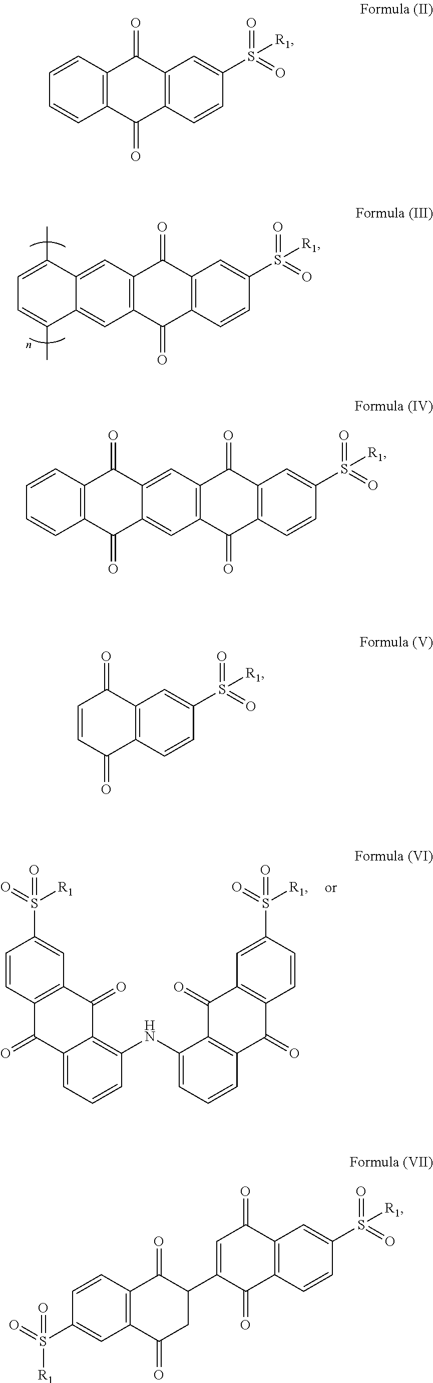
with R₁ as defined above; and n being 1-1000.

4. The perovskite layer as claimed in claim 3, wherein the sulfonyl naphthoquinone-based compound of Formula (II) is selected from any one of Formula (VIII), Formula (IX), or Formula (X): 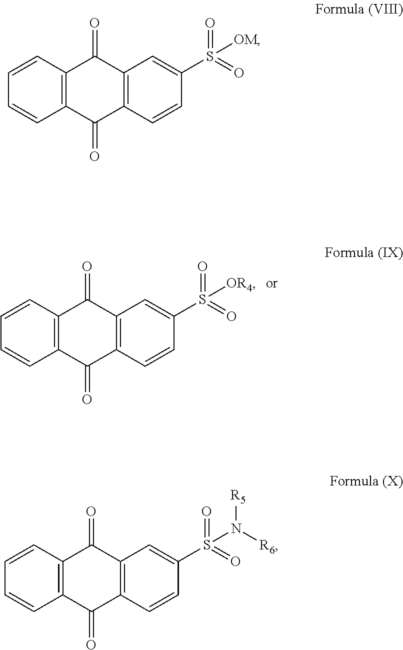
with M being H⁺, ammonium ion or aminum ion; R₄ being C1 to C4 alkyl; R₅ and R₆ being independently selected from hydrogen and C1 to C4 alkyl.

5. The perovskite layer as claimed in claim 4, wherein the sulfonyl naphthoquinone-based compound of Formula (VIII) is selected from any one of Formula (VIIIa), (VIIIb), and (VIIIc):

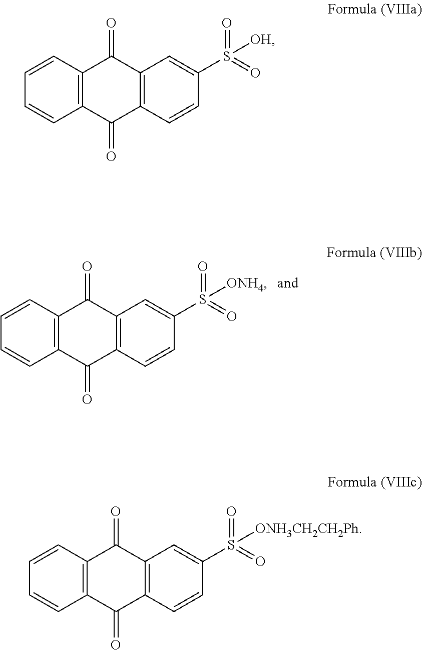

6. The perovskite layer as claimed in claim 4, wherein the sulfonyl naphthoquinone-based compound comprises a structure of Formula (IXa):

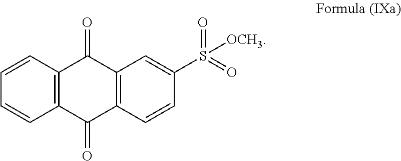 [SMILES: COS(=O)(=O)c1ccc2c(c1)C(=O)c1ccccc1C2=O]

7. The perovskite layer as claimed in claim 4, wherein the sulfonyl naphthoquinone-based compound comprises a structure of Formula (Xa):

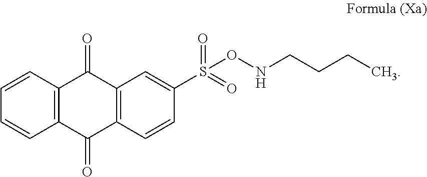 [SMILES: CCCCNOS(=O)(=O)c1ccc2c(c1)C(=O)c1ccccc1C2=O]

8. The perovskite layer as claimed in claim 1, wherein the halide perovskite comprises a crystal grain structure of [A¹B⁺²X⁻¹₃], with A⁺¹ being an A-site monovalent cation, B⁺² being a B-site divalent cation, and X⁻¹ being a halide anion.

9. The perovskite layer as claimed in claim 8, wherein the A-site monovalent cation is selected from the group consisting of formamidinium (FA⁺), methylammonium (MA⁺), ethylammonium (EA⁺), guanidinium (GA⁺), Cs⁺, Rb⁺ and a combination thereof; the B-site divalent cation is selected from the group consisting of Pb²⁺, Sn²⁺, Ge²⁺ and a combination thereof; and the halide anion is selected from the group consisting of I⁻, Br⁻, Cl⁻ and a combination thereof.

10. The perovskite layer as claimed in claim 9, wherein the halide perovskite is selected from any one of CsPb_{0.5}Sn_{0.5}I₃, FAPbI₃, MA_{0.25}FA_{0.75}PbI_{2.2}Br_{0.6}Cl_{0.2}, Cs_{0.02}FA_{0.96}MA_{0.02}PbI_{0.99}Cl_{0.01}, MAPb_{0.92}Sn_{0.05}I₃, (FA_{0.95}MA_{0.05})_{0.95}Cs_{0.05}Pb(I_{0.96}Br_{0.04})₃, Rb_{0.1}FA_{0.8}GA_{0.1}Pb_{0.6}Ge_{0.4}I₃, and (FA_{0.92}MA_{0.08})_{0.9}Cs_{0.1}Pb(I_{0.92}Br_{0.08})₃, and Cs_{0.2}FA_{0.8}Pb(I_{0.6}Br_{0.4})₃.

11. The perovskite layer as claimed in claim 8, wherein the crystal grain structure of [A⁺¹B⁺²X⁻¹₃] includes grain boundaries at which the sulfonyl naphthoquinone-based compound accommodates.

12. The perovskite layer as claimed in claim 1 containing about 0.3 mol % to about 1 mol % of the sulfonyl naphthoquinone-based compound.

13. The perovskite layer as claimed in claim 1 further comprising about 3 mol % of MAPbCl₃ and 5 mol % of 4-guanidinobenzoic acid.

14. The perovskite layer as claimed in claim 1 having a thickness of about 260 nm.

15. A method for fabricating the perovskite layer as claimed in claim 1, comprising the steps of:
a) providing a solution mixture including a halide perovskite precursor and a sulfonyl naphthoquinone-based compound selected from any one of Formula (VIII), Formula (IX), or Formula (X): 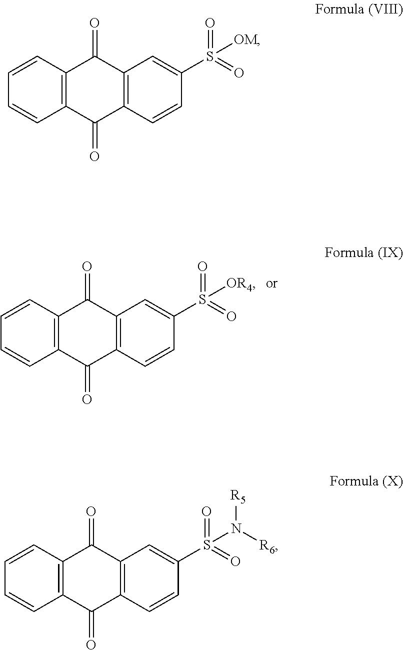
with M being H⁺, ammonium ion or aminum ion; R₄ being C1 to C4 alkyl; R₅ and R₆ being independently selected from hydrogen and C1 to C4 alkyl;
b) spin-coating the solution mixture on a substrate; and
c) annealing the spin-coated solution mixture to form the perovskite layer.

16. The method as claimed in claim 15, wherein step a) comprises the steps of:
a1) providing a first reaction mixture including halides of formamidinium, methylammonium, and cesium; and
a2) mixing the first reaction mixture with about 0.3 mol % to about 1 mol % of the sulfonyl naphthoquinone-based compound to form a second reaction mixture.

17. The method as claimed in claim 16, wherein the halides comprises CsI, CsBr, FAI, FABr, PbI₂, and PbBr₂.

18. The method as claimed in claim 16, wherein the first reaction mixture further comprises about 3 mol % of MAPbCl₃ and about 5 mol % of 4-guanidinobenzoic acid.

19. The method as claimed in claim 15, wherein step a) further comprises step a3) converting sodium anthraquinone-2-sulfonate to the sulfonyl naphthoquinone-based compound.

20. The method as claimed in claim 15, wherein step b) comprises step b1) adding an anti-solvent agent of chlorobenzene onto the center of the spin-coated solution mixture about 10 seconds before completing the spin-coating process.

21. The method as claimed in 15, wherein the spin-coating is performed at about 4000 rpm to about 6500 rpm with a ramping rate of about 1500 rpm si.

22. The method as claimed in claim 15, after step c), further comprising the steps of:
spin-coating a surface passivating agent of piperazinium iodide (PI) on the perovskite layer; and
annealing the PI-coated perovskite layer.

23. A solar cell comprising:
a first hole transport layer;
a first electron transport layer; and
a first active layer of the perovskite layer as claimed in claim 1 that is disposed between the first hole transport layer and the first electron transport layer.

24. The solar cell as claimed in claim 23, wherein the first active layer is in direct contact with the first hole transport layer and the first electron transport layer.

25. The solar cell as claimed in claim 24, wherein the first hole transport layer is disposed on a transparent conductive layer that is disposed on a transparent substrate and the first electron transport layer is disposed on a first blocking layer that is disposed on a first metal layer.

26. The solar cell as claimed in claim 25, wherein the first hole transport layer is in direct contact with a transparent conductive layer and the first electron transport layer is in direct contact with the first blocking layer.

27. The solar cell as claimed in claim 25, wherein the transparent substrate is selected from the group consisting of glass, polymethyl methacrylate (PMMA), polycarbonate (PC), general-purpose polystyrene (GPPS), polyethylene glycol terephthalate (PET), polyethylene naphthalate (PEN), polydimethylsiloxane (PDMS), styrene-ethylene-butylene-styrene (SEBS), ethylene terephthalateco-1,4-cylclohexylenedimethylene terephthalate (PETG), acrylonitrile butadiene styrene copolymers (ABS), polypropylene (PP), polyamide (PA), acrylonitrile-styrene copolymer (AS), and a combination thereof.

28. The solar cell as claimed in 25, wherein the transparent conductive layer is selected from the group consisting of Indium Tin Oxide (ITO), Aluminum Zinc Oxide (AZO), Fluorine Tin Oxide (FTO), graphene, poly(3,4-ethylenedioxythiophene):poly(styrene sulfonate) (PEDOT:PSS), Ag nanowire, Cu nanowire and a combination thereof.

29. The solar cell as claimed in claim 25, wherein first hole transport layer is selected from the group consisting of poly(triaryl amine) (PTAA), PEDOT:PSS, NiOx, 2,2′,7,7′-Tetrakis[N,N-di(4-methoxyphenyl)amino]-9,9′-spirobifluorene (Spiro-OMeTAD), DC-PA, MoOₓ and a combination thereof.

30. The solar cell as claimed in claim 25, wherein the first electron transport layer is selected from the group consisting of PC₆₁BM, C₆₀, SnO₂, PNDIT-F3N and a combination thereof.

31. The solar cell as claimed in claim 25, wherein the first blocking layer is selected from the group consisting of bathocuproine (BCP), bis-C₆₀, SnOₓ, Zr(acac)₂, MoOₓ and a combination thereof.

32. The solar cell as claimed in claim 25, wherein the first metal layer is selected from the group consisting of Ag, Cu, Au, Al, W, Fe, Pt and a combination thereof.

33. The solar cell as claimed in claim 23 comprises a band gap of about 1.81 eV.

34. The solar cell as claimed in claim 23 comprises a power conversion efficiency of about 18.98% to about 19.58%.

35. The solar cell as claimed in claim 23 comprises a power conversion efficiency of 95% of its initial value upon AM1.5G illumination for 500 hours at about 45° C.

36. The solar cell as claimed in claim 25, further comprising a subcell disposed on and in direct contact with the first metal layer.

37. The solar cell as claimed in claim 36, further including an anti-reflection layer to which the transparent conductive layer and the transparent substrate are disposed on and in direct contact therewith.

38. The solar cell as claimed in claim 36, wherein the anti-reflection layer is selected from the group consisting of MgF₂, LiF, PDMS and a combination thereof.

39. The solar cell as claimed in claim 36, wherein the subcell comprises:
a second hole transport layer;
a second electron transport layer; and
a second active layer that is disposed between the second hole transport layer and the second electron transport layer.

40. The solar cell as claimed in claim 39, wherein the second active layer is in direct contact with the second hole transport layer and the second electron transport layer.

41. The solar cell as claimed in claim 39, wherein the second active layer is selected from the group consisting of a perovskite photovoltaic material, a Si photovoltaic material, a CIGS photovoltaic material, a CdTe photovoltaic material, an organic photovoltaic material and a combination thereof.

42. The solar cell as claimed in claim 40, wherein the second hole transport layer is disposed on the first metal layer and the second electron transport layer is disposed on a second metal layer.

43. The solar cell as claimed in claim 39, wherein the second hole transport layer is in direct contact with the first metal layer and the second electron transport layer is in direct contact with the second metal layer.

44. The solar cell as claimed in claim 40, wherein the second hole transport layer is disposed on the first metal layer and the second electron transport layer is disposed on the second metal layer.

45. The solar cell as claimed in claim 39, wherein the second hole transport layer is in direct contact with the first metal layer and the second electron transport layer is in direct contact with the second blocking layer.

46. The solar cell as claimed in claim 42 comprises a perovskite-organic tandem solar cell.

47. The solar cell as claimed in claim 46, wherein the perovskite-organic tandem solar cell comprises the first active layer of Cs_{0.2}FA_{0.8}Pb(I_{0.6}Br_{0.4})₃ and the second active layer of PM6:Y6:P₇₁BM.

48. The solar cell as claimed in claim 46, wherein the perovskite-organic tandem solar cell has a power conversion efficiency of about 24.27% to about 25.22%.

49. The solar cell as claimed in claim 46, wherein the perovskite-organic tandem solar cell has a power conversion efficiency of 92% of its initial value upon AM1.5G illumination for 500 hours at about 45° C.
